(19) United States
(12) Patent Application Publication
(10) Pub. No.: US 2025/0386788 A1
(43) Pub. Date: Dec. 25, 2025

(54) USE OF PAIRED GENES IN HYBRID BREEDING

(71) Applicant: KWS SAAT SE & Co. KGaA, Einbeck (DE)

(72) Inventors: Katrin DIETRICH, Einbeck (DE); Klaus SCHMIDT, Einbeck (DE)

(73) Assignee: KWS SAAT SE & Co. KGaA, Einbeck (DE)

(21) Appl. No.: 19/106,507

(22) PCT Filed: Aug. 25, 2023
(86) PCT No.: PCT/EP2023/073320

§ 371 Date: Feb. 25, 2025

(30) Foreign Application Priority Data
Aug. 26, 2022 (EP) 22192484.8

(51) Int. Cl. A01H 1/04; A01H 1/02; C12Q 1/6895

(52) U.S. Cl. CPC A01H 1/045; A01H 1/023; C12Q 1/6895; C12Q 2600/13

(57) ABSTRACT

The present invention relates to linked gene pairs for use in plant breeding, such as hybrid breeding. The present invention in particular relates to the Myb80 and Dwarf11 genes, mutations of which respectively result in genetic male sterility and dwarfism. Combined mutations allow selection of genetic male sterile plants based on a dwarfism phenotype.

### FIELD OF THE INVENTION

The invention relates to hybrid seed production, in particular in cereals, involving the use of male sterile plants. The invention further relates to methods for identifying such male sterile plants.

### BACKGROUND OF THE INVENTION

For every hybrid seed production system, a prerequisite is the availability of male sterile lines. There are two different types of male sterility described: Cytoplasmic male sterility (CMS) caused by a mutation in the mitochondrial genome as well as Genic male sterility (GMS) caused by mutations in the nuclear genome. CMS and GMS facilitate hybrid seed production for many crops and thus allow breeders to harness yield gains associated with hybrid vigor (heterosis).

Cytoplasmic male sterility (CMS) is a maternally inherited condition in which a plant is unable to produce functional pollen. In CMS, layers of interaction between mitochondrial and nuclear genes control its male specificity, occurrence, and restoration of fertility. It occurs in many plant species and is often associated with chimeric mitochondrial open reading frames. In a number of cases, transcripts originating from these altered open reading frames are translated into unique proteins that appear to interfere with mitochondrial function and pollen development. Nuclear restorer (Rf or Fr) genes function to suppress the deleterious effects of CMS-associated mitochondrial abnormalities by diverse mechanisms. Due to the different origins of the causative defect, the CMS is only inherited through the female germline, and to regain full fertility requires an additional factor in the hybrid seeds (Restorer gene) This makes breeding complicated, and sometimes it is problematic to regain back full fertility due to the lack of functional restorer genes.

Genic male sterility (GMS) is caused only by genes encoded in the nuclear genome. In GMS, nuclear Male sterility (Ms) genes control the male sterility condition without influence of cytoplasmic sequences. In the simplest genetic model, there are three possible genotypes for the nuclear locus Ms, in which the male sterile phenotype is conditioned by recessive ms alleles. A Mendelian inheritance pattern can be observed, in which the offspring of a male sterile genotype (female line) could be entirely male fertile or segregate 50% male sterile: 50% male fertile depending on whether the parental line (male fertile) is homozygous or heterozygous, respectively. The use of GMS in plant breeding and hybrid seed production involves three different lines: i) a male sterile (female parent), ii) a maintainer, and iii) a restorer (male parent) line. The male sterile line is maintained using pollen of a maintainer line, which presents identical genotype (isoline), except for the presence of a dominant Ms allele.

When using GMS systems, fertility may already be restored in the heterozygous stage, as the sterility is only observed in plants homozygous for the sterility allele of the gene. This causes other problems, as there is no easy way to produce the male sterile female plants, and importantly also to identify the male sterile female plants, in particular at an early stage, such as at the seed stage.

It is an objective of the present invention to address one or more of the above shortcomings.

### SUMMARY OF THE INVENTION

The invention describes the discovery of a very specific pair of genes in the genome of cereals providing a specific combination of expected phenotypes: male sterility and reduced plant height/smaller seed size.

This combination of phenotypes can be used for the development of an alternative hybrid system in different cereal species. For some cereal species, there are no hybrid systems available at all (e.g. oat), for other cereal species, this alternative hybrid system is supposed to be beneficial compared to the existing hybrid systems.

Getting rid of the current limitations of the existing hybrid systems in cereals and develop a new system applicable in all cereals also opens new possibilities for niche crops like oat, where the breeding progress is slow. As it was observed in other crops, a functional hybrid system is expected to result in a significant increase in yield due to the heterosis.

The present invention therefore is to provide a system to be able to maintain and produce male sterile plants form a GMS system in a cost-efficient way via coupling the male sterility phenotype with an easy to screen phenotypic marker. As the sterility can only be seen at the late plant developmental phase, the phenotype linked to the sterility should be visible already at the seed level. However, there might be other suitable phenotypes which can be screened for already at very early phases of plant development.

The general principle of finding specific gene pairs in the genome of a plant species may equally be applied to get a combination of male sterility and a morphological trait in all plant species including dicot species. So, the system of the present invention can be applied to crop species where no hybrid system is even foreseeable at the moment.

One aspect of the present invention therefore provides a general method of finding specific gene pairs in the genome of a plant species to get a combination of male sterility and a morphological trait in all plant species including dicot species.

A specific gene pair can be selected in a manner that one gene causes male sterility (for instance Myb80), and the other gene relates to a particular phenotype (for instance Dwarf11). The screening parameters are set such that the physical distance between the two genes is less than 1 Mbp. The assumption is that this physical distance is sufficient for a close genetic linkage of the two genes, so they are supposed to be inherited as one genetic locus.

Another aspect of the present invention is to provide a system to be able to maintain and produce male sterile plants form a GMS system in a cost-efficient way via coupling the male sterility phenotype with an easy to screen phenotypic marker.

A more specific aspect of the present invention is to provide a system, which is capable to maintain and produce male sterile plants from a GMS system including Myb80 and Dwarf11 mutants.

The present invention is in particular captured by any one or any combination of one or more of the below numbered statements 1 to 106, with any other statement and/or embodiments.

1. A method for generating or modifying a plant or plant part, comprising
a) providing a plant or plant part not expressing or having reduced expression, activity, and/or stability of the MYB80 gene product; and/or having a (homozygous or heterozygous) mutation in the Myb80 gene or regulatory sequence thereof;
b) reducing or eliminating expression, activity, and/or stability of the DWARF11 gene product in said plant or plant part; and/or introducing a (homozygous or heterozygous) mutation in the Dwarf11 gene or regulatory sequence thereof.

2. The method according to statement 1, wherein said plant or plant part comprises one or more (homozygous or heterozygous) mutation in the MYB80 gene.

3. A method for generating or modifying a plant or plant part, comprising
a) providing a plant or plant part not expressing or having reduced expression, activity, and/or stability of the DWARF11 gene product; and/or having a (homozygous or heterozygous) mutation in the Dwarf11 gene or regulatory sequence thereof;
b) reducing or eliminating expression, activity, and/or stability of the MYB80 gene product in said plant or plant part; and/or introducing a (homozygous or heterozygous) mutation in the Myb80 gene or regulatory sequence thereof.

4. The method according to statement 3, wherein said plant or plant part comprises one or more (homozygous or heterozygous) mutation in the DWARF11 gene.

5. A method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) (homozygously or heterozygously) mutating the Myb80 gene and the Dwarf11 gene or regulatory sequences thereof; or a combination of b1) and b2).

6. The method according to any of statements 1 to 5, wherein reducing or eliminating expression, activity, and/or stability comprises introducing a (homozygous or heterozygous) mutation in the gene.

7. The method according to any of statements 1 to 6, wherein reducing or eliminating expression, activity, and/or stability comprises knocking down the gene transcript or knocking out the gene, preferably by RNAi or CRISPR/Cas.

8. A method for generating a plant or plant part, comprising
a) crossing a first plant and a second plant; wherein said first plant and/or said second plant have a heterozygous or homozygous mutation in the MYB80 gene and the DWARF11 gene on the same chromosome;
b) harvesting seeds.

9. The method according to statement 8, further comprising
c) selecting seeds or plants grown therefrom comprising a homozygous mutation in the DWARF11 gene.

10. The method according to any of statements 1 to 9 wherein said mutation is a nonsense or missense mutation.

11. The method according to any of statements 1 to 10, wherein said mutation is a frameshift mutation.

12. The method according to any of statements 1 to 11, wherein said mutation is an indel mutation.

13. The method according to any of statements 1 to 12, wherein said mutation is a dominant (negative) mutation or a recessive mutation.

14. The method according to any of statements 1 to 13, wherein said mutation is a knock-out or knock-down mutation.

15. The method according to any of statements 1-14, wherein said mutation is in the first exon.

16. The method according to any of statements 1 to 15, wherein said mutation is in the coding sequence, a splicing signal or affecting a splice signal, or a regulatory element.

17. The method according to any of statements 1 to 16, wherein said mutation is introduced by (random) mutagenesis, preferably TILLING.

18. The method according to any of statements 1 to 17, wherein said mutation is introduced by site-directed mutagenesis.

19. The method according to any of statements 1 to 18, wherein said mutation is introduced by gene-editing, preferably by CRISPR/Cas.

20. The method according to any of statements 1 to 19, wherein said method is a method for generating or modifying a plant or plant part which is male sterile.

21. The method according to any of statements 1 to 20, wherein said method is a method for generating or modifying a plant or plant part which is genetic male sterile.

22. The method according to any of statements 1 to 21, wherein said plant or plant part is from the family of Poaceae.

23. The method according to any of statements 1 to 22, wherein said plant or plant part is from the subfamily of Pooideae, Panicoideae, Chloridoideae, Pharoideae, Bambusoideae, or Oryzoideae.

24. The method according to any of statements 1 to 23, wherein said plant or plant part is from the genus Aegilops, Brachypodium, Cenchrus, Chasmanthium, Digitaria, Eleusine, Eragrostis, Hordeum, Leersia, Lolium, Miscanthus, Oropetium, Oryza, Panicum, Paspalum, Pharus, Phyllostachys, Saccharum, Secale, Setaria, Sorghum, Thinopyrum, Triticum, Urochloa, Avena, Poa, Phleum, Festuca, or Deschampsia.

25. The method according to any of statements 1 to 24, wherein said plant or plant part is selected from the species Aegilops tauschii, Brachypodium distachyon, Brachypodium mexicanum, Brachypodium stacei, Brachypodium sylvaticum, Cenchrus purpureus, Chasmanthium laxum, Digitaria exilis, Eleusine coracana, Eleusine coracana, Eragrostis curvula, Eragrostis tef, Hordeum vulgare, Leersia perrieri, Lolium perenne, Miscanthus sinensis, Oropetium thomaeum, Oryza alta, Oryza brachyantha, Oryza glaberrima, Oryza glumipatula, Oryza longistaminata, Oryza meridionalis, Oryza nivara, Oryza punctata, Oryza rufipogon, Oryza sativa Indica Group, Oryza sativa Japonica Group, Panicum hallii FIL2, Panicum hallii HAL2, Panicum virgatum, Paspalum vaginatum, Pharus latifolius, Phyllostachys edulis, Saccharum spontaneum, Secale cereale, Setaria italica, Setaria viridis, Sorghum bicolor, Thinopyrum intermedium, Triticum aestivum, Triticum dicoccoides, Triticum spelta, Triticum turgidum, or 25 Urochloa fusca.

26. The method according to any of statements 1 to 25, wherein said plant or plant part is a crop plant or plant part.

27. The method according to any of statements 1 to 26, wherein said MYB80 gene and said DWARF11 gene are located on the same chromosome.

28. The method according to any of statements 1 to 27, wherein said MYB80 gene and said DWARF11 gene are located on the same chromosomal arm.

29. The method according to any of statements 1 to 28 wherein the physical distance between said MYB80 gene and said DWARF11 gene in the genome of said plant or plant part is at most 1 Mbp.

30. The method according to any of statements 1 to 29, wherein the genetic distance between said MYB80 gene and said DWARF11 gene in the genome of said plant or plant part is at most 1 CM.

31. The method according to any of statements 1 to 23, wherein said plant or plant part is not from the genus Zea.

32. The method according to any of statements 1 to 23, wherein said plant or plant part is not from the species Zea mays.

33. The method according to any of statements 1 to 23, wherein said plant or plant part is not from the species Triticum urartu.

34. A method for selecting a plant or plant part, comprising
a) providing a mixture of plants or plant parts resulting from a cross between a first plant and a second plant; wherein said first plant and/or said second plant have a heterozygous or homozygous mutation in the MYB80 gene and the DWARF11 gene on the same chromosome;
b) selecting a plant or plant part comprising a homozygous mutation in the DWARF11 gene.

35. The method according to any of statements 9 to 34, wherein selecting a plant or plant part comprising a homozygous mutation in the DWARF11 gene comprises phenotypic selection.

36. A method for identifying and/or selecting a plant or plant part, comprising screening for the presence of a homozygous mutation in the DWARF11 gene in (the genome of) a plant or plant part resulting from a cross between a first plant and a second plant; wherein said first plant and/or said second plant have a heterozygous mutation in the MYB80 gene and the DWARF11 gene on the same chromosome; and optionally selecting a plant or plant part comprising a homozygous mutation in the DWARF11 gene.

37. The method according to statement 36, wherein screening for the presence of a homozygous mutation in the DWARF 11 gene comprises phenotypic screening.

38. The method according to statement 37, wherein said phenotypic selection or screening comprises selection or screening based on plant grain size, grain shape, or grain weight.

39. The method according to statement 37 or 38, wherein said phenotypic selection or screening comprises selection or screening based on plant height.

40. The method according to any of statements 8 to 39, wherein said first plant and said second plant are the same.

41. The method according to any of statements 8 to 39, wherein said first plant and said second plant are different.

42. The method according to any of statements 34 to 41, wherein said method is a method for selecting and/or identifying a plant or plant part which is male sterile.

43. The method according to any of statements 34 to 42, wherein said method is a method for selecting and/or identifying a plant or plant part which is genetic male sterile.

44. The method according to any of statements 34 to 43, wherein said method is a method for selecting and/or identifying a dwarf plant or plant part which is male sterile.

45. The method according to any of statements 34 to 44, wherein said method is a method for selecting and/or identifying a dwarf plant or plant part which is genetic male sterile.

46. The method according to any of statements 1 to 45, wherein expression, activity, and/or stability of MYB80 and/or DWARF11 is reduced compared to a predetermined threshold.

47. The method according to any of statements 1 to 46, wherein expression, activity, and/or stability of MYB80 and/or DWARF 11 is at least 50%, preferably at least 80%, more preferably at least 90% reduced compared to a predetermined threshold.

48. The method according to statement 47, wherein said threshold is the expression level, activity, and/or stability in a plant or plant part lacking a mutation in the MYB80 gene and/or DWARF11 gene.

49. The method according to any of statements 1 to 48, wherein said plant part is not propagation material.

50. The method according to any of statements 1 to 48, wherein said plant part is a plant organ, tissue, or cell.

51. The method according to statement 50, wherein said plant part is seed, pollen, oocyte, protoplast, inflorescence, embryo, or callus.

52. The method according to any of statements 1 to 51, wherein said plant or plant part is mutagenized.

53. The method according to any of statements 1 to 52, wherein said plant or plant part is transgenic or genome-edited.

54. A plant or plant part generated, modified, identified, or selected according to the method of any of statements 1 to 53, or offspring thereof.

55. A plant or plant part not expressing or having reduced expression, activity, and/or stability of MYB80 and DWARF11.

56. The plant or plant part according to statement 55, comprising a mutation in the MYB80 gene and the DWARF11 gene (on the same chromosome).

57. The plant or plant part according to statement 56, wherein (both) said mutation(s) is (are) heterozygous.

58. The plant or plant part according to statement 56, wherein (both) said mutation(s) is (are) homozygous.

59. The plant or plant part according to any of statements 56 to 58 wherein said mutation is a nonsense or missense mutation.

60. The plant or plant part according to any of statements 56 to 59, wherein said mutation is a frameshift mutation.

61. The plant or plant part according to any of statements 56 to 60, wherein said mutation is an indel mutation.

62. The plant or plant part according to any of statements 56 to 61, wherein said mutation is a dominant (negative) mutation or a recessive mutation.

63. The plant or plant part according to any of statements 56 to 62, wherein said mutation is a knock-out or knock-down mutation.

64. The plant or plant part according to any of statements 56 to 63, wherein said mutation is in the coding sequence, a splicing signal, or a regulatory element.

65. The plant or plant part according to any of statements 56 to 64, wherein said mutation is in the first exon.

66. The plant or plant part according to any of statements 56 to 64, wherein said mutation is introduced by (random) mutagenesis, preferably TILLING.

67. The plant or plant part according to any of statements 56 to 65, wherein said mutation is introduced by site-directed mutagenesis.

68. The plant or plant part according to any of statements 56 to 67, wherein said mutation is introduced by gene-editing, preferably by CRISPR/Cas.

69. The plant or plant part according to any of statements 55 to 68, wherein said plant or plant part which is male sterile.

70. The plant or plant part according to any of statements 55 to 69, wherein said plant part is genetic male sterile.

71. The plant or plant part according to any of statements 55 to 70, wherein said plant or plant part is from the family of Poaceae.

72. The plant or plant part according to any of statements 55 to 71, wherein said plant or plant part is from the subfamily of Pooideae, Panicoideae, Chloridoideae, Pharoideae, Bambusoideae, or Oryzoideae.

73. The plant or plant part according to any of statements 55 to 72, wherein said plant or plant part is from the genus Aegilops, Brachypodium, Cenchrus, Chasmanthium, Digitaria, Eleusine, Eragrostis, Hordeum, Leersia, Lolium, Miscanthus, Oropetium, Oryza, Panicum, Paspalum, Pharus, Phyllostachys, Saccharum, Secale, Setaria, Sorghum, Thinopyrum, Triticum, Urochloa, Avena, Poa, Phleum, Festuca, or Deschampsia.

74. The plant or plant part according to any of statements 55 to 73, wherein said plant or plant part is selected from the species Aegilops tauschii, Brachypodium distachyon, Brachypodium mexicanum, Brachypodium stacei, Brachypodium sylvaticum, Cenchrus purpureus, Chasmanthium laxum, Digitaria exilis, Eleusine coracana, Eleusine coracana, Eragrostis curvula, Eragrostis tef, Hordeum vulgare, Leersia perrieri, Lolium perenne, Miscanthus sinensis, Oropetium thomaeum, Oryza alta, Oryza brachyantha, Oryza glaberrima, Oryza glumipatula, Oryza longistaminata, Oryza meridionalis, Oryza nivara, Oryza punctata, Oryza rufipogon, Oryza sativa Indica Group, Oryza sativa Japonica Group, Panicum hallii FIL2, Panicum hallii HAL2, Panicum virgatum, Paspalum vaginatum, Pharus latifolius, Phyllostachys edulis, Saccharum spontaneum, Secale cereale, Setaria italica, Setaria viridis, Sorghum bicolor, Thinopyrum intermedium, Triticum aestivum, Triticum dicoccoides, Triticum spelta, Triticum turgidum, or Urochloa fusca.

75. The plant of plant part according to any of statements 55 to 74, wherein said plant or plant part is a crop plant or plant part.

76. The plant or plant part according to any of statements 55 to 75, wherein said plant or plant part is a turf grass.

77. The plant or plant part according to any of statements 55 to 76, wherein said MYB80 gene and said DWARF11 gene are located on the same chromosome.

78. The plant or plant part according to any of statements 55 to 77 wherein said MYB80 gene and said DWARF11 gene are located on the same chromosomal arm.

79. The plant or plant part according to any of statements 55 to 78 wherein the physical distance between said MYB80 gene and said DWARF11 gene in the genome of said plant or plant part is at most 1 Mb.

80. The plant or plant part according to any of statements 55 to 79, wherein the genetic distance between said MYB80 gene and said DWARF11 gene in the genome of said plant or plant part is at most 1 cM.

81. The plant or plant part according to any of statements 55 to 72, wherein said plant or plant part is not from the genus Zea.

82. The plant or plant part according to any of statements 55 to 72, wherein said plant or plant part is not from the species Zea mays.

83. The plant or plant part according to any of statements 55 to 72, wherein said plant or plant part is not from the species Triticum urartu.

84. The plant or plant part according to any of statements 54 to 83, wherein said plant part is not propagation material.

85. The plant or plant part according to any of statements 54 to 84, wherein said plant part is a plant organ, tissue, or cell.

86. The plant or plant part according to statement 85, wherein said plant part is seed, pollen, oocyte, protoplast, inflorescence, embryo, or callus.

87. The plant or plant part according to any of statements 55 to 86, which is mutagenized.

88. The plant or plant part according to any of statements 55 to 86, which is transgenic or gene-edited.

89. Use of a plant according to any of statements 54 to 88 for generating hybrid plants or plant parts.

90. A method for generating hybrid plants or plant parts comprising crossing a first plant according to any of statements 54 to 88 with a second plant, and harvesting seeds.

91. The method according to statement 90, wherein said first plant and said second plant are from the same genus.

92. The method according to statement 90 or 91, wherein said first plant and said second plant are from the same species.

93. A method for generating hybrid plants or plant parts comprising crossing a first plant having a homozygous mutation in MYB80 and in DWARF11 with a second plant, and harvesting seeds.

94. The method according to statement 93, wherein said mutation is a nonsense or missense mutation.

95. The method according to statement 93 or 94, wherein said mutation is a frameshift mutation.

96. The method according to any of statements 93 to 95, wherein said mutation is an indel mutation.

97. The method according to any of statements 93 to 96, wherein said mutation is a dominant (negative) mutation or a recessive mutation.

98. The method according to any of statements 93 to 97, wherein said mutation is a knock-out or knock-down mutation.

99. The method according to any of statements 93 to 98, wherein said mutation is in the coding sequence, a splicing signal or affecting a splice signal, or a regulatory element and/or in the first exon.

100. The method according to any of statements 93 to 99, wherein said mutation is introduced by (random) mutagenesis, preferably TILLING.

101. The method according to any of statements 93 to 100, wherein said mutation is introduced by site-directed mutagenesis, preferably, gene-editing, preferably by CRISPR/Cas.

102. The method or plant (part) according to any of the previous statements, wherein the Dwarf11 gene, coding sequence, or protein as referred to herein has, comprises, consists (essentially) of, or is comprised in a sequence which is at least 80%, at least 85%, at least 90%, at least 95%, at least 96%), at least 97%, at least 98%, or at least 99% identical to as set forth in in any of SEQ ID NOs: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 162, 163, 164, 169, 170, 171, 175, 176, 177, 178, 179, 180, 181, 186, 187, 188, 189, 190, 191, 192, 193, 198, 199, 200, 201, 206, 207, 208, 209, 214, 215, 216, 217, 226, 227, 228, 229, 234, 235, 236, 237, 254, 255, 256, 257, 262, 263, 264, 265, 270, 271, 272, 273, 278, 279, 280, 281, 282, 283, 284, 289, 290, 291, 292, 297, 298, 299, 300, 305, 306, 307, 308, 309, 310, 311, 312, 313, 314, 315, 316, 317, 318, 319, 320, 321, 322, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 377, 378, 379, 380, 385, 386, 387, or 388.

103. The method or plant (part) according to any of the previous statements, wherein the Myb80 gene, coding sequence, or protein as referred to herein has, comprises, consists (essentially) of, or is comprised in a sequence which is at least 80%, at least 85%, at least 90%, at least 95%, at least 96%), at least 97%, at least 98%, or at least 99% identical to as set forth in in any of SEQ ID NOs: 152, 153, 154, 155, 156, 157, 158, 159, 160, 161, 165, 166, 167, 168, 172, 173, 174, 182, 183, 184, 185, 194, 195, 196, 197, 202, 203, 204, 205, 210, 211, 212, 213, 218, 219, 220, 221, 222, 223, 224, 225, 230, 231, 232, 233, 238, 239, 240, 241, 242, 243, 244, 245, 246, 247, 248, 249, 250, 251, 252, 253, 258, 259, 260, 261, 266, 267, 268, 269, 274, 275, 276, 277, 285, 286, 287, 288, 293, 294, 295, 296, 301, 302, 303, 304, 323, 324, 325, 326, 327, 328, 329, 330, 331, 332, 333, 334, 373, 374, 375, 376, 381, 382, 383, 384, 389, 390, 391, or 392.

104. A method for developing an assay to (phenotypically) detect a (allele of a) gene of interest in a plant or plant part, comprising screening for the presence of genes located at most 1 Mb up- or downstream in the chromosome comprising said gene of interest, and selecting a gene causing or capable of causing a (allele-dependent) phenotype in a plant or plant part (as a proxy for (phenotypically) detecting said gene of interest).

105. The method according to statement 104, wherein said gene of interest is a gene (at least one allele of which) causing male or female sterility, preferably genetic male or female sterility, preferably genetic male sterility.

106. The method according to statement 104 or 105, wherein said phenotype is a morphological phenotype.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised, and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

The term “plant” includes whole plants, including descendants or progeny thereof. As used herein unless clearly indicated otherwise, the term “plant” intends to mean a plant at any developmental stage. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc. “Parts of a plant” are e.g. shoot vegetative organs/structures, e.g., leaves, stems and tubers; roots, flowers and floral organs/structures, e.g. bracts, sepals, petals, stamens, carpels, anthers and ovules; seed, including embryo, endosperm, and seed coat; fruit and the mature ovary; plant tissue, e.g. vascular tissue, ground tissue, and the like; and cells, e.g. guard cells, egg cells, pollen, trichomes and the like; and progeny of the same. Parts of plants may be attached to or separate from a whole intact plant. Such parts of a plant include, but are not limited to, organs, tissues, and cells of a plant, and preferably seeds. A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. “Plant cell culture” means cultures of plant units such as, for example, protoplasts, cell culture cells, cells in plant tissues, pollen, pollen tubes, ovules, embryo sacs, zygotes and embryos at various stages of development. “Plant material” refers to leaves, stems, roots, flowers or flower parts, fruits, pollen, egg cells, zygotes, seeds, cuttings, cell or tissue cultures, or any other part or product of a plant. This also includes callus or callus tissue as well as extracts (such as extracts from taproots) or samples. A “plant organ” is a distinct and visibly structured and differentiated part of a plant such as a root, stem, leaf, flower bud, or embryo. “Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue. In certain embodiments, the plant part is a plant organ, tissue, or cell. In certain embodiments, the plant part is seed, pollen, oocyte, protoplast, inflorescence, embryo, or callus.

In certain embodiments, the plant part or derivative is (functional) propagation material, such as germplasm, a seed, or plant embryo or other material from which a plant can be regenerated. In certain embodiments, the plant part or derivative comprises (functional) male and/or female reproductive organs.

In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc.

As used herein unless clearly indicated otherwise, the term “plant” intended to mean a plant at any developmental stage.

In certain embodiments, the plant is a crop plant, such as a cash crop, fodder crop, or subsistence crop, such as food or non-food crops, including agriculture, horticulture, floriculture, or industrial crops. The term crop plant has its ordinary meaning as known in the art. By means of further guidance, and without limitation, a crop plant is a plant grown by humans for human or animal food and other resources, and can be grown and harvested extensively for profit or subsistence, typically in an agricultural setting or context. In certain embodiments, the plant or plant part is from a crop plant of the Poaceae family, or of the Pooideae, Panicoideae, Chloridoideae, Pharoideae, Bambusoideae, or Oryzoideae subfamily. In certain embodiments, the plant is a turf grass.

As used herein, the term Poaceae refers to the family of grasses, or Gramineae. Preferably, the Poaceae are cereals (or cereal grasses), which are in particular cultivated for the edible components of its grain, fodder grasses, or turf grasses. In certain embodiments, the plants or plant parts are from the family of Poaceae. In certain embodiments, the plants or plant parts are from the subfamily of Pooideae. In certain embodiments, the plants or plant parts are from the subfamily of Panicoideae. In certain embodiments, the plants or plant parts are from the subfamily of Oryzoideae. In certain embodiments, the plants or plant parts are from the subfamily of Chloridoideae. In certain embodiments, the plants or plant parts are from the subfamily of Pharoideae. In certain embodiments, the plants or plant parts are from the subfamily of Bambusoideae.

As used herein, the term “plant (part) population” may be used interchangeably with population of plants or plant parts. A plant (part) population preferably comprises a multitude of individual plants (or plant parts thereof), such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

In certain embodiments, the plant population (or plant parts thereof) is a plant line, strain, or variety. In certain embodiments, the plant population (or plant parts thereof) is not a plant line, strain, or variety. In certain embodiments, the plant population (or plant parts thereof) is an inbred plant line, strain, or variety. In certain embodiments, the plant population (or plant parts thereof) is not an inbred plant line, strain, or variety. In certain embodiments, the plant population (or plant parts thereof) is an outbred plant line, strain, or variety. In certain embodiments, the plant population (or plant parts thereof) is not an outbred plant line, strain, or variety.

As used herein, the terms “progeny” and “progeny plant” refer to a plant generated from vegetative or sexual reproduction from one or more parent plants. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1 s, F2 s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids. The term “progeny” can in certain embodiments be used interchangeably with “offspring”, in particular when the plant or plant material is derived from sexual crossing of parent plants.

Preferably, in crosses between a first plant and a second plant, both plants are from the same genus, preferably from the same species. However, interspecies crosses are also possible.

The term “locus” (loci plural) means a specific place or places or a site on a chromosome where for example a QTL, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question.

An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window. A QTL may encode for one or more alleles that affect the expressivity of a continuously distributed (quantitative) phenotype. In certain embodiments, the QTL as described herein may be homozygous. In certain embodiments, the QTL as described herein may be heterozygous.

As used herein, the term “allele” or “alleles” refers to one or more alternative forms, i.e. different nucleotides or nucleotide sequences, of a locus, such as a gene, marker, QTL, etc.

As used herein, the term “mutant alleles” or “mutation” of alleles include alleles having one or more mutations, such as insertions, deletions, stop codons, base changes (e.g., transitions or transversions), or alterations in splice junctions, which may or may not give rise to altered gene products. Modifications in alleles may arise in coding or non-coding regions (e.g. promoter regions, exons, introns or splice junctions).

The term “mutation” or “mutated” as used herein refers to a gene or protein product thereof which is altered or modified such that the function normally attributed to the gene or protein product thereof is altered, or alternatively such that the expression, stability, and/or activity normally associated with the gene or protein product thereof is altered. Typically, a mutation as referred to herein results in a phenotypic effect, such as male sterility or dwarfism, as described herein elsewhere. It will be understood that a mutation in a gene or protein product thereof is referred to in comparison with a gene or protein product thereof not having such mutation, such as a wild type or endogenous gene or protein product thereof. Typically, a mutation refers to a modification at the DNA level, and includes changes in the genetics and/or epigenetics. An alteration in the genetics may include an insertion, a deletion, an introduction of a stop codon, a base change (e.g. transition or transversion), or an alteration in splice junctions. These alterations may arise in coding or non-coding regions (e.g. promoter regions, exons, introns or splice junctions) of the endogenous DNA sequence. For example, an alteration in the genetics may be the exchange (including insertions, deletions) of at least one nucleotide in the endogenous DNA sequence or in a regulatory sequence of the endogenous DNA sequence. If such a nucleotide exchange takes place in a promoter, for example, this may lead to an altered activity of the promoter, since, for example, cis-regulator elements are modified such that the affinity of a transcription factor to the mutated cis-regulatory elements is altered in comparison to the wild-type promoter, so that the activity of the promoter with the mutated cis-regulatory elements is increased or reduced, depending upon whether the transcription factor is a repressor or inductor, or whether the affinity of the transcription factor to the mutated cis-regulatory elements is intensified or weakened. If such a nucleotide exchange occurs, e.g., in a coding region of the endogenous DNA sequence, this may lead to an amino acid exchange in the encoded protein, which may produce an alteration in the activity or stability of the protein, in comparison to the wild-type protein. An alteration in the epigenetics may take place via an altered methylation pattern of the DNA. In certain embodiments, a mutation as referred to herein relates to the insertion of one or more nucleotides in a gene. In certain embodiments, a mutation as referred to herein relates to the deletion of one or more nucleotides in a gene. In certain embodiments, the mutation as referred to herein relates to the deletion as well as the insertion of one or more nucleotides. In certain embodiments, certain nucleotide stretches, such as for instance encoding a particular protein domain are deleted. In certain embodiments, certain nucleotide stretches, such as for instance encoding a particular protein domain are deleted and replaced by nucleotide sequences encoding a different protein domain. In certain embodiments, a mutation as referred to herein relates to the exchange of one or more nucleotides in a gene by different nucleotides. In certain embodiments, the mutation is a nonsense mutation (i.e. the mutation results in the generation of a stop codon in a protein encoding sequence). In certain embodiments, the mutation is a frameshift mutation (i.e. an insertion or deletion of one or more nucleotides (not equal to three or a product thereof) in a protein encoding sequence). In certain embodiments, the mutation results in a truncated protein product. In certain embodiments, the mutation results in an N-terminally truncated protein product. In certain embodiments, the mutation results in a C-terminally truncated protein product. In certain embodiments, the mutation results in an N-terminally and C-terminally truncated protein product. In certain embodiments, the mutation results in an altered splice site (such as an altered splice donor and/or splice acceptor site). In certain embodiments, the mutation is in an exon. In certain embodiments, the mutation is in an intron. In certain embodiments, the mutation is in a regulatory sequence, such as a promoter. In certain embodiments, the mutation results in a codon encoding a different amino acid. In certain embodiments, the mutation results in the insertion or deletion of one or more codons (i.e. nucleotide triplets). In certain embodiments, the mutation results in gene deletion. In certain embodiments, the mutation is a knockout mutation. Both frameshift and nonsense mutations can in certain embodiments be considered as knockout mutations, in particular if the mutation is present in an early exon. A knockout mutation (or loss of function mutation) as used herein preferably means that a functional gene product, such as a functional protein, is not produced anymore. In particular, frameshift and nonsense mutations will lead to premature termination of protein translation, such that a truncated protein will result, which often lacks the required stability and/or activity to perform the function naturally attributed to it. In certain embodiments, the mutation is a knockdown mutation. Knockout mutations often, and according to the present invention preferably are recessive. In contrast to a knockout mutation, a knockdown mutation results in a decreased activity, stability, and/or expression (rate) of the native functional gene product, such as a protein, and thereby ultimately in a decreased functionality. For instance, mutations in promoter regions affecting transcriptional activator binding (or other regulatory sequences), in particular reducing transcription rate, can be considered knockdown mutations. Also mutations negatively affecting protein stability (such as to increase ubiquitination and subsequent protein degradation) can be considered knockdown mutations). In addition, mutations negatively affecting protein activity (such as binding strength or enzymatic activity) can be considered knockdown mutations. While mutation described herein may be non-naturally occurring, this need not necessarily be the case. In certain embodiments, the term “mutated Myb80 protein” or “mutated Dwarf11 protein” can be used interchangeably respectively with “male sterility (Myb80) protein” or “dwarfism (Dwarf11) protein” or the like. As used herein, a mutated Myb80 or Dwarf11 protein, gene, allele, or coding sequence (i.e. polynucleic acid encoding for instance a protein) can be used interchangeably with a protein, gene, allele, or coding sequence conferring respectively (genetic) male sterility or dwarfism, as described herein elsewhere. It will be understood that preferably, the mutation as described herein, such as the Myb80 mutation or the Dwarf11 mutation, results in the expression, activity, and/or stability of the gene product (i.e. mRNA and/or protein) being absent or reduced, as described herein elsewhere. It will be further understood that depending of the zygosity of the mutated gene, a phenotypic effect may or may not occur. As is known in the art, recessive mutations only result in a phenotype if present homozygously, as may for instance be the case for the Myb80 and Dwarf11 mutations as described herein. Accordingly, reference herein to a mutation leading to absent or reduced expression, activity, and/or stability of a gene product may relate to expression, activity, and/or stability resulting from a single allele. A phenotypic effect may arise in a heterozygous state (in case of a dominant allele) or may only arise in a homozygous state (in case of a recessive allele). In case of the Myb80 and Dwarf11 genes (or any other gene pair having recessive mutations), reference to a mutation causing genetic male sterility and dwarfism respectively, indicates that such phenotypes manifest only in case the mutation is homozygous, notwithstanding being referred to as “dwarfism causing mutation” or “genetic male sterility causing mutation”. Such references indicate the capacity to result in dwarfism or genetic male sterility in a homozygous state.

In certain embodiments, the mutation as referred to herein is homozygous. In certain embodiments, the mutation as referred to herein is heterozygous. In certain embodiments, the mutation of both genes in the gene pair as referred to herein is homozygous. In certain embodiments, the mutation of both genes in the gene pair as referred to herein is heterozygous. In certain embodiments, the Myb80 mutation as referred to herein is homozygous. In certain embodiments, the Myb80 mutation as referred to herein is heterozygous. In certain embodiments, the Dwarf11 mutation as referred to herein is homozygous. In certain embodiments, the Dwarf11 mutation as referred to herein is heterozygous. In certain embodiments, the Myb80 and Dwarf11 mutation as referred to herein is homozygous. In certain embodiments, the Myb80 and Dwarf11 mutation as referred to herein is heterozygous.

Preferably, as used herein, a mutation in a gene includes mutations in regulatory sequences of such gene, such as in particular the promoter. Mutations in promoter sequences in particular, and preferably according to the present invention, may alter, disturb, attenuate, reduce, or eliminate transcription.

In certain embodiments, a wild type/endogenous allele is replaced by a mutated allele, preferably all wild type/endogenous alleles are replaced by a mutated allele. Replacement can be effected by any means known in the art, as also described herein elsewhere. Replacement, as used herein also includes (direct) mutagenesis of the wild type/endogenous allele(s) at its native genomic locus. Accordingly, in certain embodiments, a wild type/endogenous allele is mutated, as described herein elsewhere, optionally all wild type/endogenous alleles are mutated. The skilled person will understand that only one copy of a wild type/endogenous allele may be mutated and that homozygosity (if so desired) may be obtained by selfing and subsequent selection. In certain embodiments, a reduced number of wild type/endogenous alleles is present (i.e. the wild type/endogenous allele is heterozygous).

According to the invention, mutations as described herein may be constitutive or conditional or inducible, and/or may be (multiple or single) tissue, organ, or cell (type) specific. The skilled person has ample knowledge how to implement such (differential) mutagenesis.

As used herein, “reducing the expression (rate)” or “reduction in the expression (rate)” or “suppression of the expression” “reduced expression (rate)” or “repression” or a comparable phrase in certain embodiments means a reduction in the expression level or rate of a nucleotide or protein sequence by more than 10%, 15%, 20%, 25% or 30%, preferably by more than 40%, 45%, 50%, 55%, 60% or 65%, more preferably by more than 70%, 75%, 80%, 85%, 90%, 92%, 94%, 96% or 98% in comparison to the specified reference or threshold, such as a plant not comprising the genetic or phenotypic or otherwise modifications according to the invention as described herein elsewhere, or a reference plant (e.g. a genetic male fertile plant or a plant not having dwarfism). However, it may also mean that the expression (rate) of a nucleotide sequence or protein is reduced by 100%. The reduction in the expression (rate) preferably leads to a change of the phenotype of a plant in which the expression (rate) is reduced. In the context of the present invention, an altered phenotype may be genetic male sterility in the case of Myb80 or dwarfism in case of Dwarf11.

“Reduction in the transcription rate” or “reduced transcription rate” or a comparable phrase in certain embodiments means a reduction in the transcription rate of a nucleotide sequence by more than 10%, 15%, 20%, 25% or 30%, preferably by more than 40%, 45%, 50%, 55%, 60% or 65%, more preferably by more than 70%, 75%, 80%, 85%, 90%, 92%, 94%, 96% or 98% in comparison to the specified reference or threshold, such as a plant not comprising the genetic or phenotypic or otherwise modifications according to the invention as described herein elsewhere, or a reference plant. However, it may also mean that the transcription rate of a nucleotide sequence is reduced by 100%. The reduction in the transcription rate preferably leads to a change of the phenotype of a plant in which the transcription rate is reduced. In the context of the present invention, an altered phenotype may be genetic male sterility in the case of Myb80 or dwarfism in case of Dwarf11.

As used herein, “reduced (protein) activity” refers to reduced activity of about at least 10%, preferably at least 30%, more preferably at least 50%, such as at least 20%, 40%, 60%, 80% or more, such as at least 85%, at least 90%, at least 95%, or more. Activity is (substantially) absent or eliminated if activity is reduced at least 80%, preferably at least 90%, more preferably at least 95%. In certain embodiments, activity is (substantially) absent, if no activity, in particular the wild type or native protein activity, can be detected. (Protein) activity levels can be determined by any means known in the art, depending on the type of protein, such as by standard detection methods, including for instance enzymatic assays (for enzymes), transcription assays (for transcription factors), assays to analyse a phenotypic output, etc. Activity may be compared to a reference or threshold as defined above.

As used herein, “reduced stability” may refer to reduced protein stability or reduced RNA, such as mRNA stability. Stability of proteins or RNA can be determined by means known in the art, such as determination of protein/RNA half-life. Reduced protein or RNA stability in certain embodiments means a reduction of stability of about at least 10%, preferably at least 30%, more preferably at least 50%, such as at least 20%, 40%, 60%, 80% or more, such as at least 85%, at least 90%, or at least 95. Stability may be compared to a reference or threshold as defined above.

The skilled person will understand that a reduction in expression, transcription, activity, and/or stability is evaluated compared to a reference, as indicated above, in a comparable context, preferably in the same context, such as a particular developmental stage and/or a particular organ, tissue, or cell type.

As used herein, the terms “not expressing” or “lacking expression” (and mutatis mutandis for instance “lacking activity”) refer to plants or plant part not capable of expression, e.g. by knockout mutations or gene deletions and the like. It will be understood that such absence of expression refers to expression in cells, tissues, or organs in which otherwise (i.e. in case no mutation is present) expression would occur.

It will be understood that reduced or absent expression, activity, and/or stability refers to expression, activity, and/or stability of the wild type, functional, or full length gene product. The skilled person will understand that not every polymorphism will result in a phenotypic change. In such case, even a gene (allele) comprising such polymorphisms may still be considered wild type, functional, or full length. It will be understood that reduced or absent expression, activity, and/or stability results in or is capable of resulting in a phenotype (such as dwarfism or genetic male sterility), even if recessive, and hence only present if the mutation(s) is (are) homozygous.

Mutations as described herein may be introduced by mutagenesis, which may be performed in accordance with any of the techniques known in the art. As used herein, “mutagenization” or “mutagenesis” includes both conventional mutagenesis and location-specific mutagenesis or “genome editing” or “gene editing”. In conventional mutagenesis, modification at the DNA level is not produced in a targeted manner. The plant cell or the plant is exposed to mutagenic conditions, such as TILLING, via UV light exposure or the use of chemical substances (Till et al., 2004). An additional method of random mutagenesis is mutagenesis with the aid of a transposon. Location-specific mutagenesis enables the introduction of modification at the DNA level in a target-oriented manner at predefined locations in the DNA. For example, TALENS, meganucleases, homing endonucleases, zinc finger nucleases, or a CRISPR/Cas system as further described herein may be used for this.

In certain embodiments, a wild type/endogenous allele is knocked out, optionally all wild type/endogenous alleles are knocked out, and a mutated allele is transgenically introduced, transiently or genomically integrated, preferably genomically integrated. In certain embodiments, a wild type/endogenous allele is knocked out, optionally all wild type/endogenous alleles are knocked out, and is transgenically replaced by a mutated allele (at the native genomic location of the wild type allele). The skilled person will understand that only one copy of a wild type/endogenous allele may be knocked out and that homozygosity (if so desired) may be obtained by selfing and subsequent selection.

In certain embodiments, the mutations as described herein, such as the Myb80 mutations or the Dwarf11 mutations, are or result in amino acid substitutions (compared to the wild type or unmutated protein, gene, or coding sequence). In certain embodiments, the mutation is a point mutation. Preferably, the mutation is a missense mutation (i.e. the mutation results in a codon encoding a different amino acid). In certain embodiments one or more mutations are present. In certain embodiments, from 1 to 10 mutations are present. In certain embodiments, from 1 to 9 mutations are present. In certain embodiments, from 1 to 8 mutations are present. In certain embodiments, from 1 to 7 mutations are present. In certain embodiments, from 1 to 6 mutations are present. In certain embodiments, from 1 to 5 mutations are present. In certain embodiments, from 1 to 4 mutations are present. In certain embodiments, from 1 to 3 mutations are present. In certain embodiments, from 1 to 2 mutations are present. In certain embodiments, 1 mutation is present. In certain embodiments, from 1 to 10 amino acid substitutions are present in the mutated protein. In certain embodiments, from 1 to 9 amino acid substitutions are present in the mutated protein. In certain embodiments, from 1 to 8 amino acid substitutions are present in the mutated protein. In certain embodiments, from 1 to 7 amino acid substitutions are present in the mutated protein. In certain embodiments, from 1 to 6 amino acid substitutions are present in the mutated protein. In certain embodiments, from 1 to 5 amino acid substitutions are present in the mutated protein. In certain embodiments, from 1 to 4 amino acid substitutions are present in the mutated protein. In certain embodiments, from 1 to 3 amino acid substitutions are present in the mutated protein. In certain embodiments, from 1 to 2 amino acid substitutions are present in the mutated protein. In certain embodiments, 1 amino acid substitution is present in the mutated protein. In certain embodiments, from 1 to 10 point mutations, preferably missense mutations, are present in the mutated gene, allele, or coding sequence. In certain embodiments, from 1 to 9 point mutations, preferably missense mutations, are present in the mutated gene, allele, or coding sequence. In certain embodiments, from 1 to 8 point mutations, preferably missense mutations, are present in the mutated gene, allele, or coding sequence. In certain embodiments, from 1 to 7 point mutations, preferably missense mutations, are present in the mutated gene, allele, or coding sequence. In certain embodiments, from 1 to 6 point mutations, preferably missense mutations, are present in the mutated gene, allele, or coding sequence. In certain embodiments, from 1 to 5 point mutations, preferably missense mutations, are present in the mutated gene, allele, or coding sequence. In certain embodiments, from 1 to 4 point mutations, preferably missense mutations, are present in the mutated gene, allele, or coding sequence. In certain embodiments, from 1 to 3 point mutations, preferably missense mutations, are present in the mutated gene, allele, or coding sequence. In certain embodiments, from 1 to 2 point mutations, preferably missense mutations, are present in the mutated gene, allele, or coding sequence. In certain embodiments, 1 point mutation, preferably missense mutation, is present in the mutated gene, allele, or coding sequence.

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A genetic element, an introgression fragment, a QTL or a gene or allele conferring a trait is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only plants comprising the genetic element, locus, introgression fragment, gene or allele can be used, but also progeny/descendants from such plants which have been selected to retain the genetic element, locus, introgression fragment, gene or allele, can be used and are encompassed herein. Whether a plant (or genomic DNA, cell or tissue of a plant) comprises the same genetic element, locus, introgression fragment, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

“Introducing” in the meaning of the present invention includes stable or transient integration by means of transformation including Agrobacterium-mediated transformation, transfection, microinjection, biolistic bombardment, insertion using gene editing technology like CRISPR systems (e.g. CRISPR/Cas, in particular CRISPR/Cas9 or CRISPR/Cas12), CRISPR/CasX, or CRISPR/CasY), TALENs, zinc finger nucleases or meganucleases, homologous recombination optionally by means of one of the below mentioned gene editing technology including preferably a repair template, modification of endogenous gene using random or targeted mutagenesis like TILLING or above mentioned gene editing technology, etc.

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). The term “transgenic” here means genetically modified by the introduction of a non-endogenous nucleic acid sequence. Typically a species-specific nucleic acid sequence is introduced in a form, arrangement or quantity into the cell in a location where the nucleic acid sequence does not occur naturally in the cell. This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene”, “exogene”, or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Gene editing” or “genome editing” refers to genetic engineering in which DNA or RNA is inserted, deleted, modified or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically induce a mutation in for instance the Myb80 and/or Dwarf11 gene, as described herein elsewhere, such as to generate a mutation or a knockout of the gene. Alternatively, by means of for instance RNA-specific CRISPR/Cas systems, a knockdown can be achieved, as RNA/specific CRISPR/Cas systems (such as Cas13) allow site-directed cleavage of (single-stranded) RNA. Accordingly, in certain embodiments, designer nucleases, in particular RNA-specific CRISPR/Cas systems are developed which specifically target the mRNA, such as to cleave mRNA and generate a knockdown of the gene/mRNA/protein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues, i.e. a process in which a single-stranded nucleic acid molecule attaches itself to a complementary nucleic acid strand, i.e. agrees with this base pairing. Standard procedures for hybridization are described, for example, in Sambrook et al. (Molecular Cloning. A Laboratory Manual, Cold Spring Harbor Laboratory Press, 3rd edition 2001). The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence. Preferably this will be understood to mean an at least 50%, more preferably at least 55%, 60%, 65%, 70%, 75%, 80% or 85%, more preferably 90%, 91%, 92%, 93%, 94%, 95%, 96%, 97%, 98% or 99% of the bases of the nucleic acid strand form base pairs with the complementary nucleic acid strand. The possibility of such binding depends on the stringency of the hybridization conditions.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template. In certain embodiments, the QTL allele of the invention as described herein may be provided on/as a template. By designing the system such that double strand breaks are introduced upstream and downstream of the corresponding region in the genome of a plant, this region is excised and can be replaced with the template comprising a mutated gene of the invention. In this way, introduction of the mutated gene (allele) of the invention in a plant need not involve multiple backcrossing, in particular in a plant of specific genetic background. Similarly, the polynucleic acid of the invention may be provided on/as a template. More advantageously however, the polynucleic acid of the invention may be generated without the use of a recombination template, but solely through the endonuclease action leading to a double strand DNA break which is repaired by NHEJ, resulting in the generation of indels.

In certain embodiments, the nucleic acid modification or mutation is effected by a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39: e82; Zhang F. Cong L. Lodato S. Kosuri S. Church GM. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35) z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. No. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. With respect to general information on CRISPR/Cas Systems, components thereof, and delivery of such components, including methods, materials, delivery vehicles, vectors, particles, and making and using thereof, including as to amounts and formulations, as well as Cas9CRISPR/Cas-expressing eukaryotic cells, Cas-9 CRISPR/Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to US provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014. Mention is also made of U.S. application 62/180,709, 17-Jun-15, PROTECTED GUIDE RNAS (PGRNAS); U.S. application 62/091,455, filed, 12-Dec-14, PROTECTED GUIDE RNAS (PGRNAS); U.S. application 62/096,708, 24-Dec-14, PROTECTED GUIDE RNAS (PGRNAS); U.S. applications 62/091,462, 12-Dec-14, 62/096,324, 23-Dec-14, 62/180,681, 17 Jun. 2015, and 62/237,496, 5 Oct. 2015, DEAD GUIDES FOR CRISPR TRANSCRIPTION FACTORS; U.S. application 62/091,456, 12-Dec-14 and 62/180,692, 17 Jun. 2015, ESCORTED AND FUNCTIONALIZED GUIDES FOR CRISPR-CAS SYSTEMS; U.S. application 62/091,461, 12-Dec-14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19-Dec-14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24-Dec-14, ENGINEERING OF SYSTEMS, METHODS AND OPTIMIZED ENZYME AND GUIDE SCAFFOLDS FOR SEQUENCE MANIPULATION; U.S. application 62/098,059, 30-Dec-14, 62/181,641, 18 Jun. 2015, and 62/181,667, 18 Jun. 2015, RNA-TARGETING SYSTEM; U.S. application 62/096,656, 24-Dec-14 and 62/181,151, 17 Jun. 2015, CRISPR HAVING OR ASSOCIATED WITH DESTABILIZATION DOMAINS; U.S. application 62/096,697, 24-Dec-14, CRISPR HAVING OR ASSOCIATED WITH AAV; U.S. application 62/098,158, 30-Dec-14, ENGINEERED CRISPR COMPLEX INSERTIONAL TARGETING SYSTEMS; U.S. application 62/151,052, 22-Apr-15, CELLULAR TARGETING FOR EXTRACELLULAR EXOSOMAL REPORTING; U.S. application 62/054,490, 24-Sep-14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; US application 61/939,154, 12-F EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25-Sep-14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4-Dec-14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24-Sep-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23-Oct-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24-Sep-14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24-Sep-14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25-Sep-14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25-Sep-14, MULTIFUNCTIONAL-CRISPR COMPLEXES AND/OR OPTIMIZED ENZYME LINKED FUNCTIONAL-CRISPR COMPLEXES; U.S. application 62/087,475, 4-Dec-14 and 62/181,690, 18 Jun. 2015, FUNCTIONAL SCREENING WITH OPTIMIZED FUNCTIONAL CRISPR-CAS SYSTEMS; U.S. application 62/055,487, 25-Sep-14, FUNCTIONAL SCREENING WITH OPTIMIZED FUNCTIONAL CRISPR-CAS SYSTEMS; U.S. application 62/087,546, 4-Dec-14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30-Dec-14, CRISPR MEDIATED IN VIVO MODELING AND GENETIC SCREENING OF TUMOR GROWTH AND METASTASIS. Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, and Attorney Docket No. 46783.01.2128, filed 22 Oct. 2015, NOVEL CRISPR ENZYMES AND SYSTEMS, U.S. application 62/232,067, 24 Sep. 2015, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12-Dec-2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES. European patent application EP3009511. Reference is further made to Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science Feb 15; 339 (6121): 819-23 (2013); RNA-guided editing of bacterial genomes using CRISPR-Cas systems. Jiang W., Bikard D., Cox D., Zhang F, Marraffini LA. Nat Biotechnol Mar; 31 (3): 233-9 (2013); One-Step Generation of Mice Carrying Mutations in Multiple Genes by CRISPR/Cas-Mediated Genome Engineering. Wang H., Yang H., Shivalila CS., Dawlaty MM., Cheng A W., Zhang F., Jaenisch R. Cell May 9; 153 (4): 910-8 (2013); Optical control of mammalian endogenous transcription and epigenetic states. Konermann S, Brigham MD, Trevino AE, Hsu PD, Heidenreich M, Cong L, Platt RJ, Scott D A, Church GM, Zhang F. Nature. 2013 Aug. 22; 500 (7463): 472-6. doi: 10.1038/Nature 12466. Epub 2013 Aug. 23; Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674 (13) 01015-5. (2013); DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013); Genome engineering using the CRISPR-Cas9 system. Ran, F A., Hsu, P D., Wright, J., Agarwala, V., Scott, D A., Zhang, F. Nature Protocols Nov; 8 (11): 2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, JG., Zhang, F. Science December 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27. (2014). 156 (5): 935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp PA. Nat Biotechnol. (2014) April 20. doi: 10.1038/nbt.2889; CRISPR-Cas9 Knockin Mice for Genome Editing and Cancer Modeling, Platt et al., Cell 159 (2): 440-455 (2014) DOI: 10.1016/j.cell.2014.09.014; Development and Applications of CRISPR-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 Jan. 3; 343 (6166): 80-84. doi:10.1126/science. 1246981; Rational design of highly active sgRNAs for CRISPR-Cas9-mediated gene inactivation, Doench et al., Nature Biotechnology 32 (12): 1262-7 (2014) published online 3 Sep. 2014; doi:10.1038/nbt.3026, and In vivo interrogation of gene function in the mammalian brain using CRISPR-Cas9, Swiech et al, Nature Biotechnology 33, 102-106 (2015) published online 19 Oct. 2014; doi:10.1038/nbt.3055, Cpf1 Is a Single RNA-Guided Endonuclease of a Class 2 CRISPR-Cas System, Zetsche et al., Cell 163, 1-13 (2015); Discovery and Functional Characterization of Diverse Class 2 CRISPR-Cas Systems, Shmakov et al., Mol Cell 60 (3): 385-397 (2015); C2c2 is a single-component programmable RNA-guided RNA-targeting CRISPR effector, Abudayyeh et al, Science (2016) published online Jun. 2, 2016 doi: 10.1126/science.aaf5573. Each of these publications, patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cpf1).

As used herein, the term “crRNA” or “guide RNA” or “single guide RNA” or “sgRNA” or “one or more nucleic acid components” of a CRISPR/Cas locus effector protein, as applicable, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay.

A guide sequence, and hence a nucleic acid-targeting guide RNA may be selected to target any target nucleic acid sequence. The target sequence may be DNA. The target sequence may be genomic DNA. The target sequence may be mitochondrial DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the gRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In particular embodiments, the CRISPR/Cas system requires a tracrRNA. The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and gRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence. In alternative embodiments, the CRISPR/Cas system does not require a tracrRNA, as is known by the skilled person.

In certain embodiments, the guide RNA (capable of guiding Cas to a target locus) may comprise (1) a guide sequence capable of hybridizing to a target locus and (2) a tracr mate or direct repeat sequence (in 5′ to 3′ orientation, or alternatively in 3′ to 5′ orientation, depending on the type of Cas protein, as is known by the skilled person). In particular embodiments, the CRISPR/Cas protein is characterized in that it makes use of a guide RNA comprising a guide sequence capable of hybridizing to a target locus and a direct repeat sequence, and does not require a tracrRNA. In particular embodiments, where the CRISPR/Cas protein is characterized in that it makes use of a tracrRNA, the guide sequence, tracr mate, and tracr sequence may reside in a single RNA, i.e. an sgRNA (arranged in a 5′ to 3′ orientation or alternatively arranged in a 3′ to 5′ orientation), or the tracr RNA may be a different RNA than the RNA containing the guide and tracr mate sequence. In these embodiments, the tracr hybridizes to the tracr mate sequence and directs the CRISPR/Cas complex to the target sequence.

Typically, in the context of an endogenous nucleic acid-targeting system, formation of a nucleic acid-targeting complex (comprising a guide RNA hybridized to a target sequence and complexed with one or more nucleic acid-targeting effector proteins) results in modification (such as cleavage) of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest). The skilled person will be aware of specific cut sites for selected CRISPR/Cas systems, relative to the target sequence, which as is known in the art may be within the target sequence or alternatively 3′ or 5′ of the target sequence.

In some embodiments, the unmodified nucleic acid-targeting effector protein may have nucleic acid cleavage activity. In some embodiments, the nuclease as described herein may direct cleavage of one or both nucleic acid (DNA, RNA, or hybrids, which may be single or double stranded) strands at the location of or near a target sequence, such as within the target sequence and/or within the complement of the target sequence or at sequences associated with the target sequence. In some embodiments, the nucleic acid-targeting effector protein may direct cleavage of one or both DNA or RNA strands within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt (e.g. for Cas9, such as SaCas9 or SpCas9). In some embodiments, the cleavage may be staggered (e.g. for Cpf1), i.e. generating sticky ends. In some embodiments, the cleavage is a staggered cut with a 5′ overhang. In some embodiments, the cleavage is a staggered cut with a 5′ overhang of 1 to 5 nucleotides, preferably of 4 or 5 nucleotides. In some embodiments, the cleavage site is upstream of the PAM. In some embodiments, the cleavage site is downstream of the PAM. In some embodiments, the nucleic acid-targeting effector protein that may be mutated with respect to a corresponding wild-type enzyme such that the mutated nucleic acid-targeting effector protein lacks the ability to cleave one or both DNA or RNA strands of a target polynucleotide containing a target sequence. As a further example, two or more catalytic domains of a Cas protein (e.g. RuvC I, RuvC II, and RuvC III or the HNH domain of a Cas9 protein) may be mutated to produce a mutated Cas protein substantially lacking all DNA cleavage activity. In some embodiments, a nucleic acid-targeting effector protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “modified” Cas generally refers to a Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein.

In certain embodiments, the target sequence should be associated with a PAM (protospacer adjacent motif) or PFS (protospacer flanking sequence or site); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme. Further, engineering of the PAM Interacting (PI) domain may allow programing of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver B P et al. Engineered CRISPR-Cas9 nucleases with altered PAM specificities. Nature. 2015 Jul. 23; 523 (7561): 481-5. doi: 10.1038/nature14592. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. The skilled person will understand that other Cas proteins may be modified analogously.

The Cas protein as referred to herein, such as without limitation Cas9, Cpf1 (Cas12a), C2c1 (Cas12b), C2c2 (Cas13a), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is (modified) Cpf1, preferably Acidaminococcus sp., such as Acidaminococcus sp. BV3L6 Cpf1 (AsCpf1) or Lachnospiraceae bacterium Cpf1, such as Lachnospiraceae bacterium MA2020 or Lachnospiraceae bacterium MD2006 (LbCpf1). In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b.

Further, gene editing may comprise also the exchange of single nucleotides by means of base editors. A base editor as used herein refers to a protein or a fragment thereof having the capacity to mediate a targeted base modification, i.e., the conversion of a base of interest resulting in a point mutation of interest. Preferably, the at least one base editor in the context of the present invention is temporarily or permanently fused to at least one DSBI enzyme, or optionally to a component of at least one DSBI. The fusion can be covalent and/or non-covalent. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalysed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al ((2017). Programmable base editing of A⋅T to G⋅C in genomic DNA without DNA cleavage. Nature, 551 (7681), 464.) described adenine base editors (ABEs) that mediate the conversion of A⋅T to G⋅C in genomic DNA.

In certain embodiments, the nucleic acid modification is effected by random mutagenesis. The skilled person will understand that identification and selection of suitable mutations may include appropriate selection assays, such as functional selection assays (including genotypic or phenotypic selection assays). In random mutagenesis, cells or organisms may be exposed to mutagens such as UV, X-ray, or gamma ray radiation or mutagenic chemicals (such as for instance such as ethyl methanesulfonate (EMS), ethylnitrosourea (ENU), or dimethylsulfate (DMS), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled.

A “bubble” forms at the mismatch of the two DNA strands, which is then cleaved by a single stranded nuclease. The products are then separated by size, such as by HPLC. See also McCallum et al. “Targeted screening for induced mutations”; Nat Biotechnol. 2000 April; 18 (4): 455-7 and McCallum et al. “Targeting Induced Local Lesions IN Genomes (TILLING) for plant functional genomics”; Plant Physiol. 2000 June; 123 (2): 439-42, both incorporated by reference in their entirety. By means of further example, and without limitation, the methodologies described in the following publications, incorporated by reference in their entirety, may be adopted according to the present invention, such as in connection with EMS mutagenesis: Till et al. “Discovery of induced point mutations in maize genes by TILLING”; BMC Plant Biol. 2004 Jul. 28; 4:12; and Weil & Monde “Getting the point-mutations in maize” Crop Sci 2007; 47 S60-S67. The skilled person will understand that depending on the mutagen dose (irradiation of chemical) the (average) mutation density can be varied or fixed. In certain embodiments, the random mutagenesis is single nucleotide mutagenesis. In certain embodiments, the random mutagenesis is chemical mutagenesis, preferably EMS mutagenesis.

“RNA interference” or “RNAi” is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference. RNAs are the direct products of genes, and these small RNAs can bind to other specific messenger RNA (mRNA) molecules and either increase or decrease their activity, for example by preventing an mRNA from being translated into a protein. The RNAi pathway is found in many eukaryotes, including animals, and is initiated by the enzyme Dicer, which cleaves long double-stranded RNA (dsRNA) molecules into short double-stranded fragments of about 21 nucleotide siRNAs (small interfering RNAs). Each siRNA is unwound into two single-stranded RNAs (ssRNAs), the passenger strand and the guide strand. The passenger strand is degraded and the guide strand is incorporated into the RNA-induced silencing complex (RISC). Mature miRNAs are structurally similar to siRNAs produced from exogenous dsRNA, but before reaching maturity, miRNAs must first undergo extensive post-transcriptional modification. A miRNA is expressed from a much longer RNA-coding gene as a primary transcript known as a pri-miRNA which is processed, in the cell nucleus, to a 70-nucleotide stem-loop structure called a pre-miRNA by the microprocessor complex. This complex consists of an RNase III enzyme called Drosha and a dsRNA-binding protein DGCR8. The dsRNA portion of this pre-miRNA is bound and cleaved by Dicer to produce the mature miRNA molecule that can be integrated into the RISC complex; thus, miRNA and siRNA share the same downstream cellular machinery. A short hairpin RNA or small hairpin RNA (shRNA/Hairpin Vector) is an artificial RNA molecule with a tight hairpin turn that can be used to silence target gene expression via RNA interference. The most well-studied outcome is post-transcriptional gene silencing, which occurs when the guide strand pairs with a complementary sequence in a messenger RNA molecule and induces cleavage by Argonaute 2 (Ago2), the catalytic component of the RISC. In will be understood that the RNAi molecules can be applied as such to/in the plant, or can be encoded by appropriate vectors, from which the RNAi molecule is expressed. Delivery and expression systems of RNAi molecules, such as siRNAs, shRNAs or miRNAs are well known in the art.

As used herein, the term “homozygote” refers to an individual cell or plant having the same alleles at one or more or all loci. When the term is used with reference to a specific locus or gene, it means at least that locus or gene has the same alleles. Accordingly, for diploid organisms, the two alleles are identical, for tetraploid organisms, the 4 alleles are identical, etc. As used herein, the term “homozygous” means a genetic condition existing when identical alleles reside at corresponding loci on homologous chromosomes. As used herein, the term “heterozygote” refers to an individual cell or plant having different alleles at one or more or all loci. Accordingly, for diploid organisms, the two alleles are not identical, for tetraploid organisms, the 4 alleles are not identical (i.e. at least one allele is different than the other alleles), etc. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the genes, or coding sequences as described herein are heterozygous. In certain embodiments, genes or coding sequence alleles as described herein is/are homozygous. In certain embodiments, the genes or coding sequence alleles as described herein are heterozygous. It will be understood that homozygosity or heterozygosity preferably relates to at least a gene, i.e. the locus comprising the gene (or coding sequence derived thereof). However, more specifically, homozygosity or heterozygosity may equally refer to a particular mutation, such as a mutation described herein. Accordingly, a particular mutation can be considered to be homozygous (i.e. all alleles carry the mutation), whereas for instance the remainder of the gene, coding sequence, or protein may comprise differences between alleles.

In certain embodiments, the mutation as defined herein is homozygous. Accordingly, in diploid plants the two alleles are identical (at least with respect to the particular mutation), in tetraploid plants the four alleles are identical, and in hexaploid plants the six alleles are identical with respect to the mutation or marker. In certain embodiments, the mutation/marker as defined herein is heterozygous. Accordingly, in diploid plants the two alleles are not identical, in tetraploid plants the four alleles are not identical (for instance only one, two, or three alleles comprise the specific mutation/marker), and in hexaploid plants the six alleles are not identical with respect to the mutation or marker (for instance only one, two, three, four or five alleles comprise the specific mutation/marker). Similar considerations apply in case of pseudopolyploid pants.

A “marker” is a (means of finding a position on a) genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype. A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” or “marker allele” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

The development of markers may be based either on quantitative trait loci (QTL) mapping or genome wide association studies (GWAS). A QTL mapping requires two parental lines (that differ genetically). These two types of mapping are referred to as low resolution mapping due to the fact that the identified chromosomal region (from now on called QTL interval) controlling the trait can contain up to several hundreds of genes within an interval size of 5-30 cM, depending on whether the QTL interval is in the telomeric or pericentromeric region. The reason for such a name is that both QTL mapping as well as GWAS generally only use rather a small size of population (100 to 200 lines), thus the size of the discovered chromosomal interval within which the gene exists becomes very large. Therefore, the markers flanking the identified chromosomal region in 100 percent of cases are not diagnostic, which means the marker score generated doesn't necessarily match the observed phenotype in the field. For this issue to be resolved, the mapping resolution needs to be increased, and the resolution can be increased by increasing recombination events during meiosis. To accomplish this, one has to increase the population to thousands of lines plus have different generations of selfing or back crossing that take at least four years (and are very expensive and labor-intensive). In reality, the breeder must perform high resolution mapping of fine mapping in order to have a marker nearer to the gene. In the initial screening, the thousands of individuals are screened only for initial flanking markers identified through low-resolution mapping. Such a screening aims to find recombinants. Recombination events between markers and the gene will allow to narrow down the interval and come closer to the gene. The next step is designing additional markers within the targeted interval. These may range for instance from ten to twenty markers, depending on how long an interval is. Then screen the newly identified recombinants with these 10-20 markers. This will help find the closest marker with the least number of recombination events between it and the gene, keeping in mind that the marker is only close to the gene, not within it, so there is still a possibility that the marker score does not match the observed phenotype. So, as you can see the approach requires a lot of time, efforts, and money should be invested. The use of literature-based markers is another possibility. It is more convenient and time-efficient if the information of the closet marker is already published, otherwise the publication is only beneficial to determine the corresponding chromosomal interval responsible for the trait of interest.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population. With regard to a SNP marker, allele refers to the specific nucleotide base present at that SNP locus in that individual plant. As used herein, reference to markers or marker alleles refers to markers or marker alleles associated with, linked with, or characteristic of the genes, genotypes, or phenotypes as described herein, unless explicitly referred to otherwise. Such markers or marker alleles are typically annotated as “donor” markers or marker alleles.

“Fine-mapping” refers to methods by which the position of a QTL can be determined more accurately (narrowed down) and by which the size of the introgression fragment comprising the QTL is reduced. For example Near Isogenic Lines for the QTL (QTL-NILs) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g. introgression fragment, transgene, polymorphism, mutation, etc), to select plants for the presence of the specific locus or region (introgression fragment, transgene, polymorphism, mutation, etc). For example, a marker allele genetically linked to a QTL or gene as defined herein, can be used to detect and/or select plants comprising the QTL or gene. The closer the genetic linkage of the marker allele to the locus (e.g. about 10 cM, 7 cM, 6 cM, 5 cM, 4 cM, 3 cM, 2 cM, 1 cM, 0.5 cM or less), the less likely it is that the marker is dissociated from the locus through meiotic recombination. Likewise, the closer two markers are linked to each other (e.g. within 10 cM, 7 cM, 5 cM, 4 cM, 3 cM, 2 cM, 1 cM or less) the less likely it is that the two markers will be separated from one another (and the more likely they will co-segregate as a unit). A marker “within 10 cM or within 7 cM or within 5 cM, 3 cM, 2 cM, or 1 cM” of another marker refers to a marker which genetically maps to within the 10 cM or 7 cM or 5 cM, 3 cM, 2 cM, or 1 cM region flanking the marker (i.e. either side of the marker). Similarly, a marker within 10 Mb, 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 10 Mb, 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker). “LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favour the presence of linkage and a LOD score greater than 3.0 is considered evidence for linkage. A LOD score of +3 indicates 1000 to 1 odds that the linkage being observed did not occur by chance.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (CM). One cM corresponds to a recombination frequency of 1%. If no recombinants can be found, the RF is zero and the loci are either extremely close together physically or they are identical. The further apart two loci are, the higher the RF.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

An allele “negatively” correlates with a trait when it is linked to it and when presence of the allele is an indicator that a desired trait or trait form will not occur in a plant comprising the allele. An allele “positively” correlates with a trait when it is linked to it and when presence of the allele is an indicator that the desired trait or trait form will occur in a plant comprising the allele.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “linked” or “closely linked”, in the present application, means that recombination between two linked loci occurs with a frequency of equal to or less than about 10% (i.e., are separated on a genetic map by not more than 10 cM), or preferably less than about 5% (i.e. 5 cM), more preferably less than about 1% (i.e. 1 cM). Put another way, the closely linked loci co-segregate at least 90% (or 95% or 99%) of the time. Marker loci are especially useful with respect to the subject matter of the current disclosure when they demonstrate a significant probability of co-segregation (linkage) with a desired trait. Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

Alternatively, and preferably according to the invention, a gene pair (such as the Myb80 and Dwarf11 genes) are within 1 Mbp from each other (i.e. on the same chromosome, preferably on the same chromosome arm). In other words, the distance between the two genes is at most 1 Mbp. As used herein, the distance refers to the intergenic distance, in particular the distance between the coding sequences (e.g. between stop codon of the most upstream gene and start codon of the most downstream gene or vice versa, depending on the orientation of the genes).

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosomal segment are also “genetically linked”, typically within a genetic recombination distance of less than or equal to 50 cM, e.g., about 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25 cM or less. That is, two genetic elements within a single chromosomal segment undergo recombination during meiosis with each other at a frequency of less than or equal to about 50%, e.g., about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25% or less. “Closely linked” markers display a cross over frequency with a given marker of about 10% or less, e.g., 9%, 8%, 7%, 6%, 5%, 4%, 3%, 2%, 1%, 0.75%, 0.5%, 0.25% or less (the given marker locus is within about 10 cM of a closely linked marker locus, e.g., 9, 8, 7, 6, 5, 4, 3, 2, 1, 0.75, 0.5, 0.25 cM or less of a closely linked marker locus). Put another way, closely linked marker loci co-segregate at least about 90% the time, e.g., 91%, 92%, 93%, 94%, 95%, 96%, 97%, 98%, 99%, 99.5%, 99.75%, or more of the time.

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (BI2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the BI2seq program can be found in the readme file accompanying BLASTZ. BI2seq performs a comparison between two sequences using either the BLASTN or BLASTP algorithm. Preferably, BLAST sequence alignments are performed according to the standard (i.e. default) settings (i.e. at the filing date of the present application).

BLASTN is used to compare nucleic acid sequences, while BLASTP is used to compare amino acid sequences. To compare two nucleic acid sequences, the options are set as follows: −i is set to a file containing the first nucleic acid sequence to be compared (e.g., C:\seq I.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. The following command will generate an output file containing a comparison between two sequences: C:\B12seq −i c:\seql.txt −j c:\seq2.txt −p blastn −o c:\output.txt −q −1 −r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the BI2seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used. The terms “polynucleic acid”, “nucleotide sequence(s)”, “polynucleotide(s)”, “nucleic acid sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

An “isolated nucleic acid sequence”, “isolated polynucleic acid” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When reference is made to a nucleic acid sequence (e.g. DNA or genomic DNA) having “substantial sequence identity to” a reference sequence or having a sequence identity of at least 80%>, e.g. at least 85%, 90%, 95%, 98%> or 99%>nucleic acid sequence identity to a reference sequence, in one embodiment said nucleotide sequence is considered substantially identical to the given nucleotide sequence and can be identified using hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically, stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001). Examples of high stringent hybridization conditions are conditions under which primarily only those nucleic acid molecules that have at least 90% or at least 95% sequence identity undergo hybridization. Such high stringent hybridization conditions are, for example: 4×SSC at 65° C. and subsequent multiple washes in 0.1×SSC at 65° C. for approximately 1 hour. The term “high stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions. Less stringent hybridization conditions are, for example: hybridizing in 4×SSC at 37° C. and subsequent multiple washing in 1×SSC at room temperature.

It will be understood that “specifically hybridizing” means that the polynucleic acid hybridises with the (molecular) marker allele (such as under stringent hybridisation conditions, as defined herein elsewhere), but does not (substantially) hybridise with a polynucleic acid not comprising the marker allele or is (substantially) incapable of being used as a PCR primer. By means of example, in a suitable readout, the hybridization signal with the marker allele or PCR amplification of the marker allele is at least 5 times, preferably at least 10 times stronger or more than the hybridisation signal with a non-marker allele, or any other sequence.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature.

Amino acid substitutions encompass amino acid alterations in which an amino acid is replaced with a different naturally-occurring amino acid residue. Such substitutions may be classified as “conservative<1>, in which an amino acid residue contained in the wild-type protein is replaced with another naturally-occurring amino acid of similar character, for example Gly<→Ala, Val<→Ile<→Leu, Asp<→-Glu, Lys<→Arg, Asn<→Gln or Phe<→Trp<→Tyr. Substitutions encompassed by the present invention may also be “non-conservative”, in which an amino acid residue which is present in the wild-type protein is substituted with an amino acid with different properties, such as a naturally-occurring amino acid from a different group (e.g. substituting a charged or hydrophobic amino acid with alanine. “Similar amino acids”, as used herein, refers to amino acids that have similar amino acid side chains, i.e. amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbours (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine, proline, and tryptophan (all hydrophobic, except for glycine which is neutral).

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

As used herein, the term “endogenous” refers to a gene or allele which is present in its natural genomic location. The term “endogenous” can be used interchangeably with “native” or “wild-type”. This does not however exclude the presence of one or more nucleic acid differences with the wild-type allele. In particular embodiments, the difference with a wild-type allele can be limited to less than 9 preferably less than 6, more particularly less than 3 nucleotide differences, such as 0 nucleotides difference. More particularly, the difference with the wildtype sequence can be in only one nucleotide. Preferably, the endogenous allele encodes a modified protein having less than 9, preferably less than 6, more particularly less than 3 and even more preferably only one or no amino acid difference with the wild-type protein.

A used herein, the term “exogenous polynucleotide” refers to a polynucleotide, such as a gene (or cDNA) or allele which is or has been recombinantly introduced in a cell (or plant). The exogenous polynucleotide may be episomal or genomically integrated. Integration may be random or site-directed. Integration may include replacement of a corresponding endogenous polynucleotide. It will be understood that an exogenous polynucleotide is not naturally present in the cell or plant.

The term “hybrid”, “hybrid plant”, or hybrid seed” as used in the context of the present invention has its ordinary meaning known in the art. By means of further guidance, and without limitation in the context of the present invention this term refers to the offspring of two (genetically distinct or different) parent plants, which may be different plant lines, cultivars, or varieties. It will be understood that according to the present invention, the parents of a hybrid plant preferably are from the same genus, preferably the same species. Preferably, the parents of a hybrid each are stable populations, having a high degree of homozygosity. The parents typically differ from each other in one or more traits or (agronomic, physiologic, or quality) characteristics. The hybrid therefore typically is heterozygous for such trait or (agronomic, physiologic, or quality) characteristic. According to the present invention, hybrids preferably are the F1 hybrids, i.e. the first generation of offspring resulting from the two parents (e.g. the two parental lines, cultivars, or varieties). The seed produced by crossing two parents is therefore the F1 hybrid seed.

As used herein, the term “male sterile” plant (line, cultivar, or variety) has its ordinary meaning in the art. By means of further guidance, and without limitation, the term refers to a plant which is unable to produce offspring as a pollen donor, and may result from the failure to produce (functional) anthers, pollen, or gametes, in particular male gametes. Cytoplasmic male sterile plants have cytoplasmic genes, usually in the mitochondria, that encode factors that disrupt or prevent pollen development, making them male-sterile, with male sterility inherited maternally. The utilization of cytoplasmic male sterility for hybrid seed production requires three separate plant lines: the male-sterile line, an isogeneic male-fertile line for propagation (“maintainer line”) and a line for restoring fertility to the hybrid so that it can produce seed (“restorer line”). The male-sterile line is used as the receptive parent in a hybrid cross, the maintainer line is genetically identical to the male-sterile line, excepting that it lacks the cytoplasmic sterility factors, and the restorer line is any line that masks the cytoplasmic sterility factor. The restorer line is very important for those plants, such as grain sorghum or cotton, the useful crop of which is the seed itself or seed-associated structures. Genetic male sterility is similar to cytoplasmic male sterility but differs in that the sterility factors are encoded in nuclear DNA. Typically, genetic male sterility refers to a change in a plant's genetic structure which results in its ability to produce and/or spread viable pollen. Genetic male sterile plant lines may occur naturally. It is also possible to create a male-sterile plant line (in particular a genetic male sterile plant line) using recombinant techniques. Whether naturally occurring or transgenic, male-sterile lines (in particular genetic male sterile plant lines) still require the use of a sister maintainer line for their propagation, which of necessity leads to a minimum of 50% male-fertile plants in propagated seed. This is a result of the genetics of male-sterility and maintainer lines. If the male-sterility factor is recessive, as most are, a male-sterile plant would have to be homozygous recessive in order to display the trait. Preferably, according to the invention male sterility refers to genetic male sterility. Preferably, according to the invention male sterility is not or does not encompass cytoplasmic male sterility.

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In the case of genetic male or female sterility, a linked gene may confer an easily identifiable phenotype, such as dwarfism, associated with the genetic male or female sterility. Identification of dwarfism can then be used as a proxy for (i.e. in lieu of) identification of genetic male or female sterility.

As used herein, the term “dwarfism” or “dwarf” has its ordinary meaning known in the art. By means of further guidance, and without limitation, the term dwarfism/dwarf/etc. refers to plants or plant parts having a reduced size compared to plant not having dwarfism. The skilled person will understand that a reduced size preferably refers to an average reduced size, i.e. based on a population of plants or plant parts. Due to natural variation (for instance also subject to weather or climatological conditions, as well as geographical conditions) by chance a small plant lacking dwarfism-causing genes or mutations may be smaller than a tall plant comprising dwarfism-causing genes or mutations. However, the selection of small plants or plant parts will in any case result in a large proportion of plants or plant parts having dwarfism-causing genes or mutations. Accordingly, selection of for instance the 25% smallest plants or plant parts (e.g. resulting from a cross between heterozygous Dwarf11 plants) will ensure selection or enrichment of substantially more than 50%, such as at least 65% or at least at least 75%, preferably at least 80%, more preferably at least 85%, such as at least 90% plants or plant parts having dwarfism-causing genes or mutations. “Dwarfism” as used herein may manifest as a reduced plant height, but may also manifest in smaller seed or grain size.

A reduced (average and/or maximum) size as used herein, may mean for instance a reduced (average and/or maximum) plant height (i.e. compared to (average and/or maximum) size or plant height of plants not having dwarfism). The skilled person will understand that size is determined and compared in a comparable context, such as at comparable developmental stage, e.g. mature plants or seeds. For seeds or grains, a reduced (average and/or maximum) size may for instance mean a reduced (average and/or maximum) length and/or a reduced (average and/or maximum) width and/or reduced (average and/or maximum) thickness, and/or circumference, and/or diameter, and/or volume, and/or weight, etc. Seed length may be determined as the maximum seed length of a seed under measurement, usually the longest axis of the seed. Seed width may be determined as the maximum seed width of a seed under measurement, usually the second longest axis, perpendicular or nearly so to the length axis. Seed thickness may be determined as the maximum seed thickness under measurement, usually the third longest axis, if needed. Seed sorting based on size may for instance be performed by sieving. A reduced (average and/or maximum) seed or grain size generally results in a reduced (average and/or maximum) seed or grain weight. Accordingly, a reduced (average and/or maximum) seed or grain size may advantageously be expressed as seeds or grains having a reduced 1000 kernel weight (e.g. expressed as the weight in gram of 1000 seeds).

In certain embodiments, dwarfism means a (average) reduction in plant height of at least 5%, preferably at least 8%, more preferably at least 10%. In certain embodiments, dwarfism means a (average) reduction in seed length of at least 5%, preferably at least 8%, more preferably at least 10%. In certain embodiments, dwarfism means a (average) reduction in 1000 kernel weight of at least 4%, preferably at least 6%, more preferably at least 8%.

“Myb80” (previously also known as Myb103) as used herein is an anther specific expressed gene. Mutants of this gene are known in other plant species and cause a male sterile phenotype. The Arabidopsis ortholog AtMyb80 (previously also known as AtMyb103) plays an important role in tapetum development, callose dissolution and exine formation in Arabidopsis thaliana anthers (Li S F, Higginson T, Parish R W: A novel MYB-related gene from Arabidopsis thaliana expressed in developing anthers. Plant Cell Physiol. 1999, 40:343-347. Higginson T, Li S F, Parish R W: AtMYB103 regulates tapetum and trichome development in Arabidopsis thaliana. Plant J. 2003, 35:177-192. Zhang Z B, Zhu J, Gao J F, Wang C, Li H, Li H, Zhang H Q, Zhang S, Wang D M, Wang Q X, Huang H, Xia H J, Yang Z N: Transcription factor AtMYB103 is required for anther development by regulating tapetum development, callose dissolution and exine formation in Arabidopsis. Plant J. 2007, 52:528-538. Phan H A, lacuone S, Li S F, Parish R W: The MYB80 transcription factor is required for pollen development and the regulation of tapetal programmed cell death in Arabidopsis thaliana. Plant Cell. 2011, 23:2209-2224. Phan H A, Li S F, Parish R W: MYB80, a regulator of tapetal and pollen development, is functionally conserved in crops. Plant Mol Biol. 2012, 78:171-183. Xu, Y., lacuone, S., Li, S. F. et al. MYB80 homologues in Arabidopsis, cotton and Brassica: regulation and functional conservation in tapetal and pollen development. BMC Plant Biol 14, 278 (2014)). Reduction of the expression of AtMyb80 in transgenic Arabidopsis plants via a co-suppression approach results in a reduced viability and fertility of the pollen produced from these plants (Higginson T, Li S F, Parish R W: AtMYB103 regulates tapetum and trichome development in Arabidopsis thaliana. Plant J. 2003, 35:177-192.). In rice an EMS based mutant was identified, where an amino acid exchange (E to K) caused a male sterile phenotype (Yan et al. (2017) Simultaneous Identification of Multiple Causal Mutations in Rice. Front. Plant Sci, doi:10.3389/fpls.2016.02055). A representative sequence of Oryza sativa (subsp. Japonica) Myb80 (OsMyb80) can be found in the Plant Transcription Factor Database as ID LOC_Os04g39470.1 (http://planttfdb.gao-lab.org/tf.php?sp=Osj&did=LOC_Os04g39470.1). A representative sequence of Arabidopsis thaliana Myb80 (AtMyb80) can be found in The Arabidopsis Information Resource (tair) as locus AT5G56110.1 (https://www.arabidopsis.org/servlets/TairObject?type=locus&name=At5g56110). In certain embodiments, the Oryza sativa orthologue of Myb80 has a coding sequence as set forth in SEQ ID NO: 1. Additional sequences are provided in Table 3. The skilled person will understand that orthologues from different species and genera can be identified based on sequence alignment, such as BLAST analysis, as described herein elsewhere.

“Dwarf11” (also called CYP724B1, D11, GNS4, or PMM1) as used herein refers to an enzyme involved in the production of brassinosteroid plant hormones. The mutant plants show a dwarf phenotype but more important also a small grain phenotype in rice. Rice plants with reduced expression of this gene show a dwarf phenotype (only approx . . . 87% of the height of a wt plant) and the average seed length is only 87% of the wt plants, whereas the seed width is not affected (Zhou, Y., Tao, Y., Zhu, J. et al. GNS4, a novel allele of DWARF11, regulates grain number and grain size in a high-yield rice variety. Rice 10, 34 (2017)). Due to this mutant phenotype, the seeds are rounder, have a different shape. This shape difference can be used to separate wt seeds from mutant seeds on specific sieves. Additionally, there are weight differences describe. The thousand kernel weight of the mutant seeds is 9.4% lower. Also this weight difference can be used in a seed purification process to separate wt seeds from mutant seeds. Due to the fact that the gene responsible for the phenotype is known, specific molecular markers can be developed for the detection of the specific mutations in the plants. Therefore, pure population of mutant seeds can be generated to calibrate the seed purification process. In certain embodiments, the Oryza sativa orthologue of Dwarf11 has a coding sequence as set forth in SEQ ID NO: 4. Additional sequences are provided in Table 3. The skilled person will understand that orthologues from different species and genera can be identified based on sequence alignment, such as BLAST analysis, as described herein elsewhere.

In a preferred embodiment, the zygosity of the gene pair according to the invention, such as the Myb80 gene and the Dwarf11 gene, is the same, i.e. both comprise a homozygous mutation or both comprise a heterozygous mutation, or alternatively both are wild type (i.e. do not comprise a mutation of the invention).

In an aspect, the invention relates to a method for generating or modifying a plant or plant part, comprising
a) providing a plant or plant part not expressing or having reduced expression, activity, and/or stability of the Myb80 gene product;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a mutation in the Myb80 gene or regulatory sequence thereof;
b) reducing or eliminating expression, activity, and/or stability of the Dwarf11 gene product in said plant or plant part; and/or introducing a mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a mutation in the Myb80 gene or regulatory sequence thereof;
b) reducing or eliminating expression, activity, and/or stability of the Dwarf11 gene product in said plant or plant part and or by introducing a mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof;
b) introducing a mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof, thereby reducing or eliminating the expression, activity, and/or stability of the Myb80 gene product;
b) introducing a mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a mutation in the Myb80 gene or regulatory sequence thereof;
b) reducing or eliminating expression, activity, and/or stability of the Dwarf11 gene product in said plant or plant part; and/or introducing a homozygous mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a mutation in the Myb80 gene or regulatory sequence thereof;
b) reducing or eliminating expression, activity, and/or stability of the Dwarf11 gene product in said plant or plant part and or by introducing a homozygous mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof;
b) introducing a homozygous mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof, thereby reducing or eliminating the expression, activity, and/or stability of the Myb80 gene product;
b) introducing a homozygous mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a homozygous mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a mutation in the Myb80 gene or regulatory sequence thereof;
b) reducing or eliminating expression, activity, and/or stability of the Dwarf11 gene product in said plant or plant part; and/or introducing a heterozygous mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a mutation in the Myb80 gene or regulatory sequence thereof;
b) reducing or eliminating expression, activity, and/or stability of the Dwarf11 gene product in said plant or plant part and or by introducing a heterozygous mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof;
b) introducing a heterozygous mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof, thereby reducing or eliminating the expression, activity, and/or stability of the Myb80 gene product;
b) introducing a heterozygous mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a heterozygous mutation in the Dwarf11 gene or regulatory sequence thereof.

In an aspect, the invention relates to a method for generating or modifying a plant or plant part, comprising
a) providing a plant or plant part having a mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a homozygous mutation in the Myb80 gene or regulatory sequence thereof;
b) reducing or eliminating expression, activity, and/or stability of the Dwarf11 gene product in said plant or plant part; and/or introducing a homozygous mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a homozygous mutation in the Myb80 gene or regulatory sequence thereof;
b) reducing or eliminating expression, activity, and/or stability of the Dwarf11 gene product in said plant or plant part and or by introducing a homozygous mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof;
b) introducing a homozygous mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof, thereby reducing or eliminating the expression, activity, and/or stability of the Myb80 gene product;
b) introducing a homozygous mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a homozygous mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a homozygous mutation in the Myb80 gene or regulatory sequence thereof;
b) reducing or eliminating expression, activity, and/or stability of the Dwarf11 gene product in said plant or plant part; and/or introducing a heterozygous mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a homozygous mutation in the Myb80 gene or regulatory sequence thereof;
b) reducing or eliminating expression, activity, and/or stability of the Dwarf11 gene product in said plant or plant part and or by introducing a heterozygous mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof;
b) introducing a heterozygous mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof, thereby reducing or eliminating the expression, activity, and/or stability of the Myb80 gene product;
b) introducing a heterozygous mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a heterozygous mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a homozygous mutation in the Myb80 gene or regulatory sequence thereof;
b) reducing or eliminating expression, activity, and/or stability of the Dwarf11 gene product in said plant or plant part; and/or introducing a mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a homozygous mutation in the Myb80 gene or regulatory sequence thereof;
b) reducing or eliminating expression, activity, and/or stability of the Dwarf11 gene product in said plant or plant part and or by introducing a mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof;
b) introducing a mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof, thereby reducing or eliminating the expression, activity, and/or stability of the Myb80 gene product;
b) introducing a mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a mutation in the Dwarf11 gene or regulatory sequence thereof.

In an aspect, the invention relates to a method for generating or modifying a plant or plant part, comprising
a) providing a plant or plant part having a homozygous mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a heterozygous mutation in the Myb80 gene or regulatory sequence thereof;
b) reducing or eliminating expression, activity, and/or stability of the Dwarf11 gene product in said plant or plant part; and/or introducing a homozygous mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a heterozygous mutation in the Myb80 gene or regulatory sequence thereof;
b) reducing or eliminating expression, activity, and/or stability of the Dwarf11 gene product in said plant or plant part and or by introducing a homozygous mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof;
b) introducing a homozygous mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof, thereby reducing or eliminating the expression, activity, and/or stability of the Myb80 gene product;
b) introducing a homozygous mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a homozygous mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a heterozygous mutation in the Myb80 gene or regulatory sequence thereof;
b) reducing or eliminating expression, activity, and/or stability of the Dwarf11 gene product in said plant or plant part; and/or introducing a heterozygous mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a heterozygous mutation in the Myb80 gene or regulatory sequence thereof;
b) reducing or eliminating expression, activity, and/or stability of the Dwarf11 gene product in said plant or plant part and or by introducing a heterozygous mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof;
b) introducing a heterozygous mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof, thereby reducing or eliminating the expression, activity, and/or stability of the Myb80 gene product;
b) introducing a heterozygous mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a heterozygous mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Myb80 gene product; and/or having a heterozygous mutation in the Myb80 gene or regulatory sequence thereof;
b) reducing or eliminating expression, activity, and/or stability of the Dwarf11 gene product in said plant or plant part; and/or introducing a mutation in the Dwarf11 gene or regulatory sequence thereof.

In an aspect, the invention relates to a method for generating or modifying a plant or plant part, comprising
a) providing a plant or plant part not expressing or having reduced expression, activity, and/or stability of the Myb80 gene product and or by having a heterozygous mutation in the Myb80 gene or regulatory sequence thereof;
b) reducing or eliminating expression, activity, and/or stability of the Dwarf11 gene product in said plant or plant part and or by introducing a mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof;
b) introducing a mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof, thereby reducing or eliminating the expression, activity, and/or stability of the Myb80 gene product;
b) introducing a mutation in the Dwarf11 gene or regulatory sequence thereof, thereby reducing or eliminating the expression, activity, and/or stability of the Dwarf11 gene product.

In an aspect, the invention relates to a method for generating or modifying a plant or plant part, comprising
a) providing a plant or plant part not expressing or having reduced expression, activity, and/or stability of the Myb80 gene product;
b) introducing a mutation in the Dwarf11 gene or regulatory sequence thereof.

In an aspect, the invention relates to a method for generating or modifying a plant or plant part, comprising
a) providing a plant or plant part having a heterozygous mutation in the Myb80 gene or regulatory sequence thereof;
b) reducing or eliminating expression, activity, and/or stability of the Dwarf11 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a mutation in the Dwarf11 gene or regulatory sequence thereof;
b) reducing or eliminating expression, activity, and/or stability of the Myb80 gene product in said plant or plant part; and/or introducing a mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and/or by having a mutation in the Dwarf11 gene or regulatory sequence thereof;
b) reducing or eliminating expression, activity, and/or stability of the Myb80 gene product in said plant or plant part and or by introducing a mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof;
b) introducing a mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof, thereby reducing or eliminating the expression, activity, and/or stability of the Dwarf11 gene product;
b) introducing a mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a mutation in the Dwarf11 gene or regulatory sequence thereof;
b) reducing or eliminating expression, activity, and/or stability of the Myb80 gene product in said plant or plant part; and/or introducing a homozygous mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and/or by having a mutation in the Dwarf11 gene or regulatory sequence thereof;
b) reducing or eliminating expression, activity, and/or stability of the Myb80 gene product in said plant or plant part and or by introducing a homozygous mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof;
b) introducing a homozygous mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof, thereby reducing or eliminating the expression, activity, and/or stability of the Dwarf11 gene product;
b) introducing a homozygous mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a homozygous mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a mutation in the Dwarf11 gene or regulatory sequence thereof;
b) reducing or eliminating expression, activity, and/or stability of the Myb80 gene product in said plant or plant part; and/or introducing a heterozygous mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and or by having a mutation in the Dwarf11 gene or regulatory sequence thereof;
b) reducing or eliminating expression, activity, and/or stability of the Myb80 gene product in said plant or plant part and or by introducing a heterozygous mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof;
b) introducing a heterozygous mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof, thereby reducing or eliminating the expression, activity, and/or stability of the Dwarf11 gene product;
b) introducing a heterozygous mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a heterozygous mutation in the Myb80 gene or regulatory sequence thereof.

In an aspect, the invention relates to a method for generating or modifying a plant or plant part, comprising
a) providing a plant or plant part having a mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a homozygous mutation in the Dwarf11 gene or regulatory sequence thereof;
b) reducing or eliminating expression, activity, and/or stability of the Myb80 gene product in said plant or plant part; and/or introducing a homozygous mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and or by having a homozygous mutation in the Dwarf11 gene or regulatory sequence thereof;
b) reducing or eliminating expression, activity, and/or stability of the Myb80 gene product in said plant or plant part and or by introducing a homozygous mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof;
b) introducing a homozygous mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof, thereby reducing or eliminating the expression, activity, and/or stability of the Dwarf11 gene product;
b) introducing a homozygous mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a homozygous mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a homozygous mutation in the Dwarf11 gene or regulatory sequence thereof;
b) reducing or eliminating expression, activity, and/or stability of the Myb80 gene product in said plant or plant part; and/or introducing a heterozygous mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and or by having a homozygous mutation in the Dwarf11 gene or regulatory sequence thereof;
b) reducing or eliminating expression, activity, and/or stability of the Myb80 gene product in said plant or plant part and or by introducing a heterozygous mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof;
b) introducing a heterozygous mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof, thereby reducing or eliminating the expression, activity, and/or stability of the Dwarf11 gene product;
b) introducing a heterozygous mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a heterozygous mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a homozygous mutation in the Dwarf11 gene or regulatory sequence thereof;
b) reducing or eliminating expression, activity, and/or stability of the Myb80 gene product in said plant or plant part; and/or introducing a mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and or by having a homozygous mutation in the Dwarf11 gene or regulatory sequence thereof;
b) reducing or eliminating expression, activity, and/or stability of the Myb80 gene product in said plant or plant part and or by introducing a mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof;
b) introducing a mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof, thereby reducing or eliminating the expression, activity, and/or stability of the Dwarf11 gene product;
b) introducing a mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a mutation in the Myb80 gene or regulatory sequence thereof.

In an aspect, the invention relates to a method for generating or modifying a plant or plant part, comprising
a) providing a plant or plant part having a homozygous mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a heterozygous mutation in the Dwarf11 gene or regulatory sequence thereof;
b) reducing or eliminating expression, activity, and/or stability of the Myb80 gene product in said plant or plant part; and/or introducing a homozygous mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and or by having a heterozygous mutation in the Dwarf11 gene or regulatory sequence thereof;
b) reducing or eliminating expression, activity, and/or stability of the Myb80 gene product in said plant or plant part and or by introducing a homozygous mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof;
b) introducing a homozygous mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof, thereby reducing or eliminating the expression, activity, and/or stability of the Dwarf11 gene product;
b) introducing a homozygous mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a homozygous mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a heterozygous mutation in the Dwarf11 gene or regulatory sequence thereof;
b) reducing or eliminating expression, activity, and/or stability of the Myb80 gene product in said plant or plant part; and/or introducing a heterozygous mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and or by having a heterozygous mutation in the Dwarf11 gene or regulatory sequence thereof;
b) reducing or eliminating expression, activity, and/or stability of the Myb80 gene product in said plant or plant part and or by introducing a heterozygous mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof;
b) introducing a heterozygous mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof, thereby reducing or eliminating the expression, activity, and/or stability of the Dwarf11 gene product;
b) introducing a heterozygous mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a heterozygous mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part not expressing or having reduced expression, activity, and/or stability of the Dwarf11 gene product; and/or having a heterozygous mutation in the Dwarf11 gene or regulatory sequence thereof;
b) reducing or eliminating expression, activity, and/or stability of the Myb80 gene product in said plant or plant part; and/or introducing a mutation in the Myb80 gene or regulatory sequence thereof.

In an aspect, the invention relates to a method for generating or modifying a plant or plant part, comprising
a) providing a plant or plant part not expressing or having reduced expression, activity, and/or stability of the Dwarf11 gene product and or by having a heterozygous mutation in the Dwarf11 gene or regulatory sequence thereof;
b) reducing or eliminating expression, activity, and/or stability of the Myb80 gene product in said plant or plant part and or by introducing a mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof;
b) introducing a mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof, thereby reducing or eliminating the expression, activity, and/or stability of the Dwarf11 gene product;
b) introducing a mutation in the Myb80 gene or regulatory sequence thereof, thereby reducing or eliminating the expression, activity, and/or stability of the Myb80 gene product.

In an aspect, the invention relates to a method for generating or modifying a plant or plant part, comprising
a) providing a plant or plant part not expressing or having reduced expression, activity, and/or stability of the Dwarf11 gene product;
b) introducing a mutation in the Myb80 gene or regulatory sequence thereof.

In an aspect, the invention relates to a method for generating or modifying a plant or plant part, comprising
a) providing a plant or plant part having a heterozygous mutation in the Dwarf11 gene or regulatory sequence thereof;
b) reducing or eliminating expression, activity, and/or stability of the Myb80 gene product in said plant or plant part.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene and the Dwarf11 gene or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene and the Dwarf11 gene or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene and the Dwarf11 gene or regulatory sequences thereof.

In an aspect, the invention relates to a method for generating or modifying a plant or plant part, comprising
a) providing a plant or plant part;
b) simultaneously or sequentially in either order mutating the Myb80 gene and the Dwarf11 gene or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene homozygously and the Dwarf11 gene or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene homozygously and the Dwarf11 gene or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene homozygously and the Dwarf11 gene or regulatory sequences thereof.

In an aspect, the invention relates to a method for generating or modifying a plant or plant part, comprising
a) providing a plant or plant part;
b simultaneously or sequentially in either order mutating the Myb80 gene homozygously and the Dwarf11 gene or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene homozygously and the Dwarf11 gene heterozygously or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene homozygously and the Dwarf11 gene heterozygously or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene homozygously and the Dwarf11 gene heterozygously or regulatory sequences thereof.

In an aspect, the invention relates to a method for generating or modifying a plant or plant part, comprising
a) providing a plant or plant part;
b) simultaneously or sequentially in either order mutating the Myb80 gene homozygously and the Dwarf11 gene heterozygously or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene homozygously and the Dwarf11 gene homozygously or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene homozygously and the Dwarf11 gene homozygously or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene homozygously and the Dwarf11 gene homozygously or regulatory sequences thereof.

In an aspect, the invention relates to a method for generating or modifying a plant or plant part, comprising
a) providing a plant or plant part;
b) simultaneously or sequentially in either order mutating the Myb80 gene homozygously and the Dwarf11 gene homozygously or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene heterozygously and the Dwarf11 gene or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene heterozygously and the Dwarf11 gene or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene heterozygously and the Dwarf11 gene or regulatory sequences thereof.

In an aspect, the invention relates to a method for generating or modifying a plant or plant part, comprising
a) providing a plant or plant part;
b) simultaneously or sequentially in either order mutating the Myb80 gene heterozygously and the Dwarf11 gene or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene heterozygously and the Dwarf11 gene homozygously or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene heterozygously and the Dwarf11 gene homozygously or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene heterozygously and the Dwarf11 gene homozygously or regulatory sequences thereof.

In an aspect, the invention relates to a method for generating or modifying a plant or plant part, comprising
a) providing a plant or plant part;
b) simultaneously or sequentially in either order mutating the Myb80 gene heterozygously and the Dwarf11 gene homozygously or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene heterozygously and the Dwarf11 gene heterozygously or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene heterozygously and the Dwarf11 gene heterozygously or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene heterozygously and the Dwarf11 gene heterozygously or regulatory sequences thereof.

In an aspect, the invention relates to a method for generating or modifying a plant or plant part, comprising
a) providing a plant or plant part;
b) simultaneously or sequentially in either order mutating the Myb80 gene heterozygously and the Dwarf11 gene heterozygously or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene and the Dwarf11 gene heterozygously or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene and the Dwarf11 gene heterozygously or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene and the Dwarf11 gene heterozygously or regulatory sequences thereof.

In an aspect, the invention relates to a method for generating or modifying a plant or plant part, comprising
a) providing a plant or plant part;
b) simultaneously or sequentially in either order mutating the Myb80 gene and the Dwarf11 gene heterozygously or regulatory sequences thereof, thereby reducing or eliminating the expression, activity, and/or stability of the Myb80 and Dwarf11 gene products.

In an aspect, the invention relates to a method for generating or modifying a plant or plant part, comprising
a) providing a plant or plant part;
b) simultaneously or sequentially in either order b1) reducing or eliminating expression, activity, and/or stability of the MYB80 gene product and the DWARF11 gene product in said plant or plant part; and/or b2) mutating the Myb80 gene and the Dwarf11 gene homozygously or regulatory sequences thereof; or a combination of b1) and b2).

In an aspect, the invention relates to a method for generating or modifying a plant or plant part, comprising
a) providing a plant or plant part;
b) simultaneously or sequentially in either order mutating the Myb80 gene and the Dwarf11 gene homozygously or regulatory sequences thereof.

In an aspect, the invention relates to a method for generating or modifying a plant or plant part, comprising
a) providing a plant or plant part;
b) simultaneously or sequentially in either order reducing or eliminating expression, activity, and/or stability of the MYB80 gene product and the DWARF11 gene product in said plant or plant part and or by mutating the Myb80 gene and the Dwarf11 gene homozygously or regulatory sequences thereof.

In an aspect, the invention relates to a method for generating or modifying a plant or plant part, comprising
a) providing a plant or plant part;
b) simultaneously or sequentially in either order mutating the Myb80 gene and the Dwarf11 gene homozygously or regulatory sequences thereof, thereby reducing or eliminating the expression, activity, and/or stability of the Myb80 and Dwarf11 gene products.

In an aspect, the invention relates to a method for generating a plant or plant part, comprising
a) crossing a first plant and a second plant; wherein said first plant and/or said second plant have a heterozygous or homozygous mutation in (both) the MYB80 gene and the DWARF11 gene on the same chromosome;
b) harvesting seeds;
c) optionally selecting seeds having a homozygous or heterozygous mutation in (both) the Myb80 gene and the Dwarf11 gene;
d) optionally sowing said seeds.

In an aspect, the invention relates to a method for generating a plant or plant part, comprising
a) crossing a first plant and a second plant; wherein said first plant and said second plant have a heterozygous mutation in (both) the MYB80 gene and the DWARF11 gene on the same chromosome;
b) harvesting seeds;
c) optionally selecting seeds having a homozygous or heterozygous mutation in (both) the Myb80 gene and the Dwarf11 gene;
d) optionally sowing said seeds.

In an aspect, the invention relates to a method for generating a plant or plant part, comprising
a) crossing a first plant and a second plant; wherein only one of said first plant or said second plant has a homozygous mutation in (both) the MYB80 gene and the DWARF11 gene on the same chromosome;
b) harvesting seeds;
c) optionally selecting seeds having a homozygous or heterozygous mutation in (both) the Myb80 gene and the Dwarf11 gene;
d) optionally sowing said seeds.

In an aspect, the invention relates to a method for generating a plant or plant part, comprising
a) crossing a first plant and a second plant; wherein said first plant has a homozygous mutation in (both) the MYB80 gene and the DWARF11 gene on the same chromosome, and wherein said second plant has a heterozygous mutation in (both) the MYB80 gene and the DWARF11 gene on the same chromosome;
b) harvesting seeds;
c) optionally selecting seeds having a homozygous or heterozygous mutation in (both) the Myb80 gene and the Dwarf11 gene;
d) optionally sowing said seeds.

In an aspect, the invention relates to a method for generating a plant or plant part, comprising
a) crossing a first plant and a second plant; wherein said first plant and/or said second plant have a heterozygous or homozygous mutation in (both) the MYB80 gene and the DWARF11 gene on the same chromosome, thereby lacking or having reduced expression, activity, and/or stability of the Myb80 and Dwarf11 gene products;
b) harvesting seeds;
c) optionally selecting seeds having a homozygous or heterozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for generating a plant or plant part, comprising
a) crossing a first plant and a second plant; wherein said first plant and said second plant have a heterozygous mutation in (both) the MYB80 gene and the DWARF11 gene on the same chromosome, thereby lacking or having reduced expression, activity, and/or stability of the Myb80 and Dwarf11 gene products;
b) harvesting seeds;
c) optionally selecting seeds having a homozygous or heterozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for generating a plant or plant part, comprising
a) crossing a first plant and a second plant; wherein only one of said first plant or said second plant has a homozygous mutation in (both) the MYB80 gene and the DWARF11 gene on the same chromosome, thereby lacking or having reduced expression, activity, and/or stability of the Myb80 and Dwarf11 gene products;
b) harvesting seeds;
c) optionally selecting seeds having a homozygous or heterozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for generating a plant or plant part, comprising
a) crossing a first plant and a second plant; wherein said first plant has a homozygous mutation in (both) the MYB80 gene and the DWARF11 gene on the same chromosome, and wherein said second plant has a heterozygous mutation in (both) the MYB80 gene and the DWARF11 gene on the same chromosome, thereby lacking or having reduced expression, activity, and/or stability of the Myb80 and Dwarf11 gene products;
b) harvesting seeds;
c) optionally selecting seeds having a homozygous or heterozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for generating a plant or plant part, comprising
a) crossing a first plant and a second plant; wherein said first plant and/or said second plant lack or have reduced expression, activity, and/or stability of (both) the MYB80 gene product and the DWARF11 gene product;
b) harvesting seeds;
c) optionally selecting seeds lacking or having reduced expression, activity, and/or stability of (both) the MYB80 gene product and the DWARF11 gene product;
d) optionally sowing said seeds.

In an aspect, the invention relates to a method for generating a plant or plant part, comprising
a) crossing a first plant and a second plant; wherein said first plant and/or said second plant lack or have reduced expression, activity, and/or stability of (both) the MYB80 gene product and the DWARF11 gene product by having a homozygous or heterozygous mutation in (both) the Myb80 gene and the Dwarf11 gene.;
b) harvesting seeds;
c) optionally selecting seeds lacking or having reduced expression, activity, and/or stability of (both) the MYB80 gene product and the DWARF11 gene product and/or having a homozygous or heterozygous mutation in (both) the Myb80 gene and the Dwarf11 gene;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant and/or said second plant have a heterozygous or homozygous mutation in (both) the MYB80 gene and the DWARF11 gene on the same chromosome;
b harvesting seeds;
c) selecting seeds having a homozygous mutation in (both) the Myb80 gene and the Dwarf11 gene;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant and said second plant have a heterozygous mutation in (both) the MYB80 gene and the DWARF11 gene on the same chromosome;
b) harvesting seeds;
c) selecting seeds having a homozygous mutation in (both) the Myb80 gene and the Dwarf11 gene;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein only one of said first plant or said second plant has a homozygous mutation in (both) the MYB80 gene and the DWARF11 gene on the same chromosome;
b) harvesting seeds;
c) selecting seeds having a homozygous mutation in (both) the Myb80 gene and the Dwarf11 gene;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant has a homozygous mutation in (both) the MYB80 gene and the DWARF11 gene on the same chromosome, and wherein said second plant has a heterozygous mutation in (both) the MYB80 gene and the DWARF11 gene on the same chromosome;
b) harvesting seeds;
c) selecting seeds having a homozygous mutation in (both) the Myb80 gene and the Dwarf11 gene;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant and/or said second plant have a heterozygous or homozygous mutation in (both) the MYB80 gene and the DWARF11 gene on the same chromosome, thereby lacking or having reduced expression, activity, and/or stability of the Myb80 and Dwarf11 gene products;
b) harvesting seeds;
c) selecting seeds having a homozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant and said second plant have a heterozygous mutation in (both) the MYB80 gene and the DWARF11 gene on the same chromosome, thereby lacking or having reduced expression, activity, and/or stability of the Myb80 and Dwarf11 gene products;
b) harvesting seeds;
c) selecting seeds having a homozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein only one of said first plant or said second plant has a homozygous mutation in (both) the MYB80 gene and the DWARF11 gene on the same chromosome, thereby lacking or having reduced expression, activity, and/or stability of the Myb80 and Dwarf11 gene products;
b harvesting seeds;
c) selecting seeds having a homozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant has a homozygous mutation in (both) the MYB80 gene and the DWARF11 gene on the same chromosome, and wherein said second plant has a heterozygous mutation in (both) the MYB80 gene and the DWARF11 gene on the same chromosome, thereby lacking or having reduced expression, activity, and/or stability of the Myb80 and Dwarf11 gene products;
b) harvesting seeds;
c) selecting seeds having a homozygous mutation in (both) the Myb80 gene and the Dwarf11 gene and/or lacking or having reduced expression, activity, and/or stability of the Myb80 and Dwarf11 gene products;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant and/or said second plant lack or have reduced expression, activity, and/or stability of (both) the MYB80 gene product and the DWARF11 gene product;
b) harvesting seeds;
c) selecting seeds lacking or having reduced expression, activity, and/or stability of (both) the MYB80 gene product and the DWARF11 gene product;
d) optionally sowing said seeds.

In an aspect, the invention relates to a method for producing, obtaining, or selecting a plant or plant part, preferably a seed, comprising
a) providing a (bulk or unselected) mixture of plants or plant parts resulting from a cross between a first plant and a second plant; wherein said first plant and/or said second plant lack or have reduced expression, activity, and/or stability of (both) the MYB80 gene product and the DWARF11 gene product by having a homozygous or heterozygous mutation in (both) the Myb80 gene and the Dwarf11 gene.;
b) harvesting seeds;
c) selecting seeds lacking or having reduced expression, activity, and/or stability of (both) the MYB80 gene product and the DWARF11 gene product and/or having a homozygous mutation in (both) the Myb80 gene and the Dwarf11 gene;
d) optionally sowing said seeds.

In an aspect, the invention relates to a method for identifying and/or selecting a plant or plant part, comprising screening for the presence of a homozygous mutation in the DWARF11 gene in (the genome of) a plant or plant part resulting from a cross between a first plant and a second plant; wherein said first plant and/or said second plant have a heterozygous mutation in the MYB80 gene and the DWARF11 gene on the same chromosome; and optionally selecting a plant or plant part comprising a homozygous mutation in the DWARF11 gene.

In an aspect, the invention relates to a method for identifying and/or selecting a plant or plant part, comprising screening for the presence of a homozygous mutation in the DWARF11 gene in (the genome of) a plant or plant part resulting from a cross between a first plant and a second plant; wherein said first plant and said second plant have a heterozygous mutation in the MYB80 gene and the DWARF11 gene on the same chromosome; and optionally selecting a plant or plant part comprising a homozygous mutation in the DWARF11 gene.

In an aspect, the invention relates to a method for identifying and/or selecting a plant or plant part, comprising screening for the presence of a homozygous mutation in the DWARF11 gene in (the genome of) a plant or plant part resulting from a cross between a first plant and a second plant; wherein said first plant has a homozygous mutation in the MYB80 gene and the DWARF11 gene on the same chromosome and wherein said second plant has a heterozygous mutation in the MYB80 gene and the DWARF11 gene on the same chromosome; and optionally selecting a plant or plant part comprising a homozygous mutation in the DWARF11 gene.

In an aspect, the invention relates to a method for identifying and/or selecting a plant or plant part, comprising screening for the presence of a (homozygous or heterozygous) mutation in the DWARF11 gene in (the genome of) a plant or plant part; and optionally selecting a plant or plant part comprising a (homozygous or heterozygous) mutation in the DWARF11 gene.

In an aspect, the invention relates to a method for identifying and/or selecting a plant or plant part, comprising screening for the presence of a (homozygous or heterozygous) mutation in the Myb80 gene in (the genome of) a plant or plant part; and optionally selecting a plant or plant part comprising a (homozygous or heterozygous) mutation in the Myb80 gene.

In an aspect, the invention relates to a method for identifying and/or selecting a plant or plant part, comprising screening for the presence of a (homozygous or heterozygous) mutation in the DWARF11 gene in (the genome of) a plant or plant part having a (homozygous or heterozygous) mutation in the Myb80 gene; and optionally selecting a plant or plant part comprising a (homozygous or heterozygous) mutation in the DWARF11 gene.

In an aspect, the invention relates to a method for identifying and/or selecting a plant or plant part, comprising screening for the presence of a (homozygous or heterozygous) mutation in the Myb80 gene in (the genome of) a plant or plant part having a (homozygous or heterozygous) mutation in the Dwarf11 gene; and optionally selecting a plant or plant part comprising a (homozygous or heterozygous) mutation in the Myb80 gene.

In an aspect, the invention relates to a method for identifying and/or selecting a plant or plant part, comprising screening for the presence of a (homozygous or heterozygous) mutation in the DWARF11 gene in (the genome of) a plant or plant part resulting from a cross between a first plant and a second plant; wherein said first plant and/or said second plant have a heterozygous mutation in the MYB80 gene and the DWARF11 gene on the same chromosome; and optionally selecting a plant or plant part comprising a (homozygous or heterozygous) mutation in the DWARF11 gene.

In an aspect, the invention relates to a method for identifying and/or selecting a plant or plant part, comprising screening for the presence of a (homozygous or heterozygous) mutation in the Myb80 gene in (the genome of) a plant or plant part resulting from a cross between a first plant and a second plant; wherein said first plant and/or said second plant have a heterozygous mutation in the MYB80 gene and the DWARF11 gene on the same chromosome; and optionally selecting a plant or plant part comprising a (homozygous or heterozygous) mutation in the Myb80 gene.

In an aspect, the invention relates to a method for identifying and/or selecting a plant or plant part, comprising screening for the presence of a (homozygous or heterozygous) mutation in the DWARF11 gene in (the genome of) a plant or plant part having a (homozygous or heterozygous) mutation in the Myb80 gene and resulting from a cross between a first plant and a second plant; wherein said first plant and/or said second plant have a heterozygous mutation in the MYB80 gene and the DWARF11 gene on the same chromosome; and optionally selecting a plant or plant part comprising a (homozygous or heterozygous) mutation in the DWARF11 gene.

In an aspect, the invention relates to a method for identifying and/or selecting a plant or plant part, comprising screening for the presence of a (homozygous or heterozygous) mutation in the Myb80 gene in (the genome of) a plant or plant part having a (homozygous or heterozygous) mutation in the Dwarf11 gene and resulting from a cross between a first plant and a second plant; wherein said first plant and/or said second plant have a heterozygous mutation in the MYB80 gene and the DWARF11 gene on the same chromosome; and optionally selecting a plant or plant part comprising a (homozygous or heterozygous) mutation in the Myb80 gene.

In an aspect, the invention relates to a method for identifying and/or selecting a plant or plant part, comprising screening for the presence of a (homozygous or heterozygous) mutation in the DWARF11 gene in (the genome of) a plant or plant part resulting from a cross between a first plant and a second plant; wherein said first plant has a homozygous mutation in the MYB80 gene and the DWARF11 gene on the same chromosome and said second plant has a heterozygous mutation in the MYB80 gene and the DWARF11 gene on the same chromosome; and optionally selecting a plant or plant part comprising a (homozygous or heterozygous) mutation in the DWARF11 gene.

In an aspect, the invention relates to a method for identifying and/or selecting a plant or plant part, comprising screening for the presence of a (homozygous or heterozygous) mutation in the Myb80 gene in (the genome of) a plant or plant part resulting from a cross between a first plant and a second plant; wherein said first plant has a homozygous mutation in the MYB80 gene and the DWARF11 gene on the same chromosome and said second plant has a heterozygous mutation in the MYB80 gene and the DWARF11 gene on the same chromosome; and optionally selecting a plant or plant part comprising a (homozygous or heterozygous) mutation in the Myb80 gene.

In an aspect, the invention relates to a method for identifying and/or selecting a plant or plant part, comprising screening for the presence of a (homozygous or heterozygous) mutation in the DWARF11 gene in (the genome of) a plant or plant part having a mutation in the Myb80 gene and resulting from a cross between a first plant and a second plant; wherein said first plant has a homozygous mutation in the MYB80 gene and the DWARF11 gene on the same chromosome and said second plant has a heterozygous mutation in the MYB80 gene and the DWARF11 gene on the same chromosome; and optionally selecting a plant or plant part comprising a (homozygous or heterozygous) mutation in the DWARF11 gene.

In an aspect, the invention relates to a method for identifying and/or selecting a plant or plant part, comprising screening for the presence of a (homozygous or heterozygous) mutation in the Myb80 gene in (the genome of) a plant or plant part having a mutation in the Dwarf11 gene and resulting from a cross between a first plant and a second plant; wherein said first plant has a homozygous mutation in the MYB80 gene and the DWARF11 gene on the same chromosome and said second plant has a heterozygous mutation in the MYB80 gene and the DWARF11 gene on the same chromosome; and optionally selecting a plant or plant part comprising a (homozygous or heterozygous) mutation in the Myb80 gene.

In certain embodiments, in particular in embodiments relating to the production of plants or seeds resulting in plants having genetic male sterility, plants or plant parts, such as seeds, are selected which have a homozygous Dwarf11 mutation. Such seeds may be selected based on molecular markers, but may advantageously also be selected phenotypically, for instance based on seed size, as described herein elsewhere. Accordingly, in certain embodiments, the methods according to the invention as described herein are methods for producing male sterile plants or plant parts, such as seeds, preferably methods for producing genetic male sterile plants or plant parts, such as seeds.

Such plants or plant parts, such as seeds may then be used in hybrid crosses or hybrid breeding, in order to generate hybrid seeds, as such plants due to their male sterility cannot be self-pollinated but can only be cross-pollinated by a pollen donor, which results in hybrid seeds. Hybrid breeding typically results in uniform (hybrid) seed lots. Accordingly, no specific seed selection is required, and the entire harvested lot is useable. Accordingly, in certain embodiments, the methods according to the invention as described herein are methods for producing hybrid plants or plant parts, such as hybrid seeds.

In an aspect, the invention relates to a method for producing (hybrid) plants or plant parts, comprising sowing seeds resulting from or harvested from a cross between a first plant and a second plant, wherein (only) said first or second plant comprises a homozygous mutation in the Myb80 gene and the Dwarf11 gene on the same chromosome.

In an aspect, the invention relates to a method for producing (hybrid) plants or plant parts, comprising crossing a first plant and a second plant, wherein (only) said first or second plant comprises a homozygous mutation in the Myb80 gene and the Dwarf11 gene on the same chromosome, and harvesting seeds; optionally further comprising sowing said harvested seeds.

In an aspect, the invention relates to a method for producing (hybrid) plants or plant parts, comprising sowing seeds resulting from or harvested from a cross between a first plant and a second plant, wherein (only) said first or second plant comprises a homozygous mutation in the Myb80 gene and the Dwarf11 gene on the same chromosome, thereby lacking or having reduced expression, activity, and/or stability of the Myb80 and Dwarf11 gene products.

In an aspect, the invention relates to a method for producing (hybrid) plants or plant parts, comprising crossing a first plant and a second plant, wherein (only) said first or second plant comprises a homozygous mutation in the Myb80 gene and the Dwarf11 gene on the same chromosome, and harvesting seeds; optionally further comprising sowing said harvested seeds, thereby lacking or having reduced expression, activity, and/or stability of the Myb80 and Dwarf11 gene products.

In an aspect, the invention relates to a method for producing (hybrid) plants or plant parts, comprising sowing seeds resulting from or harvested from a cross between a first plant and a second plant, wherein (only) said first or second plant lacks or has reduced expression, activity, and/or stability of the Myb80 and Dwarf11 gene products.

In an aspect, the invention relates to a method for producing (hybrid) plants or plant parts, comprising crossing a first plant and a second plant, wherein (only) said first or second plant lacks or has reduced expression, activity, and/or stability of the Myb80 and Dwarf11 gene products, and harvesting seeds; optionally further comprising sowing said harvested seeds.

In an aspect, the invention relates to a method for producing (hybrid) plants or plant parts, comprising sowing seeds resulting from or harvested from a cross between a first plant and a second plant, wherein (only) said first or second plant lacks or has reduced expression, activity, and/or stability of the Myb80 and Dwarf11 gene products and or by having a homozygous mutation in the Myb80 gene and the Dwarf11 gene on the same chromosome.

In an aspect, the invention relates to a method for producing (hybrid) plants or plant parts, comprising crossing a first plant and a second plant, wherein (only) said first or second plant lacks or has reduced expression, activity, and/or stability of the Myb80 and Dwarf11 gene products and or by having a homozygous mutation in the Myb80 gene and the Dwarf11 gene on the same chromosome, and harvesting seeds; optionally further comprising sowing said harvested seeds.

In an aspect, the invention relates to a method for producing (hybrid) plants or plant parts, comprising crossing a first plant and a second plant, wherein (only) said first or second plant is a plant according to the invention as described herein, or is a plant generated, modified, identified, or selected according to the methods of the invention as described herein.

In an aspect, the invention relates to a method for producing (genetic male sterile) plants or plant parts, comprising crossing a first plant and a second plant, wherein (only) said first or second plant is a plant according to the invention as described herein, or is a plant generated, modified, identified, or selected according to the methods of the invention as described herein.

In an aspect, the invention relates to a method for producing (genetic male sterile dwarf) plants or plant parts, comprising crossing a first plant and a second plant, wherein (only) said first or second plant is a plant according to the invention as described herein, or is a plant generated, modified, identified, or selected according to the methods of the invention as described herein.

In an aspect, the invention relates to a (hybrid) plant or plant part generated, modified, identified, or selected according to the methods for generating, modifying, identifying, or selecting according to the invention as described herein, or (first generation) offspring thereof.

In an aspect, the invention relates to a (genetic male sterile) plant or plant part generated, modified, identified, or selected according to the methods for generating, modifying, identifying, or selecting according to the invention as described herein, or (first generation) offspring thereof.

In an aspect, the invention relates to a (genetic male sterile dwarf) plant or plant part generated, modified, identified, or selected according to the methods for generating, modifying, identifying, or selecting according to the invention as described herein, or (first generation) offspring thereof.

In an aspect, the invention relates to a plants or plant part, comprising a homozygous mutation in the Myb80 gene and the Dwarf11 gene on the same chromosome, or (first generation) offspring thereof.

In an aspect, the invention relates to a plants or plant part, comprising a heterozygous mutation in the Myb80 gene and the Dwarf11 gene on the same chromosome, or (first generation) offspring thereof.

In an aspect, the invention relates to a plants or plant part, lacking or having reduced expression, activity, and/or stability of a Myb80 gene and a Dwarf11 gene, or (first generation) offspring thereof.

In an aspect, the invention relates to a plants or plant part, comprising a homozygous mutation in the Myb80 gene and the Dwarf11 gene on the same chromosome and or thereby lacking or having reduced expression, activity, and/or stability of a Myb80 gene and a Dwarf11 gene, or (first generation) offspring thereof.

In an aspect, the invention relates to a plants or plant part, comprising a heterozygous mutation in the Myb80 gene and the Dwarf11 gene on the same chromosome and or thereby lacking or having reduced expression, activity, and/or stability of a Myb80 gene and a Dwarf11 gene, or (first generation) offspring thereof.

In an aspect, the invention relates to a plants or plant part, lacking or having reduced expression, activity, and/or stability of a Myb80 gene and a Dwarf11 gene and or by having a homozygous mutation in the Myb80 gene and the Dwarf11 gene on the same chromosome, or (first generation) offspring thereof.

In an aspect, the invention relates to a plants or plant part, lacking or having reduced expression, activity, and/or stability of a Myb80 gene and a Dwarf11 gene and or by having a heterozygous mutation in the Myb80 gene and the Dwarf11 gene on the same chromosome, or (first generation) offspring thereof.

In certain embodiments, the methods according to the invention as described herein are methods for generating, producing, obtaining, identifying, or selecting plants or plant parts which are male or female sterile, preferably male sterile. In certain embodiments, the methods according to the invention as described herein are methods for generating, producing, obtaining, identifying, or selecting plants or plant parts which are genetic male or genetic female sterile, preferably genetic male sterile.

In certain embodiments, the methods according to the invention as described herein are methods for generating, producing, obtaining, identifying, or selecting plants or plant parts carrying a (homozygous or heterozygous) male or female sterility gene (allele), preferably a (homozygous or heterozygous) male sterility gene (allele). In certain embodiments, the methods according to the invention as described herein are methods for generating, producing, obtaining, identifying, or selecting plants or plant parts carrying a (homozygous or heterozygous) genetic male or genetic female sterility gene (allele), preferably a (homozygous or heterozygous) genetic male sterility gene (allele).

In certain embodiments, the methods according to the invention as described herein are methods for generating, producing, obtaining, identifying, or selecting plants or plant parts which have a (homozygous or heterozygous) Myb80 (knockout) mutation, such as a (homozygous or heterozygous) Myb80 (knockout) mutation causing or capable of causing (e.g. when homozygous) genetic male sterility.

In certain embodiments, the methods according to the invention as described herein are methods for generating, producing, obtaining, identifying, or selecting plants or plant parts in which the Myb80 gene is (homozygously or heterozygously) knocked out or deleted.

In certain embodiments, the methods according to the invention as described herein are methods for generating, producing, obtaining, identifying, or selecting plants or plant parts exhibiting dwarfism, preferably seed dwarfism.

In certain embodiments, the methods according to the invention as described herein are methods for generating, producing, obtaining, identifying, or selecting plants or plant parts carrying a (homozygous or heterozygous) dwarfism gene (allele), preferably a (homozygous or heterozygous) seed dwarfism gene (allele).

In certain embodiments, the methods according to the invention as described herein are methods for generating, producing, obtaining, identifying, or selecting plants or plant parts which have a (homozygous or heterozygous) Dwarf11 (knockout) mutation, such as a (homozygous or heterozygous) Dwarf11 (knockout) mutation causing or capable of causing (e.g. when homozygous) dwarfism, such as seed dwarfism.

In certain embodiments, the methods according to the invention as described herein are methods for generating, producing, obtaining, identifying, or selecting plants or plant parts in which the Dwarf11 gene is (homozygously or heterozygously) knocked out or deleted.

In certain embodiments, the methods according to the invention as described herein are methods for generating, producing, obtaining, identifying, or selecting plants or plant parts which are male or female sterile, preferably male sterile, and exhibiting dwarfism, preferably seed dwarfism. In certain embodiments, the methods according to the invention as described herein are methods for generating, producing, obtaining, identifying, or selecting plants or plant parts which are genetic male or genetic female sterile, preferably genetic male sterile, and exhibiting dwarfism, preferably seed dwarfism.

In certain embodiments, the methods according to the invention as described herein are methods for generating, producing, obtaining, identifying, or selecting plants or plant parts carrying a (homozygous or heterozygous) male or female sterility gene (allele), preferably a (homozygous or heterozygous) male sterility gene (allele), and carrying a (homozygous or heterozygous) dwarfism gene (allele), preferably a (homozygous or heterozygous) seed dwarfism gene (allele). In certain embodiments, the methods according to the invention as described herein are methods for generating, producing, obtaining, identifying, or selecting plants or plant parts carrying a (homozygous or heterozygous) genetic male or genetic female sterility gene (allele), preferably a (homozygous or heterozygous) genetic male sterility gene (allele), and carrying a (homozygous or heterozygous) dwarfism gene (allele), preferably a (homozygous or heterozygous) seed dwarfism gene (allele).

In certain embodiments, the methods according to the invention as described herein are methods for generating, producing, obtaining, identifying, or selecting plants or plant parts which have a (homozygous or heterozygous) Myb80 (knockout) mutation, such as a (homozygous or heterozygous) Myb80 (knockout) mutation causing or capable of causing (e.g. when homozygous) genetic male sterility, and which have a (homozygous or heterozygous) Dwarf11 (knockout) mutation, such as a (homozygous or heterozygous) Dwarf11 (knockout) mutation causing or capable of causing (e.g. when homozygous) dwarfism, such as seed dwarfism.

In certain embodiments, the methods according to the invention as described herein are methods for generating, producing, obtaining, identifying, or selecting plants or plant parts in which the Myb80 gene is (homozygously or heterozygously) knocked out or deleted, and in which the Dwarf11 gene is (homozygously or heterozygously) knocked out or deleted.

In certain embodiments, the mutation according to the invention as described herein is a nonsense or missense mutation. In certain embodiments, the mutation according to the invention as described herein is a frameshift mutation. In certain embodiments, the mutation according to the invention as described herein is an indel mutation. In certain embodiments, the mutation according to the invention as described herein is a dominant (negative) mutation. In certain embodiments, the mutation according to the invention as described herein is a recessive mutation. In certain embodiments, the mutation according to the invention as described herein is a knock-out or knock-down mutation. Preferably, the mutation is a recessive mutation.

In certain preferred embodiments, the mutation is a knockout mutation, such as a frameshift mutation, and indel, or a nonsense mutation. In certain embodiments, the Myb80 mutation is a mutation causing (or capable of causing, e.g. when homozygous) genetic male sterility. In certain embodiments, the Dwarf11 mutation is a mutation causing (or capable of causing, e.g. when homozygous) dwarfism. In certain embodiments, the Myb80 mutation is a knockout mutation, such as a frameshift mutation, and indel, or a nonsense mutation causing (or capable of causing, e.g. when homozygous) genetic male sterility. In certain embodiments, the Dwarf11 mutation is a knockout mutation, such as a frameshift mutation, and indel, or a nonsense mutation causing (or capable of causing, e.g. when homozygous) dwarfism.

In certain embodiments, the mutation according to the invention as described herein is in the coding sequence, a splicing signal, or a regulatory element, such as a promoter or a sequence affecting the expression or function of said coding sequence, a splicing signal, or a regulatory element. In certain embodiments, the mutation according to the invention as described herein is in the first exon. As used herein, the first exon is the most 5′ exon. In certain embodiments, the mutation according to the invention as described herein is in the first coding exon. As used herein, the first coding exon is the exon containing the start codon. The skilled person will understand that the first coding exon may not necessarily be the first exon.

Mutations can be introduced by any means known in the art, for instance as described herein elsewhere (e.g. knockout or knockdown mutations by random or site-directed mutagenesis, including CRISPR/Cas, ZFN, TALEN, meganucleases, RNAi, TILLING, etc.). In certain embodiments, the mutation according to the invention as described herein is introduced by mutagenesis. In certain embodiments, the mutation according to the invention as described herein is introduced by random mutagenesis, preferably TILLING. In certain embodiments, the mutation according to the invention as described herein is introduced by TILLING. In certain embodiments, the mutation according to the invention as described herein is introduced by non-random mutagenesis. In certain embodiments, the mutation according to the invention as described herein is introduced by site-directed mutagenesis. As used herein, site-directed mutagenesis refers to sequence-specific (or sequence-dependent) or target-specific (or target-dependent) mutagenesis, as described herein elsewhere. In certain embodiments, the mutation according to the invention as described herein is introduced by gene-editing. In certain embodiments, the mutation according to the invention as described herein is introduced by genome-editing. In certain embodiments, the mutation according to the invention as described herein is introduced by designer nucleases In certain embodiments, the mutation according to the invention as described herein is introduced by CRISPR/Cas, zinc finger nucleases, TALEN, or meganucleases, as described herein elsewhere. In certain embodiments, the mutation according to the invention as described herein is introduced by CRISPR/Cas. By means of example, gRNAs may be designed which target the Myb80 gene (preferably the first exon) or the Dwarf11 gene (preferably the first exon). CRISPR/Cas mediated gene-editing results in the generation of indels (preferably in the first exon), typically resulting in frameshifts, and a knockout of the gene.

In certain embodiments, the plants or plant parts according to the invention as described herein, or generated, produced, obtained, identified, and/or selected according to the invention as described herein, are transgenic plants or plant parts.

In certain embodiments, the mutation according to the invention as described herein is knocked-in.

In certain embodiments, the plants or plant parts according to the invention as described herein, or generated, produced, obtained, identified, and/or selected according to the invention as described herein, are from a species in which the gene pair (such as the Myb80 and Dwarf11 genes) are within 1 Mbp or 1 cM from each other (i.e. on the same chromosome, preferably on the same chromosome arm). In certain embodiments, the plants or plant parts according to the invention as described herein, or generated, produced, obtained, identified, and/or selected according to the invention as described herein, are from the family of Poaceae. In certain embodiments, the plants or plant parts according to the invention as described herein, or generated, produced, obtained, identified, and/or selected according to the invention as described herein, are from a species of the family of Poaceae in which the gene pair (such as the Myb80 and Dwarf11 genes) are within 1 Mbp or 1 cM from each other (i.e. on the same chromosome, preferably on the same chromosome arm). In certain embodiments, the plants or plant parts according to the invention as described herein, or generated, produced, obtained, identified, and/or selected according to the invention as described herein, are from the subfamily of Pooideae, Panicoideae, Chloridoideae, Pharoideae, Bambusoideae, or Oryzoideae. In certain embodiments, the plants or plant parts according to the invention as described herein, or generated, produced, obtained, identified, and/or selected according to the invention as described herein, are from a species of the subfamily of Pooideae, Panicoideae, Chloridoideae, Pharoideae, Bambusoideae, or Oryzoideae in which the gene pair (such as the Myb80 and Dwarf11 genes) are within 1 Mbp or 1 cM from each other (i.e. on the same chromosome, preferably on the same chromosome arm). In certain embodiments, the plants or plant parts according to the invention as described herein, or generated, produced, obtained, identified, and/or selected according to the invention as described herein, are from the genus Aegilops, Brachypodium, Cenchrus, Chasmanthium, Digitaria, Eleusine, Eragrostis, Hordeum, Leersia, Lolium, Miscanthus, Oropetium, Oryza, Panicum, Paspalum, Pharus, Phyllostachys, Saccharum, Secale, Setaria, Sorghum, Thinopyrum, Triticum, Urochloa, Avena, Poa, Phleum, Festuca, or Deschampsia. In certain embodiments, the plants or plant parts according to the invention as described herein, or generated, produced, obtained, identified, and/or selected according to the invention as described herein, are from a species of the genus of Aegilops, Brachypodium, Cenchrus, Chasmanthium, Digitaria, Eleusine, Eragrostis, Hordeum, Leersia, Lolium, Miscanthus, Oropetium, Oryza, Panicum, Paspalum, Pharus, Phyllostachys, Saccharum, Secale, Setaria, Sorghum, Thinopyrum, Triticum, Urochloa, Avena, Poa, Phleum, Festuca, or Deschampsia in which the gene pair (such as the Myb80 and Dwarf11 genes) are within 1 Mbp or 1 cM from each other (i.e. on the same chromosome, preferably on the same chromosome arm). In certain embodiments, the plants or plant parts according to the invention as described herein, or generated, produced, obtained, identified, and/or selected according to the invention as described herein, are from the genus Aegilops, Brachypodium, Cenchrus, Chasmanthium, Digitaria, Eleusine, Eragrostis, Hordeum, Leersia, Lolium, Miscanthus, Oropetium, Zea, Oryza, Panicum, Paspalum, Pharus, Phyllostachys, Saccharum, Secale, Setaria, Sorghum, Thinopyrum, Triticum, Urochloa, Avena, Poa, Phleum, Festuca, or Deschampsia. In certain embodiments, the plants or plant parts according to the invention as described herein, or generated, produced, obtained, identified, and/or selected according to the invention as described herein, are from the genus Aegilops, Brachypodium, Cenchrus, Chasmanthium, Digitaria, Eleusine, Eragrostis, Hordeum, Leersia, Lolium, Miscanthus, Oropetium, Oryza, Panicum, Paspalum, Pharus, Phyllostachys, Saccharum, Secale, Setaria, Sorghum, Thinopyrum, Triticum, or Urochloa. In certain embodiments, the plants or plant parts according to the invention as described herein, or generated, produced, obtained, identified, and/or selected according to the invention as described herein, are from the genus Triticum, Sorghum, Secale, Hordeum, Oryza, Avena, Aegilops, Brachypodium, Leersia, Setaria, Zea, Poa, Phleum, Lolium, Festuca, or Deschampsia. In certain embodiments, the plants or plant parts according to the invention as described herein, or generated, produced, obtained, identified, and/or selected according to the invention as described herein, are from the genus Triticum, Sorghum, Secale, Hordeum, Oryza, Avena, Aegilops, Brachypodium, Leersia, Setaria, Poa, Phleum, Lolium, Festuca, or Deschampsia. In certain embodiments, the plants or plant parts according to the invention as described herein, or generated, produced, obtained, identified, and/or selected according to the invention as described herein, are from the genus Triticum, Sorghum, Secale, Hordeum, Oryza, Avena, Aegilops, Brachypodium, Leersia, or Setaria, In certain embodiments, the plants or plant parts according to the invention as described herein, or generated, produced, obtained, identified, and/or selected according to the invention as described herein, are from the genus Triticum, Sorghum, Hordeum, Oryza, Aegilops, Brachypodium, Leersia, or Setaria, In certain embodiments, the plants or plant parts according to the invention as described herein, or generated, produced, obtained, identified, and/or selected according to the invention as described herein, are from the species Aegilops tauschii, Brachypodium distachyon, Brachypodium mexicanum, Brachypodium stacei, Brachypodium sylvaticum, Cenchrus purpureus, Chasmanthium laxum, Digitaria exilis, Eleusine coracana, Eleusine coracana, Eragrostis curvula, Eragrostis tef, Hordeum vulgare, Leersia perrieri, Lolium perenne, Miscanthus sinensis, Oropetium thomaeum, Oryza alta, Oryza brachyantha, Oryza glaberrima, Oryza glumipatula, Oryza longistaminata, Oryza meridionalis, Oryza nivara, Oryza punctata, Oryza rufipogon, Oryza sativa Indica Group, Oryza sativa Japonica Group, Panicum hallii FIL2, Panicum hallii HAL2, Panicum virgatum, Panicum virgatum, Paspalum vaginatum, Pharus latifolius, Phyllostachys edulis, Saccharum spontaneum, Secale cereale, Setaria italica, Setaria viridis, Sorghum bicolor, Thinopyrum intermedium, Triticum aestivum, Triticum dicoccoides, Triticum spelta, Triticum turgidum, or Urochloa fusca. In certain embodiments, the plants or plant parts according to the invention as described herein, or generated, produced, obtained, identified, and/or selected according to the invention as described herein, are from the species Triticum aestivum, Aegilops tauschii, Brachypodium distachyon, Hordeum vulgare, Leersia perrieri, Oryza barthii, Oryza brachyantha, Oryza glaberrima, Oryza glumipatula, Oryza longistaminata, Oryza meridionalis, Oryza nivara, Oryza punctata, Oryza rufipogon, Oryza sativa Indica Group, Oryza sativa Japonica Group, Setaria italica, Sorghum bicolor, Avena sativa, and Triticum dicoccoides. In certain embodiments, the plants or plant parts according to the invention as described herein, or generated, produced, obtained, identified, and/or selected according to the invention as described herein, are from the species Triticum aestivum, Aegilops tauschii, Brachypodium distachyon, Hordeum vulgare, Leersia perrieri, Oryza barthii, Oryza brachyantha, Oryza glaberrima, Oryza glumipatula, Oryza longistaminata, Oryza meridionalis, Oryza nivara, Oryza punctata, Oryza rufipogon, Oryza sativa Indica Group, Oryza sativa Japonica Group, Setaria italica, Sorghum bicolor, Avena sativa, and Triticum dicoccoides. In certain embodiments, the plants or plant parts according to the invention as described herein, or generated, produced, obtained, identified, and/or selected according to the invention as described herein, are from the species Triticum aestivum, Aegilops tauschii, Brachypodium distachyon, Hordeum vulgare, Leersia perrieri, Oryza barthii, Oryza brachyantha, Oryza glaberrima, Oryza glumipatula, Oryza longistaminata, Oryza meridionalis, Oryza nivara, Oryza punctata, Oryza rufipogon, Oryza sativa Indica Group, Oryza sativa Japonica Group, Setaria italica, Sorghum bicolor, and Triticum dicoccoides. In certain embodiments, the plants or plant parts according to the invention as described herein, or generated, produced, obtained, identified, and/or selected according to the invention as described herein, are not from the genus Zea. In certain embodiments, the plants or plant parts according to the invention as described herein, or generated, produced, obtained, identified, and/or selected according to the invention as described herein, are not from the species Zea mays. In certain embodiments, the plants or plant parts according to the invention as described herein, or generated, produced, obtained, identified, and/or selected according to the invention as described herein, are not from the species Triticum urartu.

In certain embodiments, the plant or plant part according to the invention as described herein, or generated, produced, obtained, identified, and/or selected according to the invention as described herein, is a crop plant or crop plant part. In certain embodiments, the plant or plant part according to the invention as described herein, or generated, produced, obtained, identified, and/or selected according to the invention as described herein, is a turf grass.

In certain embodiments, the plants or plant parts according to the invention as described herein, or generated, produced, obtained, identified, and/or selected according to the invention as described herein, are from a species comprising the gene pair, such as Myb80 and Dwarf11, on the same chromosome. In certain embodiments, the plants or plant parts according to the invention as described herein, or generated, produced, obtained, identified, and/or selected according to the invention as described herein, are from a species comprising the gene pair, such as Myb80 and Dwarf11, on the same chromosome arm. In certain embodiments, the plants or plant parts according to the invention as described herein, or generated, produced, obtained, identified, and/or selected according to the invention as described herein, are from a species comprising the gene pair, such as Myb80 and Dwarf11, on the same chromosome within 1 cM from each other. In certain embodiments, the plants or plant parts according to the invention as described herein, or generated, produced, obtained, identified, and/or selected according to the invention as described herein, are from a species comprising the gene pair, such as Myb80 and Dwarf11, on the same chromosome within 1 Mbp from each other. In certain embodiments, the plants or plant parts according to the invention as described herein, or generated, produced, obtained, identified, and/or selected according to the invention as described herein, are from a species comprising the gene pair, such as Myb80 and Dwarf11, on the same chromosome arm within 1 cM from each other. In certain embodiments, the plants or plant parts according to the invention as described herein, or generated, produced, obtained, identified, and/or selected according to the invention as described herein, are from a species comprising the gene pair, such as Myb80 and Dwarf11, on the same chromosome arm within 1 Mbp from each other.

In certain embodiments, in the plants or plant parts according to the invention as described herein, or generated, produced, obtained, identified, and/or selected according to the invention as described herein, the genetic distance between the gene pair, such as Myb80 and Dwarf11, is at most 1 cM (on the same chromosome arm). In certain embodiments, in the plants or plant parts according to the invention as described herein, or generated, produced, obtained, identified, and/or selected according to the invention as described herein, the physical distance between the gene pair, such as Myb80 and Dwarf11, is at most 1 Mbp (on the same chromosome arm).

In certain embodiments, the gene pair, such as Myb80 and Dwarf11, are on the same chromosome. In certain embodiments, the gene pair, such as Myb80 and Dwarf11, are on the same chromosome arm. In certain embodiments, the gene pair, such as Myb80 and Dwarf11, are on the same chromosome within 1 cM from each other. In certain embodiments, the gene pair, such as Myb80 and Dwarf11, are on the same chromosome within 1 Mbp from each other. In certain embodiments, the gene pair, such as Myb80 and Dwarf11, are on the same chromosome arm within 1 cM from each other. In certain embodiments, the gene pair, such as Myb80 and Dwarf11, are on the same chromosome arm within 1 Mbp from each other.

In certain embodiments, the genetic distance between the gene pair, such as Myb80 and Dwarf11, is at most 1 cM (are on the same chromosome arm). In certain embodiments, the physical distance between the gene pair, such as Myb80 and Dwarf11, is at most 1 Mbp (are on the same chromosome arm).

In an aspect, the invention relates to a method for developing an assay to (phenotypically) detect a (allele of a) gene of interest in a plant or plant part, comprising screening for the presence of genes located at most 1 Mbp up-of downstream in the chromosome, preferably on the same chromosome arm, comprising said gene of interest, and selecting a gene causing or capable of causing a (allele-dependent or allele-specific) phenotype in a plant or plant part (as a proxy for (i.e. in lieu of) (phenotypically) detecting said gene (allele) of interest).

In an aspect, the invention relates to a method for developing an assay to phenotypically detect a (allele of a) gene of interest in a plant or plant part, comprising screening for the presence of genes located at most 1 Mbp up-of downstream in the chromosome, preferably on the same chromosome arm, comprising said gene of interest, and selecting a gene causing or capable of causing a allele-dependent or allele-specific phenotype in a plant or plant part (as a proxy for (i.e. in lieu of) (phenotypically) detecting said gene (allele) of interest).

In an aspect, the invention relates to a method for developing an assay to (phenotypically) detect a (allele of a) gene of interest in a plant or plant part, comprising screening for the presence of genes located at most 1 cM up-of downstream in the chromosome, preferably on the same chromosome arm, comprising said gene of interest, and selecting a gene causing or capable of causing a (allele-dependent or allele-specific) phenotype in a plant or plant part (as a proxy for (i.e. in lieu of) (phenotypically) detecting said gene (allele) of interest).

In an aspect, the invention relates to a method for developing an assay to phenotypically detect a (allele of a) gene of interest in a plant or plant part, comprising screening for the presence of genes located at most 1 cM up-of downstream in the chromosome, preferably on the same chromosome arm, comprising said gene of interest, and selecting a gene causing or capable of causing a allele-dependent or allele-specific phenotype in a plant or plant part (as a proxy for (i.e. in lieu of) (phenotypically) detecting said gene (allele) of interest).

In certain embodiments, said allele of said gene of interest is recessive. In certain embodiments, said allele-dependent or allele-specific phenotype is recessive. In certain embodiments, said allele of said gene of interest and said allele-dependent or allele-specific phenotype is recessive.

In certain embodiments, said gene of interest is an allele-dependent or allele-specific male or female sterility gene, preferably a male sterility gene. In certain embodiments, said gene of interest is an allele-dependent or allele-specific genetic male or female sterility gene, preferably a genetic male sterility gene.

In certain embodiments, said allele-dependent or allele-specific phenotype is an allele-dependent or allele-specific morphological or colour phenotype or trait, as described herein elsewhere.

Preferably, according to the invention, the identification or selection of plants or plant parts is a phenotypic identification or selection. Plants or plant parts are identified or selected based on a particular phenotype, associated with an underlying genotype. Accordingly, screening for a particular genotype, such as the presence of a particular gene or gene allele, mutation, gene knockout, etc. preferably encompasses phenotypic screening. The present invention relates to gene pairs, in particular linked genes (such as having a genetic distance of at most 1 cM or a physical distance of at most 1 Mbp, as described herein elsewhere), of which one of the genes confers a desired phenotype, such as genetic male or female sterility, as described herein elsewhere, and of which the other gene confers an easily identifiable phenotype, such as dwarfism, as described herein elsewhere. Accordingly, identification or selection based on the easily identifiable phenotype equally identifies or selects the desired phenotype. As used herein, phenotypic screening refers to screening for a particular phenotype. As used herein, phenotypic selection refers to selection of a particular phenotype. As used herein, phenotypic identification refers to identification of a particular phenotype.

In certain embodiments, screening for the presence of, identifying, or selecting a (homozygous) Myb80 mutation (e.g. in a plant or plant part) encompasses screening for the presence of, identifying, or selecting a (homozygous) Dwarf11 mutation (e.g. in a plant or plant part), and vice versa.

In certain embodiments, screening for the presence of, identifying, or selecting genetic male sterility (e.g. in a plant or plant part) encompasses screening for the presence of, identifying, or selecting dwarfism (e.g. in a plant or plant part), and vice versa.

In certain embodiments, screening for the presence of, identifying, or selecting a (homozygous) Myb80 mutation (e.g. in a plant or plant part) encompasses screening for the presence of, identifying, or selecting dwarfism (e.g. in a plant or plant part), and vice versa.

In certain embodiments, screening for the presence of, identifying, or selecting genetic male sterility (e.g. in a plant or plant part) encompasses screening for the presence of, identifying, or selecting a (homozygous) Dwarf11 mutation (e.g. in a plant or plant part), and vice versa.

As used herein, phenotypic screening, selection, or identification may be based on any phenotype, preferably any readily identifiable phenotype. In certain embodiments, phenotypic screening, selection, or identification is based on a morphological phenotype, such as plant height or leaf length, plant or plant part shape, plant part weight, such as seed weight (as can advantageously be determined for instance as 1000 kernel weight), etc. In certain embodiments, phenotypic screening, selection, or identification is based on a colour phenotype, such as plant or plant part colour, for instance seed or grain colour, leaf colour, etc. In certain embodiments, phenotypic screening, selection, or identification is based on plant grain/seed size, grain/seed shape, or grain/seed weight. In certain embodiments, phenotypic screening, selection, or identification is based on plant height. In certain embodiments, phenotypic screening, selection, or identification is based on average plant grain/seed size, grain/seed shape, or grain/seed weight. In certain embodiments, phenotypic screening, selection, or identification is based on average plant height. In certain embodiments, phenotypic screening, selection, or identification is based on individual plant grain/seed size, grain/seed shape, or grain/seed weight. In certain embodiments, phenotypic screening, selection, or identification is based on individual plant height. In certain embodiments, plant height is determined as the height of the plant from the base of the culm or from the crown to the tip of the flag leaf or the tip of the flower/inflorescence/panicle/raceme/spike/seedhead.

In certain embodiments plants, such as genetic male sterile plants, are selected which are at least 5%, preferably at least 8%, more preferably at least 10% shorter than plants which are not genetic male sterile. As a reference, the plant height can be compared to average plant height of plants which are not genetic male sterile. In certain embodiments plants, such as genetic male sterile plants, are selected of which the height is at least 5%, preferably at least 8%, more preferably at least 10% less than the average height of plants which are not genetic male sterile (e.g. plants not having a (homozygous) Myb80 (knockout) mutation).

In certain embodiments plants, such as dwarfism plants, are selected which are at least 5%, preferably at least 8%, more preferably at least 10% shorter than plants which do not exhibit dwarfism. As a reference, the plant height can be compared to average plant height of plants which do not exhibit dwarfism. In certain embodiments plants, such as genetic male sterile plants, are selected of which the height is at least 5%, preferably at least 8%, more preferably at least 10% less than the average height of plants which do not exhibit dwarfism (e.g. plants not having a (homozygous) Dwarf11 (knockout) mutation).

In certain embodiments seeds of plants, such as seeds of genetic male sterile plants, are selected which are at least 5%, preferably at least 8%, more preferably at least 10% shorter (i.e. have a shorter length) than seeds of plants which are not genetic male sterile. As a reference, the seed length can be compared to average seed length of seeds from plants which are not genetic male sterile. In certain embodiments seeds of plants, such as seeds of genetic male sterile plants, are selected of which the length is at least 5%, preferably at least 8%, more preferably at least 10% less than the average length of seeds of plants which are not genetic male sterile (e.g. plants not having a (homozygous) Myb80 (knockout) mutation).

In certain embodiments seeds of plants, such as seeds of dwarfism plants, are selected which are at least 5%, preferably at least 8%, more preferably at least 10% shorter (i.e. have a shorter length) than seeds of plants which do not exhibit dwarfism. As a reference, the seed length can be compared to average seed length of plants which do not exhibit dwarfism. In certain embodiments seeds of plants, such as seeds of genetic male sterile plants, are selected of which the length is at least 5%, preferably at least 8%, more preferably at least 10% less than the average length of seeds of plants which do not exhibit dwarfism (e.g. plants not having a (homozygous) Dwarf11 (knockout) mutation).

In certain embodiments seeds of plants, such as seeds of genetic male sterile plants, are selected which are at least 5%, preferably at least 8%, more preferably at least 10% lighter (i.e. have a lower weight) than seeds of plants which are not genetic male sterile. As a reference, the seed weight can be compared to average seed weight of seeds from plants which are not genetic male sterile. In certain embodiments seeds of plants, such as seeds of genetic male sterile plants, are selected of which the weight is at least 5%, preferably at least 8%, more preferably at least 10% less than the average weight of seeds of plants which are not genetic male sterile (e.g. plants not having a (homozygous) Myb80 (knockout) mutation).

In certain embodiments seeds of plants, such as seeds of dwarfism plants, are selected which are at least 4%, preferably at least 6%, more preferably at least 8% lighter (i.e. have a lower weight) than seeds of plants which do not exhibit dwarfism. As a reference, the seed weight can be compared to average seed weight of plants which do not exhibit dwarfism. In certain embodiments seeds of plants, such as seeds of genetic male sterile plants, are selected of which the weight is at least 4%, preferably at least 6%, more preferably at least 8% less than the average weight of seeds of plants which do not exhibit dwarfism (e.g. plants not having a (homozygous) Dwarf11 (knockout) mutation).

In certain embodiments plants, such as genetic male sterile plants, are selected which have a plant height less than a threshold. As a threshold, the plant height can be the average plant height of (reference) plants which are not genetic male sterile. In certain embodiments plants, such as genetic male sterile plants, are selected of which the height is less than a threshold, which is the average height of (reference) plants which are not genetic male sterile (e.g. plants not having a (homozygous) Myb80 (knockout) mutation).

In certain embodiments plants, such as dwarfism plants, are selected which have a height less than a threshold. As a threshold, the plant height can be the average plant height of (reference) plants which do not exhibit dwarfism. In certain embodiments plants, such as genetic male sterile plants, are selected of which the height is less than a threshold, which is the average height of (reference) plants which do not exhibit dwarfism (e.g. plants not having a (homozygous) Dwarf11 (knockout) mutation).

In certain embodiments seeds of plants, such as genetic male sterile plants, are selected which have a seed length less than a threshold. As a threshold, the seed length can be the average seed length of (reference) plants which are not genetic male sterile. In certain embodiments seeds of plants, such as seeds of genetic male sterile plants, are selected of which the seed length is less than a threshold, which is the average seed lengths of (reference) plants which are not genetic male sterile (e.g. plants not having a (homozygous) Myb80 (knockout) mutation).

In certain embodiments seeds of plants, such as dwarfism plants, are selected which have a seed length less than a threshold. As a threshold, the seed length can be the average seed length of (reference) plants which do not exhibit dwarfism. In certain embodiments seeds of plants, such as seeds of genetic male sterile plants, are selected of which the seed length is less than a threshold, which is the average seed length of (reference) plants which do not exhibit dwarfism (e.g. plants not having a (homozygous) Dwarf11 (knockout) mutation).

In certain embodiments seeds of plants, such as genetic male sterile plants, are selected which have a seed weight less than a threshold. As a threshold, the seed weight can be the average seed weight of (reference) plants which are not genetic male sterile. In certain embodiments seeds of plants, such as seeds of genetic male sterile plants, are selected of which the seed weight is less than a threshold, which is the average seed weight of (reference) plants which are not genetic male sterile (e.g. plants not having a (homozygous) Myb80 (knockout) mutation).

In certain embodiments seeds of plants, such as dwarfism plants, are selected which have a seed weight less than a threshold. As a threshold, the seed weight can be the average seed weight of (reference) plants which do not exhibit dwarfism. In certain embodiments seeds of plants, such as seeds of genetic male sterile plants, are selected of which the seed weight is less than a threshold, which is the average seed weight of (reference) plants which do not exhibit dwarfism (e.g. plants not having a (homozygous) Dwarf11 (knockout) mutation).

As a reference plant or plant part, an isogenic line may be used, i.e. a plant otherwise identical apart from the (homozygous) Dwarf11 and/or Myb80 (knockout) mutation, as referred to herein elsewhere.

In certain embodiments, the plants or plant parts according to the invention as described herein, or generated, produced, obtained, identified, and/or selected according to the invention as described herein can be used in hybrid breeding or for generating hybrid plants or plant part, such as hybrid seeds. Accordingly, in an aspect, the invention relates to the use of the plants or plant parts according to the invention as described herein, or generated, produced, obtained, identified, and/or selected according to the invention as described herein in hybrid breeding, or for generating hybrid plants or plant parts, such as hybrid seeds.

In an aspect, the invention relates to a polynucleotide having, comprising, consisting (essentially) of, or comprised in or encoding a sequence as set forth in any of SEQ ID NOs: 1-392, a (unique) fragment thereof, or the complement or reverse complement thereof. In an aspect, the invention relates to a polynucleotide having, comprising, consisting (essentially) of, or comprised in or encoding a sequence which is at least 80%, at least 85%, at least 90%, at least 95%, at least 96%), at least 97%, at least 98%, or at least 99% identical to a sequence as set forth in any of SEQ ID NOs: 1-392, a (unique) fragment thereof, or the complement or reverse complement thereof. Fragments preferably are at least 15 nucleotides, more preferably at least 18 nucleotides, most preferably at least 20 nucleotides.

In an aspect, the invention relates to a polynucleotide having, comprising, consisting (essentially) of, or comprised in or encoding a sequence as set forth in any of SEQ ID NOs: 1-4 or 115-392, a (unique) fragment thereof, or the complement or reverse complement thereof. In an aspect, the invention relates to a polynucleotide having, comprising, consisting (essentially) of, or comprised in or encoding a sequence which is at least 80%, at least 85%, at least 90%, at least 95%, at least 96%), at least 97%, at least 98%, or at least 99% identical to a sequence as set forth in any of SEQ ID NOs: 1-4 or 115-392, a (unique) fragment thereof, or the complement or reverse complement thereof. Fragments preferably are at least 15 nucleotides, more preferably at least 18 nucleotides, most preferably at least 20 nucleotides.

In an aspect, the invention relates to a polynucleotide having, comprising, consisting (essentially) of, or comprised in or encoding a sequence as set forth in any of SEQ ID NOs: 5-114, a (unique) fragment thereof, or the complement or reverse complement thereof. In an aspect, the invention relates to a polynucleotide having, comprising, consisting (essentially) of, or comprised in or encoding a sequence which is at least 80%, at least 85%, at least 90%, at least 95%, at least 96%), at least 97%, at least 98%, or at least 99% identical to a sequence as set forth in any of SEQ ID NOs: 5-114, a (unique) fragment thereof, or the complement or reverse complement thereof. Fragments preferably are at least 15 nucleotides, more preferably at least 18 nucleotides, most preferably at least 20 nucleotides. In certain embodiments, the polynucleotide is or is comprised in a guide RNA (gRNA), preferably a CRISPR/Cas guide RNA (gRNA). In an aspect, the invention relates to a guide RNA (gRNA), preferably a CRISPR/Cas guide RNA (gRNA) comprising a (spacer) sequence as set forth in any of SEQ ID NOs: 5-114. In an aspect, the invention relates to a guide RNA (gRNA), preferably a CRISPR/Cas guide RNA (gRNA) comprising a (spacer) sequence which is at least 80%, at least 85%, at least 90%, at least 95%, at least 96%), at least 97%, at least 98%, or at least 99% identical to a sequence as set forth in any of SEQ ID NOs: 5-114.

In an aspect, the invention relates to a polynucleotide having, comprising, consisting (essentially) of, or comprised in or encoding a sequence as set forth in any of SEQ ID NOs: 5-95, a (unique) fragment thereof, or the complement or reverse complement thereof. In an aspect, the invention relates to a polynucleotide having, comprising, consisting (essentially) of, or comprised in or encoding a sequence which is at least 80%, at least 85%, at least 90%, at least 95%, at least 96%), at least 97%, at least 98%, or at least 99% identical to a sequence as set forth in any of SEQ ID NOs: 5-95, a (unique) fragment thereof, or the complement or reverse complement thereof. Fragments preferably are at least 15 nucleotides, more preferably at least 18 nucleotides, most preferably at least 20 nucleotides. In certain embodiments, the polynucleotide is or is comprised in a guide RNA (gRNA), preferably a CRISPR/Cas guide RNA (gRNA). In an aspect, the invention relates to a guide RNA (gRNA), preferably a CRISPR/Cas guide RNA (gRNA) comprising a (spacer) sequence as set forth in any of SEQ ID NOs: 5-95. In an aspect, the invention relates to a guide RNA (gRNA), preferably a CRISPR/Cas guide RNA (gRNA) comprising a (spacer) sequence which is at least 80%, at least 85%, at least 90%, at least 95%, at least 96%), at least 97%, at least 98%, or at least 99% identical to a sequence as set forth in any of SEQ ID NOs: 5-95.

In an aspect, the invention relates to a polynucleotide having, comprising, consisting (essentially) of, or comprised in or encoding a sequence as set forth in any of SEQ ID NOs: 96-114, a (unique) fragment thereof, or the complement or reverse complement thereof. In an aspect, the invention relates to a polynucleotide having, comprising, consisting (essentially) of, or comprised in or encoding a sequence which is at least 80%, at least 85%, at least 90%, at least 95%, at least 96%), at least 97%, at least 98%, or at least 99% identical to a sequence as set forth in any of SEQ ID NOs: 96-114, a (unique) fragment thereof, or the complement or reverse complement thereof. Fragments preferably are at least 15 nucleotides, more preferably at least 18 nucleotides, most preferably at least 20 nucleotides. In certain embodiments, the polynucleotide is or is comprised in a guide RNA (gRNA), preferably a CRISPR/Cas guide RNA (gRNA). In an aspect, the invention relates to a guide RNA (gRNA), preferably a CRISPR/Cas guide RNA (gRNA) comprising a (spacer) sequence as set forth in any of SEQ ID NOs: 96-114. In an aspect, the invention relates to a guide RNA (gRNA), preferably a CRISPR/Cas guide RNA (gRNA) comprising a (spacer) sequence which is at least 80%, at least 85%, at least 90%, at least 95%, at least 96%), at least 97%, at least 98%, or at least 99% identical to a sequence as set forth in any of SEQ ID NOs: 96-114.

In an aspect, the invention relates to a polynucleotide having, comprising, consisting (essentially) of, or comprised in or encoding a sequence as set forth in any of SEQ ID NOs: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 162, 163, 164, 169, 170, 171, 175, 176, 177, 178, 179, 180, 181, 186, 187, 188, 189, 190, 191, 192, 193, 198, 199, 200, 201, 206, 207, 208, 209, 214, 215, 216, 217, 226, 227, 228, 229, 234, 235, 236, 237, 254, 255, 256, 257, 262, 263, 264, 265, 270, 271, 272, 273, 278, 279, 280, 281, 282, 283, 284, 289, 290, 291, 292, 297, 298, 299, 300, 305, 306, 307, 308, 309, 310, 311, 312, 313, 314, 315, 316, 317, 318, 319, 320, 321, 322, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 377, 378, 379, 380, 385, 386, 387, or 388, a (unique) fragment thereof, or the complement or reverse complement thereof. In an aspect, the invention relates to a polynucleotide having, comprising, consisting (essentially) of, or comprised in or encoding a sequence which is at least 80%, at least 85%, at least 90%, at least 95%, at least 96%), at least 97%, at least 98%, or at least 99% identical to a sequence as set forth in any of SEQ ID NOS: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 162, 163, 164, 169, 170, 171, 175, 176, 177, 178, 179, 180, 181, 186, 187, 188, 189, 190, 191, 192, 193, 198, 199, 200, 201, 206, 207, 208, 209, 214, 215, 216, 217, 226, 227, 228, 229, 234, 235, 236, 237, 254, 255, 256, 257, 262, 263, 264, 265, 270, 271, 272, 273, 278, 279, 280, 281, 282, 283, 284, 289, 290, 291, 292, 297, 298, 299, 300, 305, 306, 307, 308, 309, 310, 311, 312, 313, 314, 315, 316, 317, 318, 319, 320, 321, 322, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 377, 378, 379, 380, 385, 386, 387, or 388, a (unique) fragment thereof, or the complement or reverse complement thereof. Fragments preferably are at least 15 nucleotides, more preferably at least 18 nucleotides, most preferably at least 20 nucleotides.

In an aspect, the invention relates to a polynucleotide having, comprising, consisting (essentially) of, or comprised in or encoding a sequence as set forth in any of SEQ ID NOs: 152, 153, 154, 155, 156, 157, 158, 159, 160, 161, 165, 166, 167, 168, 172, 173, 174, 182, 183, 184, 185, 194, 195, 196, 197, 202, 203, 204, 205, 210, 211, 212, 213, 218, 219, 220, 221, 222, 223, 224, 225, 230, 231, 232, 233, 238, 239, 240, 241, 242, 243, 244, 245, 246, 247, 248, 249, 250, 251, 252, 253, 258, 259, 260, 261, 266, 267, 268, 269, 274, 275, 276, 277, 285, 286, 287, 288, 293, 294, 295, 296, 301, 302, 303, 304, 323, 324, 325, 326, 327, 328, 329, 330, 331, 332, 333, 334, 373, 374, 375, 376, 381, 382, 383, 384, 389, 390, 391, or 392, a (unique) fragment thereof, or the complement or reverse complement thereof. In an aspect, the invention relates to a polynucleotide having, comprising, consisting (essentially) of, or comprised in or encoding a sequence which is at least 80%, at least 85%, at least 90%, at least 95%, at least 96%), at least 97%, at least 98%, or at least 99% identical to a sequence as set forth in any of SEQ ID NOs: 152, 153, 154, 155, 156, 157, 158, 159, 160, 161, 165, 166, 167, 168, 172, 173, 174, 182, 183, 184, 185, 194, 195, 196, 197, 202, 203, 204, 205, 210, 211, 212, 213, 218, 219, 220, 221, 222, 223, 224, 225, 230, 231, 232, 233, 238, 239, 240, 241, 242, 243, 244, 245, 246, 247, 248, 249, 250, 251, 252, 253, 258, 259, 260, 261, 266, 267, 268, 269, 274, 275, 276, 277, 285, 286, 287, 288, 293, 294, 295, 296, 301, 302, 303, 304, 323, 324, 325, 326, 327, 328, 329, 330, 331, 332, 333, 334, 373, 374, 375, 376, 381, 382, 383, 384, 389, 390, 391, or 392, a (unique) fragment thereof, or the complement or reverse complement thereof. Fragments preferably are at least 15 nucleotides, more preferably at least 18 nucleotides, most preferably at least 20 nucleotides.

In an aspect, the invention relates to a polypeptide having, comprising, consisting (essentially) of, or comprised in a sequence as set forth in any of SEQ ID NOs: 117, 120, 123, 126, 129, 132, 135, 138, 141, 144, 147, 150, 154, 157, 160, 163, 167, 170, 173, 177, 180, 184, 188, 192, 196, 200, 204, 208, 212, 216, 220, 224, 228, 232, 236, 240, 243, 246, 249, 252, 256, 260, 264, 268, 272, 276, 280, 283, 287, 291, 295, 299, 303, 307, 310, 314, 317, 321, 325, 329, 333, 337, 340, 343, 346, 349, 352, 355, 358, 362, 365, 368, 371, 375, 379, 383, 387, or 391, or a (unique) fragment thereof. In an aspect, the invention relates to a polypeptide having, comprising, consisting (essentially) of, or comprised in a sequence which is at least 80%, at least 85%, at least 90%, at least 95%, at least 96%), at least 97%, at least 98%, or at least 99% identical to a sequence as set forth in any of SEQ ID NOs: 117, 120, 123, 126, 129, 132, 135, 138, 141, 144, 147, 150, 154, 157, 160, 163, 167, 170, 173, 177, 180, 184, 188, 192, 196, 200, 204, 208, 212, 216, 220, 224, 228, 232, 236, 240, 243, 246, 249, 252, 256, 260, 264, 268, 272, 276, 280, 283, 287, 291, 295, 299, 303, 307, 310, 314, 317, 321, 325, 329, 333, 337, 340, 343, 346, 349, 352, 355, 358, 362, 365, 368, 371, 375, 379, 383, 387, or 391, or a (unique) fragment thereof. Fragments preferably are at least 15 amino acids, more preferably at least 18 amino acids, most preferably at least 20 amino acids.

In an aspect, the invention relates to a polynucleotide encoding a polypeptide having a sequence as set forth in any of SEQ ID NOs: 117, 120, 123, 126, 129, 132, 135, 138, 141, 144, 147, 150, 154, 157, 160, 163, 167, 170, 173, 177, 180, 184, 188, 192, 196, 200, 204, 208, 212, 216, 220, 224, 228, 232, 236, 240, 243, 246, 249, 252, 256, 260, 264, 268, 272, 276, 280, 283, 287, 291, 295, 299, 303, 307, 310, 314, 317, 321, 325, 329, 333, 337, 340, 343, 346, 349, 352, 355, 358, 362, 365, 368, 371, 375, 379, 383, 387, or 391, a (unique) fragment thereof, or the complement or reverse complement thereof. In an aspect, the invention relates to a polynucleotide encoding a polypeptide having a sequence which is at least 80%, at least 85%, at least 90%, at least 95%, at least 96%), at least 97%, at least 98%, or at least 99% identical to a sequence as set forth in any of SEQ ID NOs: 117, 120, 123, 126, 129, 132, 135, 138, 141, 144, 147, 150, 154, 157, 160, 163, 167, 170, 173, 177, 180, 184, 188, 192, 196, 200, 204, 208, 212, 216, 220, 224, 228, 232, 236, 240, 243, 246, 249, 252, 256, 260, 264, 268, 272, 276, 280, 283, 287, 291, 295, 299, 303, 307, 310, 314, 317, 321, 325, 329, 333, 337, 340, 343, 346, 349, 352, 355, 358, 362, 365, 368, 371, 375, 379, 383, 387, or 391, a (unique) fragment thereof, or the complement or reverse complement thereof. Fragments preferably are at least 15 nucleotides, more preferably at least 18 nucleotides, most preferably at least 20 nucleotides.

In an aspect, the invention relates to a polypeptide having, comprising, consisting (essentially) of, or comprised in a sequence as set forth in any of SEQ ID NOs: 117, 120, 123, 126, 129, 132, 135, 138, 141, 144, 147, 150, 163, 170, 177, 180, 188, 192, 200, 208, 216, 228, 236, 256, 264, 272, 280, 283, 291, 299, 307, 310, 314, 317, 321, 337, 340, 343, 346, 349, 352, 355, 358, 362, 365, 368, 371, 379, or 387, or a (unique) fragment thereof. In an aspect, the invention relates to a polypeptide having, comprising, consisting (essentially) of, or comprised in a sequence which is at least 80%, at least 85%, at least 90%, at least 95%, at least 96%), at least 97%, at least 98%, or at least 99% identical to a sequence as set forth in any of SEQ ID NOs: 117, 120, 123, 126, 129, 132, 135, 138, 141, 144, 147, 150, 163, 170, 177, 180, 188, 192, 200, 208, 216, 228, 236, 256, 264, 272, 280, 283, 291, 299, 307, 310, 314, 317, 321, 337, 340, 343, 346, 349, 352, 355, 358, 362, 365, 368, 371, 379, or 387, or a (unique) fragment thereof. Fragments preferably are at least 15 amino acids, more preferably at least 18 amino acids, most preferably at least 20 amino acids.

In an aspect, the invention relates to a polynucleotide encoding a polypeptide having a sequence as set forth in any of SEQ ID NOs: 117, 120, 123, 126, 129, 132, 135, 138, 141, 144, 147, 150, 163, 170, 177, 180, 188, 192, 200, 208, 216, 228, 236, 256, 264, 272, 280, 283, 291, 299, 307, 310, 314, 317, 321, 337, 340, 343, 346, 349, 352, 355, 358, 362, 365, 368, 371, 379, or 387, a (unique) fragment thereof, or the complement or reverse complement thereof. In an aspect, the invention relates to a polynucleotide encoding a polypeptide having a sequence which is at least 80%, at least 85%, at least 90%, at least 95%, at least 96%), at least 97%, at least 98%, or at least 99% identical to a sequence as set forth in any of SEQ ID NOs: 117, 120, 123, 126, 129, 132, 135, 138, 141, 144, 147, 150, 163, 170, 177, 180, 188, 192, 200, 208, 216, 228, 236, 256, 264, 272, 280, 283, 291, 299, 307, 310, 314, 317, 321, 337, 340, 343, 346, 349, 352, 355, 358, 362, 365, 368, 371, 379, or 387, a (unique) fragment thereof, or the complement or reverse complement thereof. Fragments preferably are at least 15 nucleotides, more preferably at least 18 nucleotides, most preferably at least 20 nucleotides.

In an aspect, the invention relates to a polypeptide having, comprising, consisting (essentially) of, or comprised in a sequence as set forth in any of SEQ ID NOs: 154, 157, 160, 167, 173, 184, 196, 204, 212, 220, 224, 232, 240, 243, 246, 249, 252, 260, 268, 276, 287, 295, 303, 325, 329, 333, 375, 383, or 391, or a (unique) fragment thereof. In an aspect, the invention relates to a polypeptide having, comprising, consisting (essentially) of, or comprised in a sequence which is at least 80%, at least 85%, at least 90%, at least 95%, at least 96%), at least 97%, at least 98%, or at least 99% identical to a sequence as set forth in any of SEQ ID NOs: 154, 157, 160, 167, 173, 184, 196, 204, 212, 220, 224, 232, 240, 243, 246, 249, 252, 260, 268, 276, 287, 295, 303, 325, 329, 333, 375, 383, or 391, or a (unique) fragment thereof. Fragments preferably are at least 15 amino acids, more preferably at least 18 amino acids, most preferably at least 20 amino acids.

In an aspect, the invention relates to a polynucleotide encoding a polypeptide having a sequence as set forth in any of SEQ ID NOs: 154, 157, 160, 167, 173, 184, 196, 204, 212, 220, 224, 232, 240, 243, 246, 249, 252, 260, 268, 276, 287, 295, 303, 325, 329, 333, 375, 383, or 391, a (unique) fragment thereof, or the complement or reverse complement thereof. In an aspect, the invention relates to a polynucleotide encoding a polypeptide having a sequence which is at least 80%, at least 85%, at least 90%, at least 95%, at least 96%), at least 97%, at least 98%, or at least 99% identical to a sequence as set forth in any of SEQ ID NOs: 154, 157, 160, 167, 173, 184, 196, 204, 212, 220, 224, 232, 240, 243, 246, 249, 252, 260, 268, 276, 287, 295, 303, 325, 329, 333, 375, 383, or 391, a (unique) fragment thereof, or the complement or reverse complement thereof. Fragments preferably are at least 15 nucleotides, more preferably at least 18 nucleotides, most preferably at least 20 nucleotides.

In certain embodiments, the Dwarf11 gene, coding sequence, or protein as referred to herein has, comprises, consists (essentially) of, or is comprised in a sequence as set forth in in any of SEQ ID NOs: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 162, 163, 164, 169, 170, 171, 175, 176, 177, 178, 179, 180, 181, 186, 187, 188, 189, 190, 191, 192, 193, 198, 199, 200, 201, 206, 207, 208, 209, 214, 215, 216, 217, 226, 227, 228, 229, 234, 235, 236, 237, 254, 255, 256, 257, 262, 263, 264, 265, 270, 271, 272, 273, 278, 279, 280, 281, 282, 283, 284, 289, 290, 291, 292, 297, 298, 299, 300, 305, 306, 307, 308, 309, 310, 311, 312, 313, 314, 315, 316, 317, 318, 319, 320, 321, 322, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 377, 378, 379, 380, 385, 386, 387, or 388. In certain embodiments, the Dwarf11 gene, coding sequence, or protein as referred to herein has, comprises, consists (essentially) of, or is comprised in a sequence which is at least 80%, at least 85%, at least 90%, at least 95%, at least 96%), at least 97%, at least 98%, or at least 99% identical to a sequence as set forth in any of SEQ ID NOs: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 162, 163, 164, 169, 170, 171, 175, 176, 177, 178, 179, 180, 181, 186, 187, 188, 189, 190, 191, 192, 193, 198, 199, 200, 201, 206, 207, 208, 209, 214, 215, 216, 217, 226, 227, 228, 229, 234, 235, 236, 237, 254, 255, 256, 257, 262, 263, 264, 265, 270, 271, 272, 273, 278, 279, 280, 281, 282, 283, 284, 289, 290, 291, 292, 297, 298, 299, 300, 305, 306, 307, 308, 309, 310, 311, 312, 313, 314, 315, 316, 317, 318, 319, 320, 321, 322, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 377, 378, 379, 380, 385, 386, 387, or 388.

In certain embodiments, the Myb80 gene, coding sequence, or protein as referred to herein has, comprises, consists (essentially) of, or is comprised in a sequence as set forth in in any of SEQ ID NOs: 152, 153, 154, 155, 156, 157, 158, 159, 160, 161, 165, 166, 167, 168, 172, 173, 174, 182, 183, 184, 185, 194, 195, 196, 197, 202, 203, 204, 205, 210, 211, 212, 213, 218, 219, 220, 221, 222, 223, 224, 225, 230, 231, 232, 233, 238, 239, 240, 241, 242, 243, 244, 245, 246, 247, 248, 249, 250, 251, 252, 253, 258, 259, 260, 261, 266, 267, 268, 269, 274, 275, 276, 277, 285, 286, 287, 288, 293, 294, 295, 296, 301, 302, 303, 304, 323, 324, 325, 326, 327, 328, 329, 330, 331, 332, 333, 334, 373, 374, 375, 376, 381, 382, 383, 384, 389, 390, 391, or 392. In certain embodiments, the Myb80 gene, coding sequence, or protein as referred to herein has, comprises, consists (essentially) of, or is comprised in a sequence which is at least 80%, at least 85%, at least 90%, at least 95%, at least 96%), at least 97%, at least 98%, or at least 99% identical to a sequence as set forth in any of SEQ ID NOs: 152, 153, 154, 155, 156, 157, 158, 159, 160, 161, 165, 166, 167, 168, 172, 173, 174, 182, 183, 184, 185, 194, 195, 196, 197, 202, 203, 204, 205, 210, 211, 212, 213, 218, 219, 220, 221, 222, 223, 224, 225, 230, 231, 232, 233, 238, 239, 240, 241, 242, 243, 244, 245, 246, 247, 248, 249, 250, 251, 252, 253, 258, 259, 260, 261, 266, 267, 268, 269, 274, 275, 276, 277, 285, 286, 287, 288, 293, 294, 295, 296, 301, 302, 303, 304, 323, 324, 325, 326, 327, 328, 329, 330, 331, 332, 333, 334, 373, 374, 375, 376, 381, 382, 383, 384, 389, 390, 391, or 392.

In certain embodiments, Dwarf11 comprises or consists (essentially) of a coding sequence as set forth in in any of SEQ ID NOs: 116, 119, 122, 125, 128, 131, 134, 137, 140, 143, 146, 149, 176, 179, 187, 191, 199, 207, 215, 227, 235, 255, 263, 271, 279, 282, 290, 298, 306, 309, 313, 316, 320, 336, 339, 342, 345, 348, 351, 354, 357, 361, 364, 367, 370, 378, or 386. In certain embodiments, Dwarf11 comprises or consists (essentially) of a coding sequence which is at least 80%, at least 85%, at least 90%, at least 95%, at least 96%), at least 97%, at least 98%, or at least 99% identical to a sequence as set forth in any of SEQ ID NOs: 116, 119, 122, 125, 128, 131, 134, 137, 140, 143, 146, 149, 176, 179, 187, 191, 199, 207, 215, 227, 235, 255, 263, 271, 279, 282, 290, 298, 306, 309, 313, 316, 320, 336, 339, 342, 345, 348, 351, 354, 357, 361, 364, 367, 370, 378, or 386.

In certain embodiments, Dwarf11 comprises or consists (essentially) of a genomic sequence as set forth in in any of SEQ ID NOs: 151, 164, 171, 181, 189, 193, 201, 209, 217, 229, 237, 257, 265, 273, 284, 292, 300, 311, 318, 322, 359, 372, 380, or 388. In certain embodiments, Dwarf11 comprises or consists (essentially) of a genomic sequence which is at least 80%, at least 85%, at least 90%, at least 95%, at least 96%), at least 97%, at least 98%, or at least 99% identical to a sequence as set forth in any of SEQ ID NOs: 151, 164, 171, 181, 189, 193, 201, 209, 217, 229, 237, 257, 265, 273, 284, 292, 300, 311, 318, 322, 359, 372, 380, or 388.

In certain embodiments, Dwarf11 comprises or consists (essentially) of a protein sequence as set forth in in any of SEQ ID NOs: 117, 120, 123, 126, 129, 132, 135, 138, 141, 144, 147, 150, 163, 170, 177, 180, 188, 192, 200, 208, 216, 228, 236, 256, 264, 272, 280, 283, 291, 299, 307, 310, 314, 317, 321, 337, 340, 343, 346, 349, 352, 355, 358, 362, 365, 368, 371, 379, or 387. In certain embodiments, Dwarf11 comprises or consists (essentially) of a protein sequence which is at least 80%, at least 85%, at least 90%, at least 95%, at least 96%), at least 97%, at least 98%, or at least 99% identical to a sequence as set forth in any of SEQ ID NOs: 117, 120, 123, 126, 129, 132, 135, 138, 141, 144, 147, 150, 163, 170, 177, 180, 188, 192, 200, 208, 216, 228, 236, 256, 264, 272, 280, 283, 291, 299, 307, 310, 314, 317, 321, 337, 340, 343, 346, 349, 352, 355, 358, 362, 365, 368, 371, 379, or 387.

In certain embodiments, Myb80 comprises or consists (essentially) of a coding sequence as set forth in in any of SEQ ID NOs: 153, 156, 159, 166, 183, 195, 203, 211, 219, 223, 231, 239, 242, 245, 248, 251, 259, 267, 275, 286, 294, 302, 324, 328, 332, 374, 382, or 390. In certain embodiments, Myb80 comprises or consists (essentially) of a coding sequence which is at least 80%, at least 85%, at least 90%, at least 95%, at least 96%), at least 97%, at least 98%, or at least 99% identical to a sequence as set forth in any of SEQ ID NOs: 153, 156, 159, 166, 183, 195, 203, 211, 219, 223, 231, 239, 242, 245, 248, 251, 259, 267, 275, 286, 294, 302, 324, 328, 332, 374, 382, or 390.

In certain embodiments, Myb80 comprises or consists (essentially) of a genomic sequence as set forth in in any of SEQ ID NOs: 161, 168, 174, 185, 197, 205, 213, 221, 225, 233, 253, 261, 269, 277, 288, 296, 304, 326, 330, 334, 376, 384, or 392. In certain embodiments, Myb80 comprises or consists (essentially) of a genomic sequence which is at least 80%, at least 85%, at least 90%, at least 95%, at least 96%), at least 97%, at least 98%, or at least 99% identical to a sequence as set forth in any of SEQ ID NOs: 161, 168, 174, 185, 197, 205, 213, 221, 225, 233, 253, 261, 269, 277, 288, 296, 304, 326, 330, 334, 376, 384, or 392.

In certain embodiments, Myb80 comprises or consists (essentially) of a protein sequence as set forth in in any of SEQ ID NOs: 154, 157, 160, 167, 173, 184, 196, 204, 212, 220, 224, 232, 240, 243, 246, 249, 252, 260, 268, 276, 287, 295, 303, 325, 329, 333, 375, 383, or 391. In certain embodiments, Myb80 comprises or consists (essentially) of a protein sequence which is at least 80%, at least 85%, at least 90%, at least 95%, at least 96%), at least 97%, at least 98%, or at least 99% identical to a sequence as set forth in any of SEQ ID NOs: 154, 157, 160, 167, 173, 184, 196, 204, 212, 220, 224, 232, 240, 243, 246, 249, 252, 260, 268, 276, 287, 295, 303, 325, 329, 333, 375, 383, or 391.

The skilled person will understand that according to certain embodiments the Dwarf11 and/or Myb80 gene may be mutated, such as knocked out, as a consequence of which the sequence will have been altered.

The present invention is not to be limited in scope by the specific embodiments described herein. Indeed, various modifications of the invention in addition to those described herein will become apparent to those skilled in the art from the foregoing description. Such modifications are intended to fall under the scope of the appended claims. It is further to be understood that all values may include approximates and are provided for description.

The aspects and embodiments of the invention are further supported by the following non-limiting examples. The following examples, including the experiments conducted and the results achieved, are provided for illustrative purposes only and are not constructed as limiting the present invention.

### EXAMPLES

The wheat (Triticum aestivum) genome was screened for a combination of candidate genes conferring either a male sterile phenotype or a morphological phenotype. The screening parameters were set, that the physical distance between the two genes should be less than 1 Mbp. The assumption was that this physical distance is sufficient for a close genetic linkage of the two genes, so they are supposed to be inherited as one genetic locus. Based on experiences, a physical distance of 1 Mbp would result in a genetic distance of <1 cM, meaning that the two loci are only inherited independently in less than 1%, which is negligible and in any case can still be counterselected through the use of molecular markers.

Closely linked Myb80 and Dwarf11 genes were identified (non-exhaustively) in wheat and several other species of the Poaceae family, as indicated in Table 1. Genes were identified using blast search with dwarf11 and myb 103 sequences (in EnsemblePlant, Plaza, or Phytozome, etc.) against all available monocot species with the reference versions pointed out in column “assembly”. In each of the species, both genes were located on the same chromosome within one Mb from each other. Therefore the two genes could be used as general tool for the development of hybrid systems in all cereal plant species. The minimal distance observed in the analyzed cereal plant species was 0.03 Mbp in Setaria italica whereas the maximum observed distance was 0.7 Mbp in Barley (Hordeum vulgare).

TABLE 1

| | | MYB80 | dwarf11 | | Distance |
|---|---|---|---|---|---|
| Species | Assembly | homologue | homologue | Chromosome | (Mb) |
| Aegilops | Aet_v4.0 | AET2Gv20749000 | AET2Gv20748100 | 2D | 0.17 |
| tauschii | | | | | |
| Brachypodium | Brachypodium_distachyon_v3.0 | BRADI_5g13041v3 | BRADI_5g12990v3 | 5 | 0.06 |
| distachyon | | | | | |
| Hordeum | BA_Hullen_v2 | HOVUSG0945200 | HOVUSG0946300 | scaffold | 0.7 |
| vulgare | | | | 67 | |
| Leersia perrieri | Lperr_V1.4 | LPERR04G12420 | LPERR04G12330 | 4 | 0.05 |
| Oryza | Oryza_brachyantha.v1.4b | OB04G23000 | OB04G22900 | 4 | 0.07 |
| brachyantha | | | | | |
| Oryza | Oryza_glaberrima_V1 | ORGLA04G0126700 | ORGLA04G0126200 | 4 | 0.04 |
| glaberrima | | | | | |
| Oryza | Oryza_glumaepatula_v1.5 | OGLUM04G15520 | OGLUM04G15480 | 4 | 0.04 |
| glumipatula | | | | | |
| Oryza | O_longistaminata_v1.0 | KN538714.1_FG046 | KN538714.1_FG040 | 1 | 0.04 |
| longistaminata | | | | | |
| Oryza | Oryza_meridionalis_v1.3 | OMERI04G13500 | OMERI04G13440 | 4 | 0.07 |
| meridionalis | | | | | |
| Oryza nivara | Oryza_nivara_v1.0 | ONIVA04G13800 | ONIVA04G13750 | 4 | 0.04 |
| Oryza punctata | Oryza_punctata_v1.2 | OPUNC04G13560 | OPUNC04G13510 | 4 | 0.04 |
| Oryza | OR_W1943 | ORUFI04G17000 | ORUFI04G16950 | 4 | 0.04 |
| rufipogon | | | | | |
| Oryza sativa | ASM465v1 | BGIOSGA014912 | BGIOSGA014915 | 4 | 0.04 |
| Indica Group | | | | | |
| Oryza sativa | IRGSP-1.0 | Os04g0470600 | Os04g0469800 | 4 | 0.04 |
| Japonica Group | | | | | |
| Setaria italica | Setaria_italica_v2.0 | SETIT_012130mg | SETIT_010072mg | VII | 0.03 |
| Sorghum | Sorghum_bicolor_NCBIv3 | SORBI_3006G115200 | SORBI_3006G114600 | 6 | 0.04 |
| bicolor | | | | | |
| Triticum | IWGSC | TraesCS2A02G331200 | TraesCS2A02G331800 | 2A | 0.4 |
| aestivum | | | | | |
| Triticum | IWGSC | TraesCS2B02G351000 | TraesCS2B02G350400 | 2B | 0.52 |
| aestivum | | | | | |
| Triticum | IWGSC | TraesCS2D02G331700 | TraesCS2D02G331100 | 2D | 0.16 |
| aestivum | | | | | |
| Triticum | WEWSeq_v.1.0 | TRIDC2BG050920 | TRIDC2BG050840 | 2B | 0.55 |
| dicoccoides | | | | | |
| Triticum | WEWSeq_v.1.0 | TRIDC2AG048440 | TRIDC2AG048380 | 2A | 0.39 |
| dicoccoides | | | | | |
| Eragrostis | CERZOS_E.curvula1.0 | EJB05_39175 | EJB05_39179 | Contig | 0.04 |
| curvula | | | | 30 | |
| Eragrostis tef | ASM97063v1 | Et_s9182-0.2-1.path1 | Et_s9182-0.38-1.path1 | scaffold | 0.05 |
| | | | | 9182 | |
| Panicum hallif | PHallii_v3.1 | PAHAL_7G185900 | PAHAL_7G185400 | 7 | 0.03 |
| FIL2 | | | | | |
| Panicum hallii | PhalliHAL_V2.1 | GQ55_7G177400 | GQ55_7G176900 | 7 | 0.03 |
| HAL2 | | | | | |
| Saccharum | Sspon.HiC_chr_asm | Sspon.05G0009350-1A | Sspon.05G0009380-1A | 5A | 0.02 |
| spontaneum | | | | | |
| Saccharum | Sspon.HiC_chr_asm | Sspon.05G0009350-1P | | 5C | 0.09 |
| spontaneum | | | | | |
| Saccharum | Sspon.HiC_chr_asm | Sspon.05G0009350-4D | Sspon.05G0009380-1T | 5D | 0.05 |
| spontaneum | | | | | |
| Saccharum | Sspon.HiC_chr_asm | Sspon.05G0009350-2B | | 5B | 0.07 |
| spontaneum | | | | | |
| Setaria viridis | Setaria_viridis_v2.0 | SEVIR_7G141200v2 | SEVIR_7G140700v2 | 7 | 0.03 |
| Triticum spelta | PGSBv2.0 | TraesTSP2A01G360100 | TraesTSP2A01G359500 | 2A | 0.37 |
| Triticum spelta | PGSBv2.0 | TraesTSP2B01G384300 | TraesTSP2B01G383700 | 2B | 0.53 |
| Triticum spelta | PGSBv2.0 | TraesTSP2D01G362500 | TraesTSP2D01G361800 | 2D | 0.16 |
| Triticum | Svevo.v1 | TRITD2Av1G202420 | TRITD2Av1G202210 | 2A | 0.41 |
| turgidum | | | | | |
| Triticum | Svevo.v1 | TRITD2Bv1G165690 | TRITD2Bv1G165560 | 2B | 0.52 |
| turgidum | | | | | |
| Secale cereale | RO_Lo7_hq-2018-v1p1_r1- | SECCE2Rv1G0106910.1 | SECCE2Rv1G0106850.1 | chr2R | 0.27 |
| | all_cdna.fasta | | | | |
| Urochloa fusca | v1.1 | | | Chr07 | 0.03 |
| Thinopyrum | v2.1 | | | Chr06 | 0.41 |
| intermedium | | | | | |
| Thinopyrum | v2.1 | | | scaffold_291 | 0.18 |
| intermedium | | | | | |
| Pharus | v1.1 | | | Chr06 | 0.06 |
| latifolius | | | | | |
| Paspalum | v3.1 | | | Chr06 | 0.02 |
| vaginatum | | | | | |
| Panicum | v5.1 | | | Chr07N | 0.08 |
| virgatum | | | | | |
| Panicum | v5.1 | | | Chr07K | 0.04 |
| virgatum | | | | | |
| Oropetium | v1.0 | | | Oropetium_{—} | 0.02 |
| thomaeum | | | | genomic_{—} | |
| | | | | 20141112_091 | |
| Miscanthus | v7.1 | | | Chr11 | 0.53 |
| sinensis | | | | | |
| Eleusine | v1.1 | | | 4A | 0.026 |
| coracana | | | | | |
| Eleusine | v1.1 | | | 4B | 0.051 |
| coracana | | | | | |
| Chasmanthium | v1.1 | | | Chr06 | 0.08 |
| laxum | | | | | |
| Brachypodium | v1.1 | | | chr9 | 0.078 |
| sylvaticum | | | | | |
| Brachypodium | v1.1 | | | Chr09 | 0.04 |
| stacei | | | | | |
| Brachypodium | v1.1 | | | Bm9P | 0.26 |
| mexicanum | | | | | |
| Brachypodium | v1.1 | | | Bm9U | 0.18 |
| mexicanum | | | | | |
| Lolium perenne | | | | Lp_chr2_0 | 0.49 |
| Phyllostachys | | | | hic_scaffold_24 | 0.12 |
| edulis | | | | | |
| Oryza alta | | | | GWHAZTO00000004 | 0.05 |
| Cenchrus | | | | GWHAORA00000014 | 0.11 |
| purpureus | | | | | |
| Digitaria exilis | | | | Dexi_CM05836_chr07A | 0.03 |

Illustrative (partial) cDNA and genomic sequences of Myb80 and Dwarf11 genes of several cereal species are provided in Table 2. Other representative genomic, cDNA, coding, and protein sequences of Myb80 and Dwarf11 genes in other species are provided in SEQ ID NOs: 115-392. (see also Table 3)

These sequences may serve as templates for identifying Myb80 and Dward11 genes in other species.

TABLE 2

| | | SEQ |
|---|---|---|
| Species | Sequence | ID NO |
| Oryza | > Myb80 cDNA | 1 |
| sativa | ATGGGGGGGGTGCCGTGCTGCGAGAAGGAC | |
| | AACGTGAAGCGCGGGCAGTGGACGCCCGAG | |
| | GAGGACAACAAGCTGCTCTCCTACATCACC | |
| | CAGTACGGCACCCGCAACTGGCGCCTCATC | |
| | CCCAAGAACGCCGGGTTGCAGCGGTGCGGG | |
| | AAGAGCTGCCGGCTGCGGTGGACCAACTAC | |
| | CTCCGGCCCGACCTCAAGCACGGCGAGTTC | |
| | ACCGACGCCGAGGAGCAGACCATCATCAAG | |
| | CTCCACTCCGTCGTCGGCAACAGGTGGTCG | |
| | GTGATCGCGGCGCAGCTTCCGGGGCGGACG | |
| | GACAACGACGTGAAGAACCACTGGAACACG | |
| | AAGCTGAAGAAGAAGCTGTCCGGGATGGGC | |
| | ATCGACCCCGTCACGCACAAGTCCTTCTCG | |
| | CACCTCATGGCCGAGATCGCCACCACGCTG | |
| | GCGCCGCCGCAGGTGGCGCACCTCGCCGAG | |
| | GCCGCGCTGGGGTGCTTCAAGGACGAGATG | |
| | CTCCACCTCCTCACCAAGAAGCGCCCCTCC | |
| | GACTTCCCCTCGCCCGCCGTGCACGACGGC | |
| | GCCGGCGCCGGCGCCAGCGCGTCCGCGCTC | |
| | GCCGCGCCCTGTTTCCCCGCCGCGCCGCCG | |
| | CACCACCCGCAGGCCGACGACACCATCGAG | |
| | CGCATCAAGCTCGGCCTGTCCCGCGCCATC | |
| | ATGAGCGATCCCTCCACCGCCTCCGCCGCC | |
| | GCCGCCGCCGCCGCGCCCTCCGCCCCCGCG | |
| | GAGGACAAGCCGTGGCCGCCCGGCGACATG | |
| | TCCGAGGGGCTCGCCGGGATGTACGCCACG | |
| | TACAACCCGGCGGCGCACGCGCACGCGCAG | |
| | GCCCAGGCCGAGTTCCGGTACGACGGGGCC | |
| | TCCGCGGCGCAGGGCTACGTCCTCGGCGGC | |
| | GACGGCGACCAGGGCACGTCGATGTGGAGC | |
| | CACCAGAGCCTGTACAGCGGGAGCTCCGGC | |
| | ACCGAGGAGGCCAGGCGGGAGTTGCCGGAG | |
| | AAGGGCAACGACAGCGTCGGCAGCAGCGGC | |
| | GGCGACGACGACGCCGCGGACGACGGCAAG | |
| | GACAGCGGGAAGGGGGCAGCCTCCGACATG | |
| | TCGGGCCTGTTCGCCTCCGACTGCGTGCTC | |
| | TGGGACTTGCCCGACGAGCTCACGAATCAC | |
| | ATGGTGTAG | |
| Oryza | >Myb80 genomic (LOC_Os04g39470) | 2 |
| sativa | ATTTATAACACGCGGCGCGGCCGTGTGAGC | |
| | GCCGCACGCACGCAGCAGCAGCGGAATCTC | |
| | GACGGCGGCGACATCATGGGGCGGGTGCCG | |
| | TGCTGCGAGAAGGACAACGTGAAGCGCGGG | |
| | CAGTGGACGCCCGAGGAGGACAACAAGCTG | |
| | CTCTCCTACATCACCCAGTACGGCACCCGC | |
| | AACTGGCGCCTCATCCCCAAGAACGCCGGT | |
| | ACGTTGGCGCGCGCGCCGCCACCGGCGAAC | |
| | GCGTGGTTGCAGCAGCGGCGGCGCTCTGAC | |
| | CGGGGTGTTTGTTGCTGGAACGTTGGCAGG | |
| | GTTGCAGCGGTGCGGGAAGAGCTGCCGGCT | |
| | GCGGTGGACCAACTACCTCCGGCCCGACCT | |
| | CAAGCACGGCGAGTTCACCGACGCCGAGGA | |
| | GCAGACCATCATCAAGCTCCACTCCGTCGT | |
| | CGGCAACAGGTAGGCATCAACGAGTGGTCT | |
| | CGCTACACCGTCTTGTGATCTTGGGTCATT | |
| | TTTGGAGGAATGTATTGAGCAATGCGGGAT | |
| | GGGGCTGTGTGTGGCAAGGTGGTCGGTGAT | |
| | CGCGGCGCAGCTTCCGGGGCGGACGGACAA | |
| | CGACGTGAAGAACCACTGGAACACGAAGCT | |
| | GAAGAAGAAGCTGTCCGGGATGGGCATCGA | |
| | CCCCGTCACGCACAAGTCCTTCTCGCACCT | |
| | CATGGCCGAGATCGCCACCACGCTGGCGCC | |
| | GCCGCAGGTGGCGCACCTCGCCGAGGCCGC | |
| | GCTGGGGTGCTTCAAGGACGAGATGCTCCA | |
| | CCTCCTCACCAAGAAGCGCCCCTCCGACTT | |
| | CCCCTCGCCCGCCGTGCACGACGGCGCCGG | |
| | CGCCGGCGCCAGCGCGTCCGCGCTCGCCGC | |
| | GCCCTGTTTCCCCGCCGCGCCGCCGCACCA | |
| | CCCGCAGGCCGACGACACCATCGAGCGCAT | |
| | CAAGCTCGGCCTGTCCCGCGCCATCATGAG | |
| | CGATCCCTCCACCGCCTCCGCCGCCGCCGC | |
| | CGCCGCCGCGCCCTCCGCCCCCGCGGAGGA | |
| | CAAGCCGTGGCCGCCCGGCGACATGTCCGA | |
| | GGGGCTCGCCGGGATGTACGCCACGTACAA | |
| | CCCGGCGGCGCACGCGCACGCGCAGGCCCA | |
| | GGCCGAGTTCCGGTACGACGGGGCCTCCGC | |
| | GGCGCAGGGCTACGTCCTCGGCGGCGACGG | |
| | CGACCAGGGCACGTCGATGTGGAGCCACCA | |
| | GAGCCTGTACAGCGGGAGCTCCGGCACCGA | |
| | GGAGGCCAGGCGGGAGTTGCCGGAGAAGGG | |
| | CAACGACAGCGTCGGCAGCAGCGGCGGCGA | |
| | CGACGACGCCGCGGACGACGGCAAGGACAG | |
| | CGGGAAGGGGGCAGCCTCCGACATGTCGGG | |
| | CCTGTTCGCCTCCGACTGCGTGCTCTGGGA | |
| | CTTGCCCGACGAGCTCACGAATCACATGGT | |
| | GTAGCTAAGCTATAAAACGCGGGAAAACTT | |
| | AGCACGGCGAAGCACGCCATGATCCACATC | |
| | ACTTGCAGTTGCAGGTGGTGAGAATTGGAT | |
| | AGAGCGAAAAATTGCTACCAATAATATTGC | |
| | TGAAAGAGTGACAGAAAAAGTGAAAAACTA | |
| | TAGTATGTAGCTGGCAAGAAAAATGGGGAT | |
| | TCTTTTTGCTTGGAAAAGAGAAAAAAAAGT | |
| | GAGATGTGAATAGTTTTACCGAGAAGCA | |
| Triticum | >Myb80 cDNA | 3 |
| aestivum | CAAGCGCCGCACGCCGATCGAAAACTCAGT | |
| | CGCTCGAACACTCACCCAGCAAGAACGCAG | |
| | CGCCAGCGACGTATCGGCCTACCGGAAAGC | |
| | GGAGCCTCCACGACGGATCCCGTGCGCGCG | |
| | CAGGGCGGTCCGGGAGGCGGAGGCGCCGAC | |
| | ATGGGCCGGATCCCGTGCTGCGAGAAGGAC | |
| | AACGTGAAGCGCGGGCAGTGGACGCCCGAG | |
| | GAGGACAACAAGCTGCTCTCCTACATCACG | |
| | CAGCACGGCACGCGCAACTGGCGCCTCATC | |
| | CCCAAGAACGCCGGGCTGCAGCGATGCGGG | |
| | AAGAGCTGCCGGCTGCGGTGGACCAACTAC | |
| | CTGCGGCCCGACCTCAAGCACGGCGAGTTC | |
| | ACTGACGCCGAGGAGCAGACCATCATCAAG | |
| | CTGCACTCCGTCGTCGGCAACAGGTGGTCG | |
| | GTGATCGCGGCCCAGCTGCCGGGGCGGACG | |
| | GACAACGACGTCAAGAACCACTGGAACACC | |
| | AAGCTCAAGAAGAAGCTGTCCGGGATGGGC | |
| | ATCGACCCCGTCACGCACAAGTCCTTCTCG | |
| | CACCTCATGGCCGAGATCGCCACCACGCTC | |
| | GCCCCGCCGCAGGTGGCGCACCTCGCCGAG | |
| | GCCGCCCTGGGGTGCTTCAAGGACGAGATG | |
| | CTCCACCTCCTCACCAAGAAGCGCCCCACC | |
| | GACTTCCCCTCGCCCGCCATGCCCGACACG | |
| | GCGGCGGGCGCCAGCGCGGGCATGGCCATG | |
| | GGCCCGGGCGCGCACATCGCGCCCTGCTAC | |
| | GCCGCGCCGCCACAAGCCGACGACACCATC | |
| | GAGCGCATCAAGATGGGCCTGTCCCGCGCC | |
| | ATCATGAGCGAGCCCGCCGCTCCCGCGGCC | |
| | GACAAGCCGTGGCCATCGGGCGACATGTCC | |
| | GAGGGGCTGGCCGGCATGTACGCCGCGTTC | |
| | AACCCCGCGCAGGCGACGGAGTTCCGGTAC | |
| | GAAGGGACGGCGTCGGGGTACGTCCTTGGG | |
| | GGCGACGGCGACCAGGGCACGTCGATGTGG | |
| | AGCCACCACAGCATGTACAGCGGGAGCTCC | |
| | GGCACCGAGGGGGGCAGGCCGGCATTGCAG | |
| | GAAAAGGGCAACGACAGTGTCGGCAGCAGC | |
| | GGCGGCGACGAGGAGGCGGAAGACGGCAAG | |
| | GAGGGAGGGAAGGGAGCCTCCGACATGTCG | |
| | GGGCTGTTTGGATCCGATTGCGTACTCTGG | |
| | GACTTGCCCGACGAGCTGACGAATCACATG | |
| | TGTGACTAAATTCACACGGCGAGCTTAGCT | |
| | AGCATCATCGATGAACGTCAGTTCGCCGGA | |
| | GAAAAGAAAGCCTAGAGCCAAACGATTTTA | |
| | GAAGCACATAAATTTCTGACAGTGCCTGAG | |
| | AAGAGCTAGAATAAACAGGGAAACTGGAAA | |
| | ATGGCGTCCCTTCTTCTTTTTTTTCTGCGT | |
| | GCAGAACTGTTAAGTGTACATTGCTCTGCC | |
| | GCTGGCCATGCTATTTTTTGGGGGTCCTGT | |
| | CTACTGTGCTTCAGTCAAAACGACTTCAAT | |
| | TCAAACAGATTTGGTAGCATGTCAATGAAG | |
| | CTGTCTGACCGTAACATGTCAATGTTTTAG | |
| | TAAAACCGCAC | |
| Oryza | >Dwarf11 cDNA (LOC_Os04g39430) | 4 |
| sativa | ATGGTGGGAGGAGAGCTTGTGCTGGCTGCT | |
| | CTGGTGATCCTGCTTGCTTTGCTGCTGACC | |
| | CTGGTGCTGAGCCACTTCCTGCCTTTGCTC | |
| | CTGAATCCCAAGGCTCCCAAGGGAAGCTTT | |
| | GGGTGGCCTCTCCTTGGTGAGACGCTGAGG | |
| | TTCCTCAGTCCTCATGCTAGCAACACCCTG | |
| | GGCAGCTTCCTGGAGGATCACTGCTCCAGG | |
| | TATGGGAGGGTGTTTAAGTCCCATCTGTTC | |
| | TGCACCCCCACCATAGTGTCCTGTGACCAG | |
| | GAGCTGAACCACTTCATCCTTCAGAATGAG | |
| | GAGAGGCTGTTTCAGTGCAGCTACCCCAGG | |
| | CCAATTCATGGCATTCTGGGCAAGTCCTCC | |
| | ATGTTAGTGGTCCTAGGGGAGGACCACAAG | |
| | AGGCTCAGGAACCTTGCTCTAGCACTGGTC | |
| | ACCTCCACAAAGCTCAAGCCCAGCTACCTT | |
| | GGCGACATTGAGAAGATTGCACTGCATATA | |
| | GTTGGGTCATGGCATGGCAAGAGCAAGGAC | |
| | AAGGGGATGGTCAATGTCATCGCCTTCTGC | |
| | GAGGAGGCAAGAAAGTTTGCATTCAGTGTA | |
| | ATAGTGAAGCAGGTGCTGGGGCTATCACCA | |
| | GAGGAGCCGGTCACTGCCATGATACTTGAA | |
| | GATTTCCTCGCCTTCATGAAGGGTCTCATC | |
| | TCTTTCCCTCTCTACATCCCAGGGACGCCC | |
| | TATGCCAAAGCTGTGCAGGCCAGAGCGAGG | |
| | ATATCAAGCACTGTGAAGGGTATTATTGAG | |
| | GAGAGGAGGAATGCTGGCTCCAGCAACAAG | |
| | GGTGATTTCCTTGATGTGCTGCTTTCAAGC | |
| | AATGAGCTCTCTGATGAGGAGAAAGTGAGC | |
| | TTTGTGCTGGATTCCTTACTGGGAGGATAT | |
| | GAGACCACCTCACTCTTGATCTCCATGGTT | |
| | GTGTATTTCCTTGGGCAGTCAGCTCAAGAT | |
| | CTGGAACTAGTGAAGAGGGAGCATGAAGGC | |
| | ATAAGATCGAAGAAAGAGAAGGACGAGTTC | |
| | TTGAGCTCTGAAGACTATAAGAAGATGGAA | |
| | TATACCCAACATGTAAGATGA | |

TABLE 3

| | | SEQ |
|---|---|---|
| Species | Sequence | ID NO |
| Aegilops | Dwarf11 cDNA sequence - AET2Gv20748100.1 | 115 |
| tauschii | Dwarf11 coding sequence - AET2Gv20748100.1 | 116 |
| | Dwarf11 protein sequence - AET2Gv20748100.1 | 117 |
| | Dwarf11 cDNA sequence - AET2Gv20748100.9 | 118 |
| | Dwarf11 coding sequence - AET2Gv20748100.9 | 119 |
| | Dwarf11 protein sequence - AET2Gv20748100.9 | 120 |
| | Dwarf11 cDNA sequence - AET2Gv20748100.2 | 121 |
| | Dwarf11 coding sequence - AET2Gv20748100.2 | 122 |
| | Dwarf11 protein sequence - AET2Gv20748100.2 | 123 |
| | Dwarf11 cDNA sequence - AET2Gv20748100.7 | 124 |
| | Dwarf11 coding sequence - AET2Gv20748100.7 | 125 |
| | Dwarf11 protein sequence - AET2Gv20748100.7 | 126 |
| | Dwarf11 cDNA sequence - AET2Gv20748100.10 | 127 |
| | Dwarf11 coding sequence - AET2Gv20748100.10 | 128 |
| | Dwarf11 protein sequence - AET2Gv20748100.10 | 129 |
| | Dwarf11 cDNA sequence - AET2Gv20748100.8 | 130 |
| | Dwarf11 coding sequence - AET2Gv20748100.8 | 131 |
| | Dwarf11 protein sequence - AET2Gv20748100.8 | 132 |
| | Dwarf11 cDNA sequence - AET2Gv20748100.4 | 133 |
| | Dwarf11 coding sequence - AET2Gv20748100.4 | 134 |
| | Dwarf11 protein sequence - AET2Gv20748100.4 | 135 |
| | Dwarf11 cDNA sequence - AET2Gv20748100.5 | 136 |
| | Dwarf11 coding sequence - AET2Gv20748100.5 | 137 |
| | Dwarf11 protein sequence - AET2Gv20748100.5 | 138 |
| | Dwarf11 cDNA sequence - AET2Gv20748100.12 | 139 |
| | Dwarf11 coding sequence - AET2Gv20748100.12 | 140 |
| | Dwarf11 protein sequence - AET2Gv20748100.12 | 141 |
| | Dwarf11 cDNA sequence - AET2Gv20748100.11 | 142 |
| | Dwarf11 coding sequence - AET2Gv20748100.11 | 143 |
| | Dwarf11 protein sequence - AET2Gv20748100.11 | 144 |
| | Dwarf11 cDNA sequence - AET2Gv20748100.3 | 145 |
| | Dwarf11 coding sequence - AET2Gv20748100.3 | 146 |
| | Dwarf11 protein sequence - AET2Gv20748100.3 | 147 |
| | Dwarf11 cDNA sequence - AET2Gv20748100.6 | 148 |
| | Dwarf11 coding sequence - AET2Gv20748100.6 | 149 |
| | Dwarf11 protein sequence - AET2Gv20748100.6 | 150 |
| | Dwarf11 genomic sequence - | 151 |
| | Aet_v4.0:2D:422816950:422822140:1 | |
| Aegilops | Myb80 cDNA sequence - AET2Gv20749000.1 | 152 |
| tauschii | Myb80 coding sequence - AET2Gv20749000.1 | 153 |
| | Myb80 protein sequence - AET2Gv20749000.1 | 154 |
| | Myb80 cDNA sequence - AET2Gv20749000.2 | 155 |
| | Myb80 coding sequence - AET2Gv20749000.2 | 156 |
| | Myb80 protein sequence - AET2Gv20749000.2 | 157 |
| | Myb80 cDNA sequence - AET2Gv20749000.3 | 158 |
| | Myb80 coding sequence - AET2Gv20749000.3 | 159 |
| | Myb80 protein sequence - AET2Gv20749000.3 | 160 |
| | Myb80 genomic sequence - | 161 |
| | Aet_v4.0:2D:422984632:422987728:−1 | |
| Brachypodium | Dwarf11 cDNA sequence - KQJ83091 | 162 |
| distachyon | Dwarf11 protein sequence - KQJ83091 | 163 |
| | Dwarf11 genomic sequence - | 164 |
| | Brachypodium_distachyon_v3.0:5:16687949:16694228:−1 | |
| Brachypodium | Myb80 cDNA sequence - KQJ83102 | 165 |
| distachyon | Myb80 coding sequence - KQJ83102 | 166 |
| | Myb80 protein sequence - KQJ83102 | 167 |
| | Myb80 genomic sequence - | 168 |
| | Brachypodium_distachyon_v3.0:5:16741184:16744157:−1 | |
| Hordeum | Dwarf11 cDNA sequence | 169 |
| vulgare | Dwarf11 protein sequence | 170 |
| | Dwarf11 genomic sequence - | 171 |
| | HOVUSG0946300_genomic_scaffold67:5620836-5626353 | |
| Hordeum | Myb80 cDNA sequence | 172 |
| vulgare | Myb80 protein sequence | 173 |
| | Myb80 genomic sequence - | 174 |
| | HOVUSG0945200_genomic_scaffold67:4914185-4916639 | |
| Leersia perrieri | Dwarf11 cDNA sequence - LPERR04G12330.1 | 175 |
| | Dwarf11 coding sequence - LPERR04G12330.1 | 176 |
| | Dwarf11 protein sequence - LPERR04G12330.1 | 177 |
| | Dwarf11 cDNA sequence - LPERR04G12330.2 | 178 |
| | Dwarf11 coding sequence - LPERR04G12330.2 | 179 |
| | Dwarf11 protein sequence - LPERR04G12330.2 | 180 |
| | Dwarf11 genomic sequence - | 181 |
| | Lperr_V1.4:4:13356928:13362832:−1 | |
| Leersia perrieri | Myb80 cDNA sequence - LPERR04G12420.1 | 182 |
| | Myb80 coding sequence - LPERR04G12420.1 | 183 |
| | Myb80 protein sequence - LPERR04G12420.1 | 184 |
| | Myb80 genomic sequence - | 185 |
| | Lperr_V1.4:4:13405809:13408196:−1 | |
| Oryza barthii | Dwarf11 cDNA sequence - OBART04G15600.1 | 186 |
| | Dwarf11 coding sequence - OBART04G15600.1 | 187 |
| | Dwarf11 protein sequence - OBART04G15600.1 | 188 |
| | Dwarf11 genomic sequence - | 189 |
| | O.barthii_v1:4:16843786:16848824:−1 | |
| Oryza | Dwarf11 cDNA sequence - OB04G22900.1 | 190 |
| brachyantha | Dwarf11 coding sequence - OB04G22900.1 | 191 |
| | Dwarf11 protein sequence - OB04G22900.1 | 192 |
| | Dwarf11 genomic sequence - | 193 |
| | Oryza_brachyantha.v1.4b:4:11837902:11843774:−1 | |
| Oryza | Myb80 cDNA sequence - OB04G23000.1 | 194 |
| brachyantha | Myb80 coding sequence - OB04G23000.1 | 195 |
| | Myb80 protein sequence - OB04G23000.1 | 196 |
| | Myb80 genomic sequence - | 197 |
| | Oryza_brachyantha.v1.4b:4:11905173:11908285:−1 | |
| Oryza | Dwarf11 cDNA sequence - ORGLA04G0126200.1 | 198 |
| glaberrima | Dwarf11 coding sequence - ORGLA04G0126200.1 | 199 |
| | Dwarf11 protein sequence - ORGLA04G0126200.1 | 200 |
| | Dwarf11 genomic sequence - | 201 |
| | Oryza_glaberrima_V1:4:15903746:15908791:−1 | |
| Oryza | Myb80 cDNA sequence - ORGLA04G0126700.1 | 202 |
| glaberrima | Myb80 coding sequence - ORGLA04G0126700.1 | 203 |
| | Myb80 protein sequence - ORGLA04G0126700.1 | 204 |
| | Myb80 genomic sequence - | 205 |
| | Oryza_glaberrima_V1:4:15945446:15947954:−1 | |
| Oryza | Dwarf11 cDNA sequence - OGLUM04G15480.1 | 206 |
| glumipatula | Dwarf11 coding sequence - OGLUM04G15480.1 | 207 |
| | Dwarf11 protein sequence - OGLUM04G15480.1 | 208 |
| | Dwarf11 genomic sequence - | 209 |
| | Oryza_glumaepatula_v1.5:4:20865448:20870495:−1 | |
| Oryza | Myb80 cDNA sequence - OGLUM04G15520.1 | 210 |
| glumipatula | Myb80 coding sequence - OGLUM04G15520.1 | 211 |
| | Myb80 protein sequence - OGLUM04G15520.1 | 212 |
| | Myb80 genomic sequence - | 213 |
| | Oryza_glumaepatula_v1.5:4:20901294:20903799:−1 | |
| Oryza indica | Dwarf11 cDNA sequence - GIOSGA014915-TA | 214 |
| | Dwarf11 coding sequence - GIOSGA014915-TA | 215 |
| | Dwarf11 protein sequence - GIOSGA014915-TA | 216 |
| | Dwarf11 genomic sequence - | 217 |
| | ASM465v1:4:21874459:21879503:−1 | |
| Oryza indica | Myb80 cDNA sequence - BGIOSGA030830-TA | 218 |
| | Myb80 coding sequence - BGIOSGA030830-TA | 219 |
| | Myb80 protein sequence - BGIOSGA030830-TA | 220 |
| | Myb80 genomic sequence - | 221 |
| | ASM465v1:9:14727035:14731412:1 | |
| Oryza indica | Myb80 cDNA sequence - BGIOSGA014912-TA | 222 |
| | Myb80 coding sequence - BGIOSGA014912-TA | 223 |
| | Myb80 protein sequence - BGIOSGA014912-TA | 224 |
| | Myb80 genomic sequence - | 225 |
| | ASM465v1:4:21914917:21917416:−1 | |
| Oryza | Dwarf11 cDNA sequence - KN538714.1_FGT040 | 226 |
| longistaminata | Dwarf11 coding sequence - KN538714.1_FGT040 | 227 |
| | Dwarf11 protein sequence - KN538714.1_FGT040 | 228 |
| | Dwarf11 genomic sequence - | 229 |
| | O_longistaminata_v1.0:KN538714.1:251095:256161:−1 | |
| Oryza | Myb80 cDNA sequence - KN538714.1_FGT046 | 230 |
| longistaminata | Myb80 coding sequence - KN538714.1_FGT046 | 231 |
| | Myb80 protein sequence - KN538714.1_FGT046 | 232 |
| | Myb80 genomic sequence - | 233 |
| | O_longistaminata_v1.0:KN538714.1:278750:289881:−1 | |
| Oryza | Dwarf11 cDNA sequence - OMERI04G13440.1 | 234 |
| meridionalis | Dwarf11 coding sequence - OMERI04G13440.1 | 235 |
| | Dwarf11 protein sequence - OMERI04G13440.1 | 236 |
| | Dwarf11 genomic sequence - | 237 |
| | ryza_meridionalis_v1.3:4:18984801:18990529:−1 | |
| Oryza | Myb80 cDNA sequence - OMERI04G13500.1 | 238 |
| meridionalis | Myb80 coding sequence - OMERI04G13500.1 | 239 |
| | Myb80 protein sequence - OMERI04G13500.1 | 240 |
| | Myb80 cDNA sequence - OMERI04G13500.2 | 241 |
| | Myb80 coding sequence - OMERI04G13500.2 | 242 |
| | Myb80 protein sequence - OMERI04G13500.2 | 243 |
| | Myb80 cDNA sequence - OMERI04G13500.3 | 244 |
| | Myb80 coding sequence - OMERI04G13500.3 | 245 |
| | Myb80 protein sequence - OMERI04G13500.3 | 246 |
| | Myb80 cDNA sequence - OMERI04G13500.4 | 247 |
| | Myb80 coding sequence - OMERI04G13500.4 | 248 |
| | Myb80 protein sequence - OMERI04G13500.4 | 249 |
| | Myb80 cDNA sequence - OMERI04G13500.5 | 250 |
| | Myb80 coding sequence - OMERI04G13500.5 | 251 |
| | Myb80 protein sequence - OMERI04G13500.5 | 252 |
| | Myb80 genomic sequence - | 253 |
| | Oryza_meridionalis_v1.3:4:19037892:19050475:−1 | |
| Oryza nivara | Dwarf11 cDNA sequence - ONIVA04G13750.1 | 254 |
| | Dwarf11 coding sequence - ONIVA04G13750.1 | 255 |
| | Dwarf11 protein sequence - ONIVA04G13750.1 | 256 |
| | Dwarf11 genomic sequence - | 257 |
| | Oryza_nivara_v1.0:4:16129366:16134411:−1 | |
| Oryza nivara | Myb80 cDNA sequence - ONIVA04G13800.1 | 258 |
| | Myb80 coding sequence - ONIVA04G13800.1 | 259 |
| | Myb80 protein sequence - ONIVA04G13800.1 | 260 |
| | Myb80 genomic sequence - | 261 |
| | Oryza_nivara_v1.0:4:16169880:16172391:−1 | |
| Oryza punctata | Dwarf11 cDNA sequence - OPUNC04G13510.1 | 262 |
| | Dwarf11 coding sequence - OPUNC04G13510.1 | 263 |
| | Dwarf11 protein sequence - OPUNC04G13510.1 | 264 |
| | Dwarf11 genomic sequence - | 265 |
| | Oryza_punctata_v1.2:4:21171295:21176328:−1 | |
| Oryza punctata | Myb80 cDNA sequence - OPUNC04G13560.1 | 266 |
| | Myb80 coding sequence - OPUNC04G13560.1 | 267 |
| | Myb80 protein sequence - OPUNC04G13560.1 | 268 |
| | Myb80 genomic sequence - | 269 |
| | Oryza_punctata_v1.2:4:21205485:21207976:−1 | |
| Oryza | Dwarf11 cDNA sequence - ORUFI04G16950.1 | 270 |
| rufipogon | Dwarf11 coding sequence - ORUFI04G16950.1 | 271 |
| | Dwarf11 protein sequence - ORUFI04G16950.1 | 272 |
| | Dwarf11 genomic sequence - | 273 |
| | OR_W1943:4:19480753:19485797:−1 | |
| Oryza | Myb80 cDNA sequence - ORUFI04G17000.1 | 274 |
| rufipogon | Myb80 coding sequence - ORUFI04G17000.1 | 275 |
| | Myb80 protein sequence - ORUFI04G17000.1 | 276 |
| | Myb80 genomic sequence - | 277 |
| | OR_W1943:4:19522579:19525090:−1 | |
| Oryza sativa | Dwarf11 cDNA sequence - Os04t0469800-01 | 278 |
| | Dwarf11 coding sequence - Os04t0469800-01 | 279 |
| | Dwarf11 protein sequence - Os04t0469800-01 | 280 |
| | Dwarf11 cDNA sequence - Os04t0469800-02 | 281 |
| | Dwarf11 coding sequence - Os04t0469800-02 | 282 |
| | Dwarf11 protein sequence - Os04t0469800-02 | 283 |
| | Dwarf11 genomic sequence - IRGSP- | 284 |
| | 1.0:4:23466567:23472192:−1 | |
| Oryza sativa | Myb80 cDNA sequence - Os04t0470600-00 | 285 |
| | Myb80 coding sequence - Os04t0470600-00 | 286 |
| | Myb80 protein sequence - Os04t0470600-00 | 287 |
| | Myb80 genomic sequence - IRGSP- | 288 |
| | 1.0:4:23510635:23512134:−1 | |
| Setaria italica | Dwarf11 cDNA sequence - KQK97661 | 289 |
| | Dwarf11 coding sequence - KQK97661 | 290 |
| | Dwarf11 protein sequence - KQK97661 | 291 |
| | Dwarf11 genomic sequence - | 292 |
| | Setaria_italica_v2.0:VII:22472930:22477595:−1 | |
| Setaria italica | Myb80 cDNA sequence - KQK97666 | 293 |
| | Myb80 coding sequence - KQK97666 | 294 |
| | Myb80 protein sequence - KQK97666 | 295 |
| | Myb80 genomic sequence - | 296 |
| | Setaria_italica_v2.0:VII:22506182:22508488:−1 | |
| Sorghum | Dwarf11 cDNA sequence - EES10969 | 297 |
| bicolor | Dwarf11 coding sequence - EES10969 | 298 |
| | Dwarf11 protein sequence - EES10969 | 299 |
| | Dwarf11 genomic sequence - | 300 |
| | Sorghum_bicolor_NCBIv3:6:48244995:48250831:1 | |
| Sorghum | Myb80 cDNA sequence - EES12341 | 301 |
| bicolor | Myb80 coding sequence - EES12341 | 302 |
| | Myb80 protein sequence - EES12341 | 303 |
| | Myb80 genomic sequence - | 304 |
| | Sorghum_bicolor_NCBIv3:6:48285459:48288496:−1 | |
| Triticum | Dwarf11-A cDNA sequence - TraesCS2A02G331800.2 | 305 |
| aestivum | Dwarf11-A coding sequence - TraesCS2A02G331800.2 | 306 |
| | Dwarf11-A protein sequence - TraesCS2A02G331800.2 | 307 |
| | Dwarf11-A cDNA sequence - TraesCS2A02G331800.1 | 308 |
| | Dwarf11-A coding sequence - TraesCS2A02G331800.1 | 309 |
| | Dwarf11-A protein sequence - TraesCS2A02G331800.1 | 310 |
| | Dwarf11-A genomic sequence - | 311 |
| | IWGSC:2A:564616207:564621455:−1 | |
| Triticum | Dwarf11-B cDNA sequence - TraesCS2B02G350400.2 | 312 |
| aestivum | Dwarf11-B coding sequence - TraesCS2B02G350400.2 | 313 |
| | Dwarf11-B protein sequence - TraesCS2B02G350400.2 | 314 |
| | Dwarf11-B cDNA sequence - TraesCS2B02G350400.1 | 315 |
| | Dwarf11-B coding sequence - TraesCS2B02G350400.1 | 316 |
| | Dwarf11-B protein sequence - TraesCS2B02G350400.1 | 317 |
| | Dwarf11-B genomic sequence - | 318 |
| | IWGSC:2B:497794914:497800257:1 | |
| Triticum | Dwarf11-D cDNA sequence - TraesCS2D02G331100.1 | 319 |
| aestivum | Dwarf11-D coding sequence - TraesCS2D02G331100.1 | 320 |
| | Dwarf11-D protein sequence - TraesCS2D02G331100.1 | 321 |
| | Dwarf11-D genomic sequence - | 322 |
| | IWGSC:2D:423852701:423857987:1 | |
| Triticum | Myb80-A cDNA sequence - TraesCS2A02G331200.1 | 323 |
| aestivum | Myb80-A coding sequence - TraesCS2A02G331200.1 | 324 |
| | Myb80-A protein sequence - TraesCS2A02G331200.1 | 325 |
| | Myb80-A genomic sequence - | 326 |
| | IWGSC:2A:564217648:564220489:1 | |
| Triticum | Myb80-B cDNA sequence - TraesCS2B02G351000.1 | 327 |
| aestivum | Myb80-B coding sequence - TraesCS2B02G351000.1 | 328 |
| | Myb80-B protein sequence - TraesCS2B02G351000.1 | 329 |
| | Myb80-B genomic sequence - | 330 |
| | IWGSC:2B:498308950:498311765:−1 | |
| Triticum | Myb80-D cDNA sequence - TraesCS2D02G331700.1 | 331 |
| aestivum | Myb80-D coding sequence - TraesCS2D02G331700.1 | 332 |
| | Myb80-D protein sequence - TraesCS2D02G331700.1 | 333 |
| | Myb80-D genomic sequence - | 334 |
| | IWGSC:2D:424013456:424016258:−1 | |
| Triticum | Dwarf11-A cDNA sequence - TRIDC2AG048380.8 | 335 |
| dicoccoides | Dwarf11-A coding sequence - TRIDC2AG048380.8 | 336 |
| | Dwarf11-A protein sequence - TRIDC2AG048380.8 | 337 |
| | Dwarf11-A cDNA sequence - TRIDC2AG048380.2 | 338 |
| | Dwarf11-A coding sequence - TRIDC2AG048380.2 | 339 |
| | Dwarf11-A protein sequence - TRIDC2AG048380.2 | 340 |
| | Dwarf11-A cDNA sequence - TRIDC2AG048380.5 | 341 |
| | Dwarf11-A coding sequence - TRIDC2AG048380.5 | 342 |
| | Dwarf11-A protein sequence - TRIDC2AG048380.5 | 343 |
| | Dwarf11-A cDNA sequence - TRIDC2AG048380.6 | 344 |
| | Dwarf11-A coding sequence - TRIDC2AG048380.6 | 345 |
| | Dwarf11-A protein sequence - TRIDC2AG048380.6 | 346 |
| | Dwarf11-A cDNA sequence - TRIDC2AG048380.3 | 347 |
| | Dwarf11-A coding sequence - TRIDC2AG048380.3 | 348 |
| | Dwarf11-A protein sequence - TRIDC2AG048380.3 | 349 |
| | Dwarf11-A cDNA sequence - TRIDC2AG048380.4 | 350 |
| | Dwarf11-A coding sequence - TRIDC2AG048380.4 | 351 |
| | Dwarf11-A protein sequence - TRIDC2AG048380.4 | 352 |
| | Dwarf11-A cDNA sequence - TRIDC2AG048380.7 | 353 |
| | Dwarf11-A coding sequence - TRIDC2AG048380.7 | 354 |
| | Dwarf11-A protein sequence - TRIDC2AG048380.7 | 355 |
| | Dwarf11-A cDNA sequence - TRIDC2AG048380.1 | 356 |
| | Dwarf11-A coding sequence - TRIDC2AG048380.1 | 357 |
| | Dwarf11-A protein sequence - TRIDC2AG048380.1 | 358 |
| | Dwarf11-A genomic sequence - | 359 |
| | WEWSeq_v.1.0:2A:561794653:561799882:1 | |
| Triticum | Dwarf11-B cDNA sequence - TRIDC2BG050840.1 | 360 |
| dicoccoides | Dwarf11-B coding sequence - TRIDC2BG050840.1 | 361 |
| | Dwarf11-B protein sequence - TRIDC2BG050840.1 | 362 |
| | Dwarf11-B cDNA sequence - TRIDC2BG050840.2 | 363 |
| | Dwarf11-B coding sequence - TRIDC2BG050840.2 | 364 |
| | Dwarf11-B protein sequence - TRIDC2BG050840.2 | 365 |
| | Dwarf11-B cDNA sequence - TRIDC2BG050840.3 | 366 |
| | Dwarf11-B coding sequence - TRIDC2BG050840.3 | 367 |
| | Dwarf11-B protein sequence - TRIDC2BG050840.3 | 368 |
| | Dwarf11-B cDNA sequence - TRIDC2BG050840.4 | 369 |
| | Dwarf11-B coding sequence - TRIDC2BG050840.4 | 370 |
| | Dwarf11-B protein sequence - TRIDC2BG050840.4 | 371 |
| | Dwarf11-B genomic sequence - | 372 |
| | WEWSeq_v.1.0:2B:496937309:496942545:1 | |
| Triticum | Myb80-B cDNA sequence - TRIDC2BG050920.1 | 373 |
| dicoccoides | Myb80-B coding sequence - TRIDC2BG050920.1 | 374 |
| | Myb80-B protein sequence - TRIDC2BG050920.1 | 375 |
| | Myb80-B genomic sequence - | 376 |
| | WEWSeq_v.1.0:2B:497491088:497492336:−1 | |
| Triticum urartu | Dwarf11 cDNA sequence - TRIUR3_23488-T1 | 377 |
| | Dwarf11 coding sequence - TRIUR3_23488-T1 | 378 |
| | Dwarf11 protein sequence - TRIUR3_23488-T1 | 379 |
| | Dwarf11 genomic sequence - | 380 |
| | ASM34745v1:scaffold51589:70981:76074:1 | |
| Triticum urartu | Myb80 cDNA sequence - TRIUR3_35325-T1 | 381 |
| | Myb80 coding sequence - TRIUR3_35325-T1 | 382 |
| | Myb80 protein sequence - TRIUR3_35325-T1 | 383 |
| | Myb80 genomic sequence - | 384 |
| | ASM34745v1:scaffold23868:10710:13155:−1 | |
| Zea mays | Dwarf11 cDNA sequence - Zm00001d003349_T001 | 385 |
| | Dwarf11 coding sequence - Zm00001d003349_T001 | 386 |
| | Dwarf11 protein sequence - Zm00001d003349_T001 | 387 |
| | Dwarf11 genomic sequence - | 388 |
| | B73_RefGen_v4:2:41027815:41032513:−1 | |
| Zea mays | Myb80 cDNA sequence - Zm00001d025664_T001 | 389 |
| | Myb80 coding sequence - Zm00001d025664_T001 | 390 |
| | Myb80 protein sequence - Zm00001d025664_T001 | 391 |
| | Myb80 genomic sequence - | 392 |
| | B73_RefGen_v4:10:125369198:125372071:−1 | |

When combining mutations in both genes, plants can be obtained which are fertile in the heterozygous stage showing a normal growth phenotype. Seeds produced from these plants via selfing show in 25% of the seeds a small grain phenotype. These small grain seeds result in sterile plants. Therefore, the sterile plants necessary for hybrid seed production can be easily identified from a pool of seeds produced from selfed heterozygous plants.

The general principle of finding specific gene pairs in the genome of a plant species may be applied to get a combination of male sterility and a morphological trait in all plant species including dicot species. So the system can equally be applied to crop species where no hybrid system is even foreseeable at the moment.

### Generation of Individual Knock Out Mutants in Barley Plants

Guide RNAs (gRNAs) targeting the first exon of either the Myb80 or the Dwarf11 gene are developed. Illustrative (Cpf1) gRNAs for the Hordeum vulgare Dwarf 11 (targeting exon 1) are provided in Table 4 (in RNA T is to be replaced by U). Transgenic barley plants are produced containing the respective Cpf1 constructs. Alternatively, it is also possible to provide the Cpf1 construct in a transient way, so no stable integration occurs. This can be achieved by particle bombardment of plasmid DNA without applying selection pressure or by providing RNP's.

TABLE 4

| | | SEQ |
|---|---|---|
| | | ID |
| Identifier | gRNA sequence | NO |
| HvDWARF11-1 | tttgctcctcaaccccaaggctccgagg | 5 |
| HvDWARF11-2 | tttggatggccccttgttggtgagaccc | 6 |
| HvDWARF11-3 | tttgtggaggtgaccagggccagagcaa | 7 |
| HvDWARF11-4 | tttcttgcctcctcgcagaaggtgatgt | 8 |
| HvDWARF11-5 | tttgcattcagtgtgatagtgaagcagg | 9 |
| HvDWARF11-6 | tttcctctccgcatcccagggaccccct | 10 |
| HvDWARF11-7 | tttggcatagggggtccctgggatgcgg | 11 |
| HvDWARF11-8 | tttcctcaacgtgctcctgtcaaccgat | 12 |
| HvDWARF11-9 | tttctcctcgtcagagagttcatcggtt | 13 |
| HvDWARF11-10 | tttgtgctggactccctgctgggaggat | 14 |
| HvDWARF11-11 | tttggtagatcttatgccttgatgctcc | 15 |
| HvDWARF11-12 | tttccagccagatggaatcagatactct | 16 |
| HvDWARF11-13 | tttagtgctgttcatctgaacccctcac | 17 |
| HvDWARF11-14 | tttccatgaagtgaggggttcagatgaa | 18 |
| HvDWARF11-15 | tttcagccttgcagatgggaggtatgtc | 19 |
| HvDWARF11-16 | tttacgcccttcggcggtggcaccaggc | 20 |
| HvDWARF11-17 | tttggcaagctctgatccagggcagagc | 21 |
| HvDWARF11-18 | tttcttcctccatcaccttgtgctgaat | 22 |
| HvDWARF11-19 | tttacctgaaattcagcacaaggtgatg | 23 |
| HvDWARF11-20 | tttggcgccctgcagatggaaaatagat | 24 |
| HvDWARF11-21 | tttcgatcggcaggcctctcgggaactc | 25 |
| HvDWARF11-22 | tccggcgatcctgctcgccttgctcctg | 26 |
| HvDWARF11-23 | ttcctgcctttgctcctcaaccccaagg | 27 |
| HvDWARF11-24 | tccgaggggcagctttggatggcccctt | 28 |
| HvDWARF11-25 | tccaaagctgcccctcggagccttgggg | 29 |
| HvDWARF11-26 | ttcctcacccctcacgcttccaacacgc | 30 |
| HvDWARF11-27 | ttccaacacgctgggtagcttcctggag | 31 |
| HvDWARF11-28 | tccaacacgctgggtagcttcctggagg | 32 |
| HvDWARF11-29 | ttcctggaggatcactgctccaggtatt | 33 |
| HvDWARF11-30 | tccaggaagctacccagcgtgttggaag | 34 |
| HvDWARF11-31 | ttcaagtcccatctgttctgcaccccca | 35 |
| HvDWARF11-32 | tcccatctgttctgcacccccaccgtgg | 36 |
| HvDWARF11-33 | ttcagctcctggtcacaggacaccacgg | 37 |
| HvDWARF11-34 | ttcatcctgcagaatgaggagaggctgt | 38 |
| HvDWARF11-35 | ttccagtgcagctaccccaggccaattc | 39 |
| HvDWARF11-36 | tccagtgcagctaccccaggccaattca | 40 |
| HvDWARF11-37 | ttcatggcatactgggcaagtcctccat | 41 |
| HvDWARF11-38 | tccatgctggtggtcctgggggaggacc | 42 |
| HvDWARF11-39 | tcccccaggaccaccagcatggaggact | 43 |
| HvDWARF11-40 | ttcctgagcctcttgtggtcctccccca | 44 |
| HvDWARF11-41 | tccacaaagctcaagccaagctaccttg | 45 |
| HvDWARF11-42 | tccaactatatgcagcgcaatcctctca | 46 |
| HvDWARF11-43 | ttcagtgtgatagtgaagcaggtgctgg | 47 |
| HvDWARF11-44 | ttcactatcacactgaatgcaaactgcg | 48 |
| HvDWARF11-45 | tccagtatcatggcagtgactggctcct | 49 |
| HvDWARF11-46 | ttcctcaccttcatgaagggcctcatct | 50 |
| HvDWARF11-47 | ttcatgaagggcctcatctcttttcctc | 51 |
| HvDWARF11-48 | ttcatgaaggtgaggaagtcctccagta | 52 |
| HvDWARF11-49 | ttcctcaccttcatgaagggcctcatct | 53 |
| HvDWARF11-50 | ttcatgaagggcctcatctcttttcctc | 54 |
| HvDWARF11-51 | ttcatgaaggtgaggaagtcctccagta | 55 |
| HvDWARF11-52 | ttcctctccgcatcccagggacccccta | 56 |
| HvDWARF11-53 | tcccagggaccccctatgccaaagctgt | 57 |
| HvDWARF11-54 | tccctgggatgcggagaggaaaagagat | 58 |
| HvDWARF11-55 | ttcacagtgcttgatatcctctctctgg | 59 |
| HvDWARF11-56 | ttcctcctctcctcaataatgcccttca | 60 |
| HvDWARF11-57 | tccctcttgcagcagtcagccttcctcc | 61 |
| HvDWARF11-58 | ttcctcaacgtgctcctgtcaaccgatg | 62 |
| HvDWARF11-59 | ttcatcggttgacaggagcacgttgagg | 63 |
| HvDWARF11-60 | tccagcacaaaactcactttctcctcgt | 64 |
| HvDWARF11-61 | tccctgctgggaggatatgagaccacct | 65 |
| HvDWARF11-62 | tcccagcagggagtccagcacaaaactc | 66 |
| HvDWARF11-63 | tccatggtcgtctacttccttgggcagt | 67 |
| HvDWARF11-64 | ttccttgggcagtcagctcaagatctgg | 68 |
| HvDWARF11-65 | tccagatcttgagctgactgcccaagga | 69 |
| HvDWARF11-66 | ttcactaggtccagatcttgagctgact | 70 |
| HvDWARF11-67 | ttcgctttggtagatcttatgccttgat | 71 |
| HvDWARF11-68 | tccatcttcttgtagtcctcagagctca | 72 |
| HvDWARF11-69 | ttccatcttcttgtagtcctcagagctc | 73 |
| HvDWARF11-70 | ttccatctggctggaaagtcctacctgt | 74 |
| HvDWARF11-71 | tccatctggctggaaagtcctacctgtt | 75 |
| HvDWARF11-72 | tccagccagatggaatcagatactctgc | 76 |
| HvDWARF11-73 | ttccagccagatggaatcagatactctg | 77 |
| HvDWARF11-74 | ttcatctgaacccctcacttcatggaaa | 78 |
| HvDWARF11-75 | ttcagatgaacagcactaaaaacaggta | 79 |
| HvDWARF11-76 | ttcatggaaatgcccaacagtttcagcc | 80 |
| HvDWARF11-77 | tccatgaagtgaggggttcagatgaaca | 81 |
| HvDWARF11-78 | ttcagccttgcagatgggaggtatgtcc | 82 |
| HvDWARF11-79 | tcccatctgcaaggctgaaactgttggg | 83 |
| HvDWARF11-80 | ttcggcggtggcaccaggctctgccctg | 84 |
| HvDWARF11-81 | tccagggcagagcctggtgccaccgccg | 85 |
| HvDWARF11-82 | ttcctccatcaccttgtgctgaatttca | 86 |
| HvDWARF11-83 | tccatcaccttgtgctgaatttcaggta | 87 |
| HvDWARF11-84 | ttcagcacaaggtgatggaggaagaaag | 88 |
| HvDWARF11-85 | ttccaatggcatacccgtacgtggagtt | 89 |
| HvDWARF11-86 | tccaatggcatacccgtacgtggagttc | 90 |
| HvDWARF11-87 | tccacgtacgggtatgccattggaatgt | 91 |
| HvDWARF11-88 | ttcccgagaggcctgccgatcgaaatcg | 92 |
| HvDWARF11-89 | tcccgagaggcctgccgatcgaaatcga | 93 |
| HvDWARF11-90 | ttcgatcggcaggcctctcgggaactcc | 94 |
| HvDWARF11-91 | ttcagagcaaattggctcgatttcgatc | 95 |
| HvMyb80-1 | TTTCCTGCAATGCCGGCCTGCCCCCCTc | 96 |
| HvMyb80-2 | TTTGGATCCGATTGCGTACTCTGGGACt | 97 |
| HvMyb80-3 | TTCACGTTGTCCTTCTCGCAGCAAGGGa | 98 |
| HvMyb80-4 | TTCCTCGGGCGTCCACTGCCCGCGCTTC | 99 |
| HvMyb80-5 | TTCCCGCAGCGCTGCAGCCCGGCGTTCt | 100 |
| HvMyb80-6 | TTCACTGACGCCGAGGAGCAGACCATCa | 101 |
| HvMyb80-7 | TTCCAGTGGTTCTTGACGTCGTTGTCCg | 102 |
| HvMyb80-8 | TTCAAGGACGAGATGCTCCACCTCCTCa | 103 |
| HvMyb80-9 | TTCCCCTCGCCCGCCATGCCCGACATGg | 104 |
| HvMyb80-10 | TTCGCCGCGCCGCCACAAGCCGACGACa | 105 |
| HvMyb80-11 | TTCAACCCGGCGCAGGCGACGGAGTTCc | 106 |
| HvMyb80-12 | TTCCGGTACGAAGGGACGGCGTCGGGGt | 107 |
| HvMyb80-13 | TTCGTACCGGAACTCCGTCGCCTGCGCc | 108 |
| HvMyb80-14 | TTCCTGCAATGCCGGCCTGCCCCCCTCg | 109 |
| HvMyb80-15 | TTTCCTGCAATGCCGGCCTGCCCCCCTc | 110 |
| HvMyb80-16 | TTCCGCCTCATCGTCGCCGCGGCTGCTg | 111 |
| HvMyb80-17 | TTCCCTCCATCCTTGCCGTCTTCCGCCt | 112 |
| HvMyb80-18 | TTCCCTTCCCTCCATCCTTGCCGTCTTC | 113 |
| HvMyb80-19 | TTCGTGAGCTCGTCGGGCAAGTCCCAGa | 114 |

Plants containing the mutations resulting in the generation of early stop codons TGA, TAA, or TAG) in the ORF are identified based on appropriate molecular technologies (PCR and sequencing). Seeds from the TO-plants are produced. The T1 plants are checked again for the presence of the mutations. Plants containing homozygous mutations in the Myb80 or the Dwarf11 gene are identified. The phenotype of the homozygous plants is determined:

Myb80 homozygous mutant plants show a male sterile phenotype but are not affected in the female fertility and are thus female fertile.

Dwarf11 homozygous mutant plants show a reduced plant height of 10% compared to wt plants and the seeds produced on these plants have a different shape (>10% reduced length) and a reduced weight (the thousand kernel weight is reduced by 10%) compared to either the heterozygous plants or the wt-plants. This difference in the seed shape and the seed weight allows the separation of the seeds harbouring a homozygous mutation of the Dwarf11 gene in a seed processing machine.

In co-transformation experiments using both constructs together, TO-plants are identified showing mutations in the first exon of both genes together.

Plants showing mutations resulting in early stop codons in both genes are identified and T1-seeds are produced form these plants.

T1 plants are checked via appropriate molecular methods for the presence of the expected mutations. Plants homozygous for both mutations are identified and the phenotype of these plants is determined. The plants are smaller than wt plants and are not producing pollen.

Seeds from heterozygous plants produced via selfing are separated based on their seed size. The small sized seeds and the large seeds will be grown separately: The small sized seeds result in dwarf, males sterile plants whereas the larger seeds give normal plants, fully fertile.

### Test of Hybridization

Small sized seeds (male sterile, dwarf) are grown in the field in one row. There is a neighbouring row at each side of the male sterile row in close distance with any barley variety having a similar flowering time. Seeds from each row are harvested separately. The seeds from the middle row are 100% hybrid seeds whereas the seeds from the pollinator lanes are 100% self-pollinated seeds

### Test of Hybrid Vigor

Hybrid seeds deriving from the Test Hybridization is grown in a field trial to compare the yield between the hybrid lines and the parent lines. The yield from the hybrid lines is at least 110% of the yield of the parental lines.

### Screening of a TILLING Population

Mutants based on EMS TILLING are generated and lines with a double mutation are identified. The TILLING experiment is conducted in accordance with the following procedures. Dry seeds are initially soaked in tap water for 10 h until germinated and embryo buds begin to emerge out of the seed coat. The germinated seeds are then incubated in 0.5, 1.0, or 1.5% EMS in 0.1 M sodium phosphate buffer (pH 7.0) for 4 h. Around 2000 seeds are used in each treatment. EMS treatment is performed on a shaker at 50 rpm in dark conditions at 20 C with one seed per milliliter EMS buffer. After incubation, seeds are washed in tap water for 4 h at room temperature and prepared for sowing. (Guo et al., 2017). Repeat all the experiments done with the GE-derived plants also with the TILLING derived plants.

1. A method for generating or modifying a plant or plant part, comprising
a) providing a plant or plant part not expressing or having reduced expression, activity, and/or stability of the Myb80 gene product; and/or having a mutation in the Myb80 gene;
b) reducing or eliminating expression, activity, and/or stability of the Dwarf11 gene product in said plant or plant part; and/or introducing a mutation in the Dwarf11 gene or regulatory sequence thereof.

2. The method according to claim 1, wherein said plant or plant part comprises one or more mutation in the Myb80 gene, preferably wherein said mutation is selected from a nonsense or missense mutation, a frameshift mutation, an indel mutation, a dominant negative mutation, a knock-out mutation, or knock-down mutation.

3. A method for generating or modifying a plant or plant part, comprising
a) providing a plant or plant part not expressing or having reduced expression, activity, and/or stability of the Dwarf11 gene product; and/or having a mutation in the Dwarf11 gene;
b) reducing or eliminating expression, activity, and/or stability of the Myb80 gene product in said plant or plant part; and/or introducing a mutation in the Myb80 gene or regulatory sequence thereof.

4. The method according to claim 3, wherein said plant or plant part comprises one or more mutation in the Dwarf11 gene, preferably wherein said mutation is selected from a nonsense or missense mutation, a frameshift mutation, an indel mutation, a dominant negative mutation, a knock-out mutation, or knock-down mutation.

5. The method according to any of claim 1, wherein said MYB80 gene and said DWARF11 gene are located on the same chromosome, preferably wherein the physical distance between said Myb80 gene and said Dwarf11 gene in the genome of said plant or plant part is at most 1 Mbp.

6. A method for identifying or selecting a plant or plant part, comprising
a) providing a mixture of plants or plant parts resulting from a cross between a first plant and a second plant; wherein said first plant and/or said second plant have a heterozygous or homozygous mutation in the Myb80 gene and the Dwarf11 gene on the same chromosome;
b) selecting a plant or plant part comprising a homozygous mutation in the Dwarf11 gene.

7. The method according to claim 6, wherein selecting a plant or plant part comprising a homozygous mutation in the Dwarf11 gene comprises phenotypic selection.

8. The method according to claim 7, wherein said phenotypic selection comprises selection based on plant height, plant grain size, grain shape, or grain weight.

9. The method according to claim 1, wherein said method is a method for generating or modifying, or for selecting a plant or plant part which is male sterile.

10. A plant or plant part generated, modified, identified, or selected according to the method of claim 1, or offspring thereof.

11. The plant or plant part according to claim 10, comprising a mutation in the Myb80 gene and the Dwarf11 gene.

12. A method for generating hybrid plants or plant parts comprising crossing a first plant having a homozygous mutation in Myb80 and in Dwarf11 with a second plant, and harvesting seeds, and optionally sowing said seeds.

13. A method for developing an assay to phenotypically detect a gene or an allele of a gene of interest in a plant or plant part, comprising screening for the presence of genes located at most 1 Mb up- or downstream in the chromosome comprising said gene of interest, and selecting a gene causing or capable of causing a phenotype or an allele-dependent phenotype in a plant or plant part as a proxy for phenotypically detecting said gene of interest.

14. The method or plant according to claim 1, wherein said plant or plant part is from the family of Poaceae.

15. A polynucleotide having, comprising, consisting of, consisting essentially of, or comprised in or encoding a sequence which is at least 80%, at least 85%, at least 90%, at least 95%, at least 96%, at least 97%, at least 98%, or at least 99% identical to a sequence as set forth in any of SEQ ID NOs: 1-392, a unique fragment thereof, or the complement or reverse complement thereof.
